(19) United States
(12) Patent Application Publication
(10) Pub. No.: US 2010/0280010 A1
(43) Pub. Date: Nov. 4, 2010

(54) CHEMICAL COMPOUNDS

(76) Inventors: Kristjan Gudmundsson, Durham, NC (US); Sharon Davis Boggs, Durham, NC (US); John Franklin Miller, Durman, NC (US); Angilique Christina Svolto, Durham, NC (US)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(21) Appl. No.: 12/161,799

(22) PCT Filed: Jan. 24, 2007
(86) PCT No.: PCT/US07/60939

§ 371 Date: Jul. 23, 2008

Related U.S. Application Data
(60) Provisional application No. 60/761,892, filed on Jan. 25, 2006.

(51) Int. Cl. A61K 31/551; C07D 401/04; C07D 471/04; C07D 243/08; A61K 31/497; A61K 31/437; A61P 31/18

(52) U.S. Cl. 514/218; 544/363; 546/121; 540/575; 514/253.06; 514/300

(57) ABSTRACT

The present invention provides compounds that demonstrate protective effects on target cells from HIV infection in a manner as to bind to chemokine receptor, and which affect the binding of the natural ligand or chemokine to a receptor such as CXCR4 of a target cell.

### FIELD OF THE INVENTION

The present invention provides compounds that demonstrate protective effects on target cells from HIV infection in a manner as to bind to a chemokine receptor, and which affect the binding of the natural ligand or chemokine to a receptor such as CXCR4 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gp120 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with a selective CXCR4 antagonist, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8⁺ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection asthma and allergic airway inflammation, Alzheimer's disease, and arthritis.

Additionally, CXCR4 antagonists may have a role in remodeling and repair of cardiac tissue and preserving cardiac function post myocardial infarction. After myocardial infarction, peripheral and bone marrow derived endothethial progenitor cells are found within the myocardium. It is thought that these cells result in improved ventricular function. This may be due to the production of cytokines that restore function and vascularization or to differentiation of the cells into functional myocardium. CXCL12 and CXCR4 are required for the homing of these stem cells to the myorcardium. In a preclinical study, a CXCR4 antagonist preserved chronic left ventricular function in rats after induction of a myocardial infarction by promoting mobilization and incorporation of bone marrow-derived enothethial progenitor cells into sites of myocardial neovascularization.

The present invention is directed to compounds that can act as agents that modulate chemokine receptor activity. Such chemokine receptors may include, but are not limited to, CCR1, CCR2, CCR3, CCR4, CCR5, CCR6, CCR7, CCR8, CXCR1, CXCR2, CXCR3, CXCR4, and CXCR5.

The present invention provides compounds that demonstrate protective effects on target cells from HIV infection in a manner as to bind to a chemokine receptor, and which affect the binding of the natural ligand or chemokine to a receptor, such as CXCR4 of a target cell.

### SUMMARY OF THE INVENTION

The present invention includes compounds of formula (I):

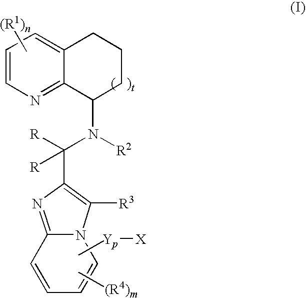

wherein:
t is 0, 1, or 2;
each R independently is H, C₁-C₈ alkyl, C₂-C₆ alkenyl, C₂-C₆ alkynyl, C₁-C₈ haloalkyl, C₃-C₈ cycloalkyl, —R^{a}Ay, —R^{a}OR¹⁰, —R^{a}N(R¹⁰)₂ or —R^{a}S(O)_{q}R¹⁰;
each R¹ independently is halogen, C₁₋₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —N(H)Ay, -Het, —N(H)Het, —OR¹⁰, —OAy, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, —S(O)_{q}Ay, cyano, nitro, or azido;
n is 0, 1, or 2;
m is 0, 1, or 2;
R² is H, C₁-C₈ alkyl, C₂-C₆ alkenyl, C₂-C₆ alkynyl, C₁-C₈ haloalkyl, C₃-C₈ cycloalkyl, —R^{a}cycloalkyl, —R^{a}Ay, —R^{a}OR⁵, or —R^{a}S(O)_{q}R⁵, wherein R² is not amine or alkylamine, or substituted with amine or alkylamine;
R³ is halogen, —N(R¹¹)R¹², —OR¹¹, —C(O)R¹¹, —C(O)N(R¹¹)R¹², —N(R¹¹)C(O)R¹², —N(R¹¹)C(O)N(R¹¹)R¹², —N(R¹¹)C(O)OR¹², —S(O)₂N(R¹¹)R¹², —N(R¹¹)S(O)₂R¹², cyano, nitro, or azido;
each R⁴ independently is halogen, C₁-C₈ haloalkyl, C₁-C₈ alkyl, C₂-C₆ alkenyl, C₂-C₆ alkynyl, C₃-C₈ cycloalkyl, C₃-C₈cycloalkenyl, -Ay, —N(H)Ay, -Het, —N(H)Het, —OR¹⁰, —OAy, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, —S(O)_{q}Ay, —S(O)_{q}Het, cyano, nitro, or azido;
each R⁵ independently is H, C₁-C₈ alkyl, C₂-C₆ alkenyl, C₂-C₆ alkynyl, C₃-C₈ cycloalkyl, or -Ay;
p is 0 or 1;
Y is —NR¹⁰—, —O—, —C(O)NR¹⁰—, —N(R¹⁰)C(O)—, —C(O)—, —C(O)O—, —N(R¹⁰)C(O)N(R¹⁰)—, —S(O)_{q}—, —S(O)_{q}N(R¹⁰)—, or —N(R¹⁰)S(O)_{q}—;
X is —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₈ cycloalkylene, C₂-C₆ alkenylene, C₃-C₈ cycloalkenylene, or C₂-C₆ alkynylene, and is optionally substituted with one or more C₁-C₈ alkyl, hydroxyl or oxo;
each R¹⁰ independently is H, C₁-C₈ alkyl, C₃-C₈ cycloalkyl, C₂-C₆ alkenyl, C₂-C₆ alkynyl, C₃-C₈ cycloalkenyl, —R^{a}cycloalkyl, —R^{a}OR⁵, R^{a}N(R⁶)R⁷ or R^{a}Het.
each R¹¹ and R¹² independently is H, C₁-C₈ alkyl, C₃-C₈ cycloalkyl, C₂-C₈ alkenyl, C₂-C₆ alkynyl, C₃-C₈ cycloalkenyl, —R^{a}cycloalkyl, —R^{a}OR⁵,
each of R⁶ and R⁷ independently are selected from H, C₁-C₈ alkyl, C₁-C₈ alkenyl, C₂-C₆ alkynyl, C₃-C₈ cycloalkyl, C₃-C₈ cycloalkenyl, —R^{a}cycloalkyl, —R^{a}OR⁵, —R^{a}N(R⁸)R⁹, -Ay, -Het, —R^{a}Ay, —R^{a}Het, —C(O)R⁵ or —S(O)_{q}R⁵;
each of R⁸ and R⁹ independently are selected from H or C₁-C₈ alkyl;
each q independently is 0, 1, or 2;
each Ay independently represents a C₃-C₁₀ aryl group optionally substituted with one or more of C₁-C₈ alkyl, C₂-C₆ alkenyl, C₂-C₆ alkynyl, C₁-C₃ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; and
each Het independently represents a C₃-C₇ heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; or pharmaceutically acceptable salts or solvates thereof.

The present invention features a compound of formula (I) wherein, t is 1 or 2 and all other substituents are as defined above or a pharmaceutically acceptable derivative thereof. The present invention features a compound of formula (I) wherein t is 1 and all other substituents are as defined above or a pharmaceutically acceptable derivative thereof.

The present invention features a compound of formula (I) wherein each R is H or alkyl and all other substituents are as defined above or a pharmaceutically acceptable derivative thereof. The present invention also features a compound of formula (I) wherein each R is H.

The present invention features a compound of formula (I) wherein n is 0 and all other substituents are as defined above or a pharmaceutically acceptable derivative thereof.

The present invention features a compound of formula (I) wherein n is 1 and R¹ is halogen, C₁-C₈ haloalkyl, C₁-C₈ alkyl, OR¹⁰, N(R⁶)R⁷, CO₂R¹⁰, C(O)N(R⁶)R⁷, or cyano and all other substituents are as defined above or a pharmaceutically acceptable derivative thereof.

The present invention features a compound of formula (I) wherein R² is H, C₁-C₈alkyl, C₁-C₈haloalkyl, —R^{a}cycloalkyl, R^{a}OR⁵, or C₃-C₈cycloalkyl and all other substituents are as defined above or a pharmaceutically acceptable derivative thereof. The present invention features a compound of formula (I) wherein R² is C₁-C₈alkyl, R^{a}OR⁵ or C₃-C₈cycloalkyl. The present invention features a compound of formula (I) wherein R² is C₁-C₅alkyl.

The present invention features a compound of formula (I) wherein R³ is halogen, —C(O)R¹¹, —C(O)N(R¹¹)R¹², —S(O)₂N(R¹¹)R¹², —N(R¹¹)C(O)N(R¹¹)R¹² or cyano and all other substituents are as defined above or a pharmaceutically acceptable derivative thereof. The present invention features a compound of formula (I) wherein R³ is —C(O)N(R¹¹)R¹² or —S(O)₂N(R¹¹)R¹² and all other substituents are as defined above or a pharmaceutically acceptable derivative thereof. The present invention features a compound of formula (I) wherein R³ is halogen and all other substituents are as defined above or a pharmaceutically acceptable derivative thereof.

The present invention features a compound of formula (I) wherein m is 0 and all other substituents are as defined above or a pharmaceutically acceptable derivative thereof.

The present invention features a compound of formula (I) wherein m is 1 or 2 and R⁴ is one or more of halogen, C₁-C₈haloalkyl, C₁-C₈alkyl, OR¹⁰, N(R⁶)R⁷, CO₂R¹⁰, C(O)N(R⁶)R⁷, or cyano and all other substituents are as defined above or a pharmaceutically acceptable derivative thereof. The present invention features a compound of formula (I) wherein m is 1 and all other substituents are as defined above or a pharmaceutically acceptable derivative thereof. The present invention features a compound of formula (I) wherein m is 1 and R⁴ is C₁-C₈ alkyl or halogen.

The present invention features a compound of formula (I) wherein p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof. The present invention features a compound of formula (I) wherein X is —R^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof. The present invention features a compound of formula (I) wherein X is R^{a}N(R¹⁰)₂, -Het, —R^{a}Het, or -HetN(R¹⁰)₂ and all other substituents are as defined above or a pharmaceutically acceptable derivative thereof.

The present invention features a compound of formula (I) wherein p is 1; Y is —N(R¹⁰)—, C(O)N(R¹⁰)—, —N(R¹⁰)C(O)—, or —S(O)_{q}N(R¹⁰)—; and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof. The present invention features a compound of formula (I) wherein Y is —N(R¹⁰)—, —O—, —C(O)N(R¹⁰)—, —N(R¹⁰)C(O)— and X is —R^{a}N(R¹⁰)₂, -Het, —R^{a}Het, or -HetN(R¹⁰)₂ and all other substituents are as defined above or a pharmaceutically acceptable derivative thereof,

The present invention features a compound of formula (I) wherein Het is piperidine, piperazine, azetidine, pyrrolidine, imidazole, pyridine, and the like and all other substituents are as defined above or a pharmaceutically acceptable derivative thereof.

The present invention features a compound of formula (I) wherein, each R is H and t is 1 and all other substituents are as defined above or a pharmaceutically acceptable derivative thereof.

The present invention features a compound of formula (I) wherein p is 0 and X is -Het and all other substituents are as defined above or a pharmaceutically acceptable derivative thereof.

The present invention features a compound of formula (I) wherein p is 0 and X is -HetN(R¹⁰)₂, R¹⁰ is H or C₁-C₈alkyl and all other substituents are as defined above or a pharmaceutically acceptable derivative thereof.

The present invention features a compound of formula (I) wherein -Het is unsubstituted or substituted with one or more C₁-C₈ alkyl or C₃-C₈ cycloalkyl and all other substituents are as defined above or a pharmaceutically acceptable derivative thereof. The present invention features a compound of formula (I) wherein -Het is piperazine, piperidine, C₁-C₈ alkyl substituted piperazine, or C₁-C₈ alkyl substituted piperidine and all other substituents are as defined above or a pharmaceutically acceptable derivative thereof.

The present invention features a compound of formula (I) wherein, the substituent —Yₚ—X is located on the depicted imidazopyridine ring as in formula (I-A):

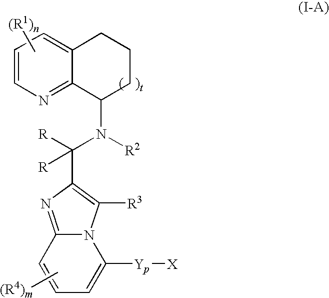

wherein all substituents are as defined above, or pharmaceutically acceptable derivatives thereof.

One aspect of the invention includes compounds of formula (I-A) wherein p is 0 and X is -Het and all other substituents are as defined above or a pharmaceutically acceptable derivative thereof.

One aspect of the invention includes compounds of formula (I-A) wherein p is 0 and X is -HetN(R¹⁰)₂ and R¹⁰ is H or C₁-C₈alkyl and all other substituents are as defined above or a pharmaceutically acceptable derivative thereof.

One aspect of the invention includes compounds of formula (I-A) where t is 1 or 2; R is H or C₁-C₈ alkyl; R² is H, C₁-C₈ alkyl, C₃-C₈ cycloalkyl, or —R^{a}cycloalkyl; R³ is halogen, —C(O)R¹¹, —C(O)N(R¹¹)R¹², —S(O)₂N(R¹¹)R¹² or cyano; n is 0; m is 0 or 1; p is 0 and X is -Het or -HetN(R¹⁰)₂, R¹⁰ is H or C₁-C₈ alkyl and -Het is unsubstituted or substituted with C₁-C₈ alkyl or C₃-C₈ cycloalkyl.

One aspect of the invention includes compounds of formula (I-A) where t is 1 or 2; R is H or C₁-C₈ alkyl; R² is H, C₁-C₈alkyl, C₃-C₈ cycloalkyl, or —R^{a}cycloalkyl; R³ is halogen, —C(O)R¹¹, —C(O)N(R¹¹)R¹², —S(O)₂N(R¹¹)R¹² or cyano; n is 0; m is 0 or 1; p is 1; Y is —N(R¹⁰)—, —O—, —C(O)N(R¹⁰)—, or —N(R¹⁰)— or —N(R¹⁰)C(O)—; X is -Het or -HetN(R¹⁰)₂, and R¹⁰ is H or C₁-C₈ alkyl and Het is unsubstituted or substituted with C₁-C₈ alkyl or C₃-C₈ cycloalkyl.

One aspect of the invention includes compounds of formula (I-A) where t is 1 or 2; R is H or C₁-C₈ alkyl; R² is H, C₁-C₈ alkyl, C₃-C₈ cycloalkyl, or —R^{a}cycloalkyl; R³ is halogen, —C(O)R¹¹, —C(O)N(R¹¹)R¹², —S(O)₂N(R¹¹)R¹² or cyano; n is 0; m is 0 or 1; p is 1; Y is —N(R¹⁰)— or —O— and X is -Het, unsubstituted or substituted with C₁-C₈ alkyl or C₃-C₈ cycloalkyl.

Compounds of the present invention include:
5-(4-Methyl-1-piperazinyl)-2-({methyl[(8S)-5,6,7,8-tetrahydro-8-quinolinyl]amino}methyl)imidazo[1,2-a]pyridine-3-carbonitrile;
5-(4-Methyl-1-piperazinyl)-2-({methyl[(8S)-5,6,7,8-tetrahydro-8-quinolinyl]amino}methyl)imidazo[1,2-a]pyridine-3-carboxamide;
2-({Ethyl[(8S)-5,6,7,8-tetrahydro-8-quinolinyl]amino}methyl)-5-(4-methyl-1-piperazinyl)imidazo[1,2-a]pyridine-3-carbonitrile;
2-({Ethyl[(8S)-5,6,7,8-tetrahydro-8-quinolinyl]amino}methyl)-5-(4-methyl-1-piperazinyl)imidazo[1,2-a]pyridine-3-carboxamide;
5-(4-Methyl-1-piperazinyl)-2-({propyl[(8S)-5,6,7,8-tetrahydro-8-quinolinyl]amino}methyl)imidazo[1,2-a]pyridine-3-carbonitrile;
5-(4-Methyl-1-piperazinyl)-2-({propyl[(8S)-5,6,7,8-tetrahydro-8-quinolinyl]amino}methyl)imidazo[1,2-a]pyridine-3-carboxamide;
2-({(1-Methylethyl)[(8S)-5,6,7,8-tetrahydro-8-quinolinyl]amino}methyl)-5-(4-methyl-1-piperazinyl)imidazo[1,2-a]pyridine-3-carbonitrile;
2-({(1-Methylethyl)[(8S)-5,6,7,8-tetrahydro-8-quinolinyl]amino}methyl)-5-(4-methyl-1-piperazinyl)imidazo[1,2-a]pyridine-3-carboxamide;
2-({(Cyclopropylmethyl)[(8S)-5,6,7,8-tetrahydro-8-quinolinyl]amino}methyl)-5-(4-methyl-1-piperazinyl)imidazo[1,2-a]pyridine-3-carbonitrile;
5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({(phenylmethyl)[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({methyl[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({methyl[(8S)-5,6,7,8-tetrahydro-8-quinolinyl]amino}methyl)imidazo[1,2-a]pyridine-3-carboxamide;
5-[4-(dimethylamino)-1-piperidinyl]-2-({ethyl[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({ethyl[(8S)-5,6,7,8-tetrahydro-8-quinolinyl]amino}methyl)imidazo[1,2-a]pyridine-3-carboxamide;
5-[4-(dimethylamino)-1-piperidinyl]-2-({propyl[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({propyl[(8S)-5,6,7,8-tetrahydro-8-quinolinyl]amino}methyl)imidazo[1,2-a]pyridine-3-carboxamide;
5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carboxamide;
(8S)—N-{[3-Chloro-5-(4-methyl-1-piperazinyl)imidazo[1,2-a]pyridin-2-yl]methyl}-N-methyl-5,6,7,8-tetrahydro-8-quinolinamine;
(8S)—N-{[3-Chloro-5-(4-ethyl-1-piperazinyl)imidazo[1,2-a]pyridin-2-yl]methyl}-N-methyl-5,6,7,8-tetrahydro-8-quinolinamine;
(8S)—N-({3-Chloro-5-[(3R)-3-(dimethylamino)-1-pyrrolidinyl]imidazo[1,2-a]pyridin-2-yl}methyl)-N-methyl-5,6,7,8-tetrahydro-8-quinolinamine;
(8S)—N-({3-Chloro-5-[4-(dimethylamino)-1-piperidinyl]imidazo[1,2-a]pyridin-2-yl}methyl)-N-methyl-5,6,7,8-tetrahydro-8-quinolinamine;
(8S)—N-{[3-Chloro-5-(4-methylhexahydro-1H-1,4-diazepin-1-yl)imidazo[1,2-a]pyridin-2-yl]methyl}-N-methyl-5,6,7,8-tetrahydro-8-quinolinamine;
N-[3-Chloro-2-({methyl[(8S)-5,6,7,8-tetrahydro-8-quinolinyl]amino}methyl)imidazo[1,2-a]pyridin-5-yl]-N,N′,N′-trimethyl-1,2-ethanediamine;
(8S)—N-({3-Chloro-5-[(3S)-3-methyl-1-piperazinyl]imidazo[1,2-a]pyridin-2-yl}methyl)-N-methyl-5,6,7,8-tetrahydro-8-quinolinamine;
(8S)—N-({3-Chloro-5-[(3S)-3,4-dimethyl-1-piperazinyl]imidazo[1,2-a]pyridin-2-yl}methyl)-N-methyl-5,6,7,8-tetrahydro-8-quinolinamine;
(8S)—N-({3-Chloro-5-[4-(1-methylethyl)-1-piperazinyl]imidazo[1,2-a]pyridin-2-yl}methyl)-N-methyl-5,6,7,8-tetrahydro-8-quinolinamine;
(8S)—N-({3-Chloro-5-[(8aS)-hexahydropyrrolo[1,2-a]pyrazin-2(1H)-yl]imidazo[1,2-a]pyridin-2-yl}methyl)-N-methyl-5,6,7,8-tetrahydro-8-quinolinamine;
1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanone;
(8S)—N-{[3-Bromo-5-(4-methyl-1-piperazinyl)imidazo[1,2-a]pyridin-2-yl]methyl}-N-methyl-5,6,7,8-tetrahydro-8-quinolinamine;
(8S)—N-({5-[4-(dimethylamino)-1-piperidinyl]imidazo[1,2-a]pyridin-2-yl}methyl)-N-propyl-5,6,7,8-tetrahydro-8-quinolinamine;
(8S)—N-{[8-Fluoro-5-(4-methyl-1-piperazinyl)imidazo[1,2-a]pyridin-2-yl]methyl}-N-propyl-5,6,7,8-tetrahydro-8-quinolinamine; and pharmaceutically acceptable derivatives thereof.

One aspect of the present invention includes the compounds substantially as hereinbefore defined with reference to any one of the Examples.

One aspect of the present invention includes a pharmaceutical composition comprising one or more compounds of the present invention and a pharmaceutically acceptable carrier.

One aspect of the present invention includes one or more compounds of the present invention for use as an active therapeutic substance.

One aspect of the present invention includes one or more compounds of the present invention for use in the treatment (including prophylaxis) of diseases and conditions caused by inappropriate activity of CXCR4.

One aspect of the present invention includes one or more compounds of the present invention for use in the treatment (including prophylaxis) of HIV infection, myocardial infarction, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. In one embodiment the condition or disease is HIV infection, rheumatoid arthritis, inflammation, or cancer. In yet another embodiment the disease is HIV infection.

One aspect of the present invention includes the use of one or more compounds of the present invention in the manufacture of a medicament for use in the treatment (including prophylaxis) of a condition or disease modulated by a chemokine receptor. Preferably the chemokine receptor is CXCR4.

One aspect of the present invention includes use of one or more compounds of the present invention in the manufacture of a medicament for use in the treatment (including prophylaxis) of HIV infection, myocardial infarction, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the use relates to a medicament wherein the condition or disorder is HIV infection, rheumatoid arthritis, inflammation, or cancer.

One aspect of the present invention includes a method for the treatment (including prophylaxis) of a condition or disease modulated by a chemokine receptor comprising the administration of one or more compounds of the present invention. Preferably the chemokine receptor is CXCR4.

One aspect of the present invention includes a method for the treatment (including prophylaxis) of HIV infection, myocardial infarction, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of one or more compounds of the present invention.

One aspect of the present invention includes a method for the treatment (including prophylaxis) of HIV infection, rheumatoid arthritis, inflammation, or cancer comprising the administration of one or more compounds of the present invention. One aspect of the invention includes a method for the treatment (including prophylaxis) of HIV infection.

### DETAILED DESCRIPTION OF THE INVENTION

Terms are used within their accepted meanings. The following definitions are meant to clarify, but not limit, the terms defined.

As used herein the term “alkyl” alone or in combination with any other term, refers to a straight or branched chain hydrocarbon, containing from one to twelve carbon atoms, unless specified otherwise. Examples of “alkyl” as used herein include, but are not limited to, methyl, ethyl, propyl, isopropyl, isobutyl, n-butyl, tert-butyl, sec-butyl, isopentyl, n-pentyl, n-hexyl, and the like.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds, which may occur at any stable point along the chain. Examples include, but are not limited to, ethynyl, propynyl, butynyl, pentynyl, and the like.

As used herein, the term “alkylene” refers to an optionally substituted straight or branched chain divalent hydrocarbon radical, preferably having from one to ten carbon atoms, unless specified otherwise. Examples of “alkylene” as used herein include, but are not limited to, methylene, ethylene, n-propylene, n-butylene, and the like. Preferred substituent groups include C₁-C₈ alkyl, hydroxyl or oxo.

As used herein, the term “alkenylene” refers to a straight or branched chain divalent hydrocarbon radical, preferably having from two to ten carbon atoms, unless specified otherwise, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from two to ten carbon atoms, unless otherwise specified, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Unless otherwise indicated, cycloalkyl is composed of three to eight carbon atoms, Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “heterocycle”, “heterocyclic” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. More preferably, the heteroatom is N.

Preferably the heterocyclyl ring is three to twelve-membered, unless otherwise indicated, and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, tetrahydrothiopyran, aziridine, azetidine and tetrahydrothiophene. When the heterocyclic ring has substituents, it is understood that the substituents may be attached to any atom in the ring, whether a heteroatom or a carbon atom, provided that a stable chemical structure results. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “aryl” refers to an optionally substituted carbocyclic aromatic moiety (such as phenyl or naphthyl) containing the specified number of carbon atoms, preferably 6-14 carbon atoms or 6-10 carbon atoms. The term aryl also refers to optionally substituted ring systems, for example anthracene, phenanthrene, or naphthalene ring systems. Examples of “aryl” groups include, but are not limited to, phenyl, naphthyl, indenyl, azulenyl, fluorenyl, anthracenyl, phenanthrenyl, tetrahydronaphthyl, indanyl, phenathridinyl, and the like. Unless otherwise indicated, the term aryl also includes each possible positional isomer of an aromatic hydrocarbon radical, such as 1-naphthyl, 2-naphthyl, 5-tetrahydronaphthyl, 6-tetrahydronaphthyl, 1 phenanthridinyl, 2-phenanthridinyl, 3-phenanthridinyl, 4-phenanthridinyl, and the like. Preferred substituent groups include C₁-C₈ alkyl, C₂-C₆ alkenyl, C₂-C₈ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “heteroaryl” refers to an optionally substituted monocyclic five to seven membered aromatic ring unless otherwise specified, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Preferably, the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, and pyrazolopyrimidinyl. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and alkylamino.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to a group —OR′, where R′ is alkyl as defined. Examples of suitable alkoxy radicals include, but are not limited to, methoxy, ethoxy, propoxy, isopropoxy, n-butoxy, isobutoxy, sec-butoxy, tert-butoxy, and the like.

As used herein the term “cycloalkoxy” refers to a group —OR′, where R′ is cycloalkyl as defined.

As used herein the term “alkoxycarbonyl” refers to groups such as:

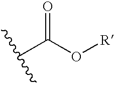

where the R′ represents an alkyl group as herein defined.

As used herein the term “aryloxycarbonyl” refers to groups such as:

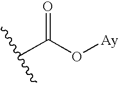

where the Ay represents an aryl group as herein defined.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached.

As used herein the term “amide” refers to a group —C(O)NR′R″, where R′ and R″ independently represent H, alkyl, alkenyl, alkynyl, cycloalkyl, heterocyclyl, aryl, or heteroaryl.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for modifications, which are encompassed within the scope of the appended claims.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point. Though a crystalline form of compounds of the present invention are generally preferred, the invention also contemplates amorphous forms of the compounds produced by methods known in the art (e.g. spray drying, milling, freeze drying, and so forth).

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds of the present invention, as well as any wholly or partially equilibrated mixtures thereof. The present invention also includes the individual isomers of the compounds represented by the formulas above as mixtures with isomers thereof in which one or more chiral centers are inverted.

As used herein, the term “solvate” refers to a complex of variable stoichiometry formed by a solute (in this invention, a compound of the present invention, or a salt or other pharmaceutically acceptable derivative thereof) and a solvent. Such solvents, for the purpose of the invention, should not interfere with the biological activity of the solute. Non-limiting examples of suitable solvents include, but are not limited to water, methanol, ethanol, ethyl acetate, acetone, acetonitrile, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “pharmaceutically acceptable derivative” means any pharmaceutically acceptable salt, ester, salt of an ester, ether, amides, or other derivative of a compound of this invention which, upon administration to a recipient, is capable of providing directly or indirectly a compound of this invention or an inhibitorily active metabolite or residue thereof. Particularly favored derivatives and prodrugs are those that increase the bioavailability of the compounds of this invention when such compounds are administered to a mammal, for example, by allowing an orally administered compound to be more readily absorbed into the blood, or which enhance delivery of the parent compound to a biological compartment, for example, the brain or lymphatic system, relative to the parent species.

Salts of the compounds of the present invention may be made by methods known to a person skilled in the art. For example, treatment of a compound of the present invention with an appropriate base or acid in an appropriate solvent will yield the corresponding salt.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

Pharmaceutically acceptable salts of the compounds according to the invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts.

Other compounds of this invention may be prepared by one skilled in the art following the teachings of the specification coupled with knowledge in the art using reagents that are readily synthesized or commercially available.

Any reference to any of the above compounds also includes a reference to a pharmaceutically acceptable salt thereof.

Esters of the compounds of the present invention are independently selected from the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

In such esters, unless otherwise specified, any alkyl moiety present advantageously contains from 1 to 18 carbon atoms, particularly from 1 to 6 carbon atoms, more particularly from 1 to 4 carbon atoms, Any cycloalkyl moiety present in such esters advantageously contains from 3 to 6 carbon atoms. Any aryl moiety present in such esters advantageously comprises a phenyl group.

Ethers of the compounds of the present invention include, but are not limited to methyl, ethyl, butyl and the like.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators.

In one aspect of the present invention, the compounds demonstrate protective effects against HIV infection by inhibiting binding of HIV to a chemokine receptor such as CXCR4 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis), systemic anaphylaxis or hypersensitivity responses, drug allergies, insect sting allergies, autoimmune diseases such as rheumatoid arthritis, psoriatic arthritis, systemic lupus erythematosus, myastenia gravis, juvenile onset diabetes, glomerulonephritis, autoimmune throiditis, graft rejection, including allograft rejection or graft-versus-host disease, inflammatory bowel diseases, such as Crohn's disease and ulcerative colitus, spondyloarthropathies, scleroderma, psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis), eoosinophilic myotis, eosinophilic fasciitis, and cancers.

The present invention features compounds according to the invention for use in medical therapy, for example for the treatment (including prophylaxis) of a viral infection, for example an HIV infection and associated conditions. The compounds according to the invention are especially useful for the treatment of AIDS and related clinical conditions such as AIDS related complex (ARC), progressive generalized lymphadenopathy (PGL), Kaposi's sarcoma, thromobocytopenic purpura, AIDS-related neurological conditions such as AIDS dementia complex, multiple sclerosis or tropical paraperesis, anti-HIV antibody-positive and HIV-positive conditions, including such conditions in asymptomatic patients.

The present invention further provides a method for the treatment of a clinical condition in a patient, for example, a mammal including a human which clinical condition includes those which have been discussed hereinbefore, which comprises treating said patient with a pharmaceutically effective amount of a compound according to the invention. The present invention also includes a method for the treatment (including prophylaxis) of any of the aforementioned diseases or conditions.

According to another aspect, the present invention provides a method for the treatment or prevention of the symptoms or effects of a viral infection in an infected patient, for example, a mammal including a human, which comprises administering to said patient a pharmaceutically effective amount of a compound according to the invention. According to one aspect of the invention, the viral infection is a retroviral infection, in particular an HIV infection.

The present invention further includes the use of a compound according to the invention in the manufacture of a medicament for administration to a subject for the treatment of a viral infection, in particular and HIV infection.

The compounds according to the invention may also be used in adjuvant therapy in the treatment of HIV infections or HIV-associated symptoms or effects, for example Kaposi's sarcoma. Reference herein to treatment extends to prophylaxis as well as the treatment of established conditions, disorders and infections, symptoms thereof, and associated clinical conditions. The above compounds according to the invention and their pharmaceutically acceptable derivatives may be employed in combination with other therapeutic agents for the treatment of the above infections or conditions. Combination therapies according to the present invention comprise the administration of a compound of the present invention or a pharmaceutically acceptable derivative thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

For use in therapy, therapeutically effective amounts of a compound of the present invention, as well as salts, solvates, or other pharmaceutically acceptable derivatives thereof, may be administered as the raw chemical. Additionally, the active ingredient may be presented as a pharmaceutical composition.

Accordingly, the invention further provides pharmaceutical compositions that include effective amounts of compounds of the present invention and salts, solvates, or other pharmaceutically acceptable derivatives thereof, and one or more pharmaceutically acceptable carriers, diluents, or excipients. The compounds of the present invention and salts, solvates, or other pharmaceutically acceptable derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the present invention or salts, solvates, or other pharmaceutically acceptable derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of the present invention for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for a 70 kg adult mammal one example of an actual amount per day would usually be from 7 to 700 mg. This amount may be given in a single dose per day or in a number (such as two, three, four, five, or more) of sub-doses per day such that the total daily dose is the same. An effective amount of a salt, solvate, or other pharmaceutically acceptable derivative thereof, may be determined as a proportion of the effective amount of the compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the present invention and salts, solvates, or other pharmaceutically acceptable derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of the present invention and salts, solvates, or other pharmaceutically acceptable derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, or other pharmaceutically acceptable derivatives thereof, may be employed alone or in combination with other therapeutic agents. The compound(s) of the present invention and the other pharmaceutically active agent(s) may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, emtricitabine, alovudine, amdoxovir, elvucitabine, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, TMC-278, TMC-125, etravirine, and similar agents;

Protease inhibitors such as saquinavir, ritonavir, indinavir, nelfinavir, amprenavir, fosamprenavir, brecanavir, atazanavir, tipranavir, palinavir, lasinavir, and similar agents;

Entry inhibitors such as enfuvirtide (T-20), T-1249, PRO-542, PRO-140, TNX-355, BMS-806, 5-Helix and similar agents;

Integrase inhibitors such as L-870,180 and similar agents;

Budding inhibitors such as PA-344 and PA-457, and similar agents; and

Other CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

The compounds of the present invention may be used in the treatment of a variety of disorders and conditions and, as such, the compounds of the present invention may be used in combination with a variety of other suitable therapeutic agents useful in the treatment (including prophylaxis) of those disorders or conditions. The compounds may be used in combination with any other pharmaceutical composition where such combined therapy may be useful to modulate chemokine receptor activity and thereby prevent and treat inflammatory and/or immunoregulatory diseases.

It should be understood that in addition to the ingredients particularly mentioned above, the pharmaceutical compositions of this invention may include other agents conventional in the art having regard to the type of pharmaceutical composition in question, for example, those suitable for oral administration may include such further agents as sweeteners, thickeners, and flavoring agents.

The compounds of the present invention may be prepared according to the following reaction schemes and examples, or modifications thereof using readily available starting materials, reagents and conventional synthesis procedures. In these reactions, it is also possible to make use of variants which are know to those of ordinary skill in the art.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

Those skilled in the art will recognize if a stereocenter exists in compounds of the present invention. Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

### Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
RT (room temperature); h (hours);
min (minutes); TLC (thin layer chromatography);
mp (melting point); RP (reverse phase);
Tᵣ (retention time); TFA (trifluoroacetic acid);
TEA (triethylamine); THF (tetrahydrofuran);
TFAA (trifluoroacetic anhydride); CD₃OD (deuterated methanol);
CDCl₃ (deuterated chloroform); DMSO (dimethylsulfoxide);
SiO₂ (silica); atm (atmosphere);
EtOAc (ethyl acetate); CHCl₃ (chloroform);
HCl (hydrochloric acid); Ac (acetyl);
DMF (N,N-dimethylformamide); Me (methyl);
Cs₂CO₃ (cesium carbonate); EtOH (ethanol);
Et (ethyl); tBu (tert-butyl);
MeOH (methanol) p-TsOH (p-toluenesulfonic acid);
POCl₃ (phosphorus oxychloride)
MP-TsOH (polystyrene resin bound equivalent of p-TsOH from Argonaut Technologies).

Unless otherwise indicated, all temperatures are expressed in ° C. (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds was assigned by Ab Initio Vibrational Circular Dichroism (VCD) Spectroscopy. The experimental VCD spectra were acquired in CDCl₃ using a Bomem Chiral® VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R)- or (S)-configuration. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A.11.4, M. J. Frisch et al., Gaussian, Inc., Pittsburgh Pa., 2002.

Compounds of formula (I) where R is H, t is 1 and all other variables are as defined herein can be prepared according to Scheme 1:

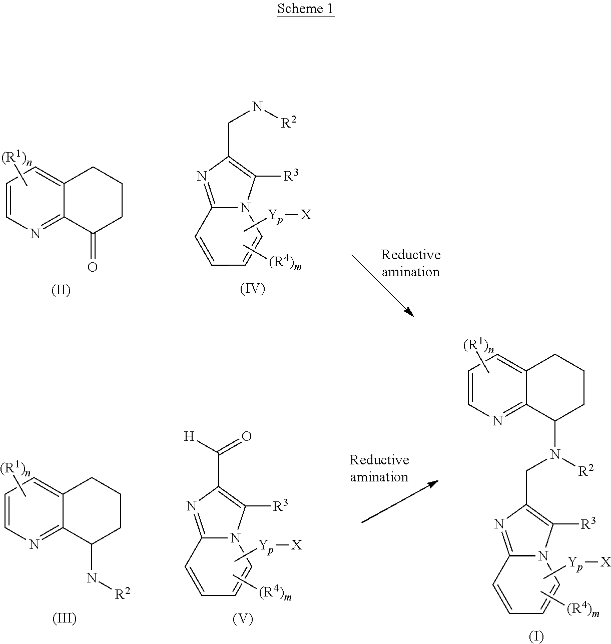

More specifically, compounds of formula (I) can be prepared by reacting a compound of formula (II) with a compound (IV) or alternatively reacting a compound of formula (III) with a compound of formula (V) under reductive conditions. The reductive amination can be carried out by treating the compound of formula (II) or (III) with a compound of formula (IV) or (V) in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

Compounds of formula (II) can be prepared as described in the literature (J. Org. Chem., 2002, 67, 2197-2205, herein incorporated by reference with regard to such synthesis). Compounds of formula (III) can be prepared by reductive amination of compounds of formula (II) using processes well known to those skilled in the art of organic synthesis. Compounds of formula (V) can be prepared by methods similar to those described in the literature (J. Heterocyclic Chemistry, 1992, 29, 691-697, incorporated by reference with regard to such synthesis). Compounds of formula (IV) can be prepared from compounds of formula (V) via reductive amination using processes known to those skilled in the art.

As is evident to one skilled in the art a compound where R is not H can be prepared in a similar fashion as outlined in Scheme 1. Also evident to one skilled in the art is that compounds of formula (I) where t is 0 or t is 2 can be prepared in a similar fashion as outlined in Scheme 1.

Compounds of formula (I) wherein R is H and t is 1 and all other variables are as defined in connection with formula (I) can be prepared according to Scheme 2, where LV is a suitable leaving group (e.g., halogen, mesylate, tosylate, or the like):

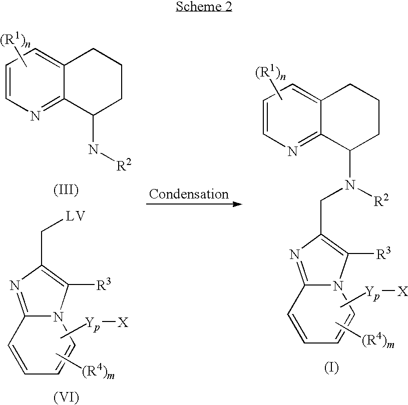

Compounds of formula (I) can be prepared by reacting a compound of formula (III) with a compound of formula (VI) where LV is a leaving group (e.g., halogen, mesylate, tosylate, or the like). This condensation is typically carried out in a suitable solvent optionally in the presence of base, optionally with heating. Suitable solvents include tetrahydrofuran, dioxane, acetonitrile, nitromethane, N,N-dimethylformamide, and the like. Suitable bases include triethylamine, pyridine, dimethylaminopyridine, N,N-diisopropylethylamine, potassium carbonate, sodium carbonate, cesium carbonate and the like. The reaction can be carried out at room temperature or optionally heated to 30-200° C. Optionally the reaction can be carried out in a microwave. A catalyst, such as potassium iodide, tertbutylammonium iodide, or the like, can optionally be added to the reaction mixture. Compounds of formula (VI) can be prepared by methods similar to those described in the literature (Chem. Pharm. Bull. 2000, 48, 935; Tetrahedron, 1991, 47, 5173; Tetrahedron Lett. 1990, 31, 3013; J. Heterocyclic Chemistry, 1988, 25, 129; Chemistry of Heterocyclic Compounds, 2002, 38, 590; each incorporated by reference with regard to such synthesis).

Compounds of formulas (I-B) and (I-C) wherein t is 1, each R is H, and all other variables are as defined above, can be prepared according to Scheme 3. Compounds of formula (I-B) and formula (I-C) wherein t is 0 or 2 and R is other than H can be made in a similar fashion as would be evident to one of skill in the art.

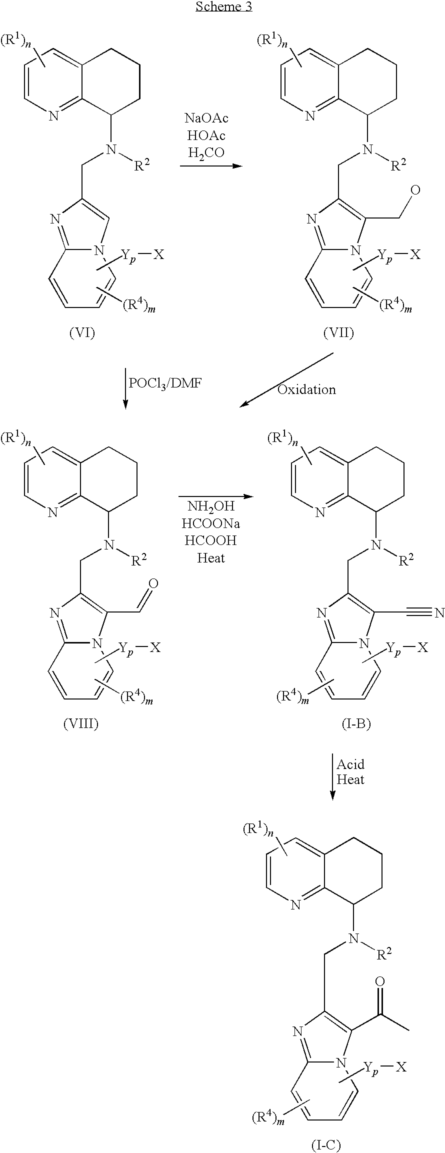

Generally, the process for preparing the compounds of formula (I-B) wherein t is 1, each R is H and all other variables are as defined herein above in connection with formula (I) comprises the steps of:

(a) preparing a compound of formula (VI) from a compound of formula (II) or (III) and a compound similar to a compound of formula (IV) or (V), but where R3 is H, respectively, by reductive amination;
(b) preparing a compound of formula (VIII) from a compound of formula (VI) via either of two methods:
1) formylation of a compound of formula (VI) using Vilsmeier Haack type conditions to give a compound of formula (VIII) or
2) hydroxymethylation of a compound of formula (VI) to give a hydroxymethyl compound of formula (VII) followed by oxidation to give a compound of formula (VIII);
   (c) preparing a compound of formula (I-B) from a compound of formula (VII) by treatment with hydroxylamine hydrochloride and sodium formate in formic acid with heating.

More specifically compounds of formula (VII) can be obtained by treatment of compound of formula (VI) with formaldehyde in the presence of acid, optionally in the presence of a solvent. The solvent can be acetic acid or an inert solvent such as water and the like. Optionally the reaction can be conducted at room temperature or with heating to 100° C. The reaction conditions are related to conditions described in the literature for hydroxymethylation of other imidazopyridines (e.g. Bioorganic and Medicinal Chemistry 2002, 10, 941-946; J. Med. Chem. 1998, 41, 5108-5112 incorporated herein by reference with regard to such synthesis).

A compound of formula (VIII) can be obtained by oxidation of a compound of formula (VII). The oxidation is typically carried out in an inert solvent using a suitable oxidant. Suitable solvents include dichloromethane, chloroform, tetrahydrofuran and the like. Suitable oxidants include Dess Martin periodinane oxidation, preferentially using a Dess Marin periodinane on a solid support. The reaction can be carried out at room temperature or optionally with heating.

Alternatively, a compound of formula (VIII) can be prepared by formylation of compound of formula (VI) using Vilsmeier Haack formylation conditions (e.g. POCl₃ and DMF) or other formylation conditions well know to those skilled in the art of organic chemistry.

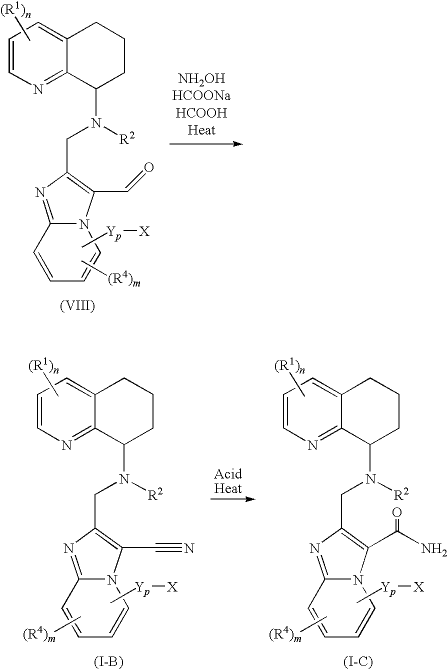

Nitrile compounds of formula (I-B) can be prepared from a compound of formula (VIII) by treatment with hydroxylamine hydrochloride and sodium formate in formic acid with heating.

Amide compounds of formula (I-C) can be prepared by hydrolyzis of a compound of formula (I-B) wherein the variables are as defined with respect to Scheme 3 under suitable conditions. Suitable conditions include treatment of compound of formula (I-C) with strong acids, such as sulfuric acid, optionally in the presence of a suitable solvent and optionally with heating.

Compounds of formula (I-D) where R is H, Hal is halogen and all other variables are as defined in connection with compound of formula (I) can be prepared as outlined in Scheme 4.

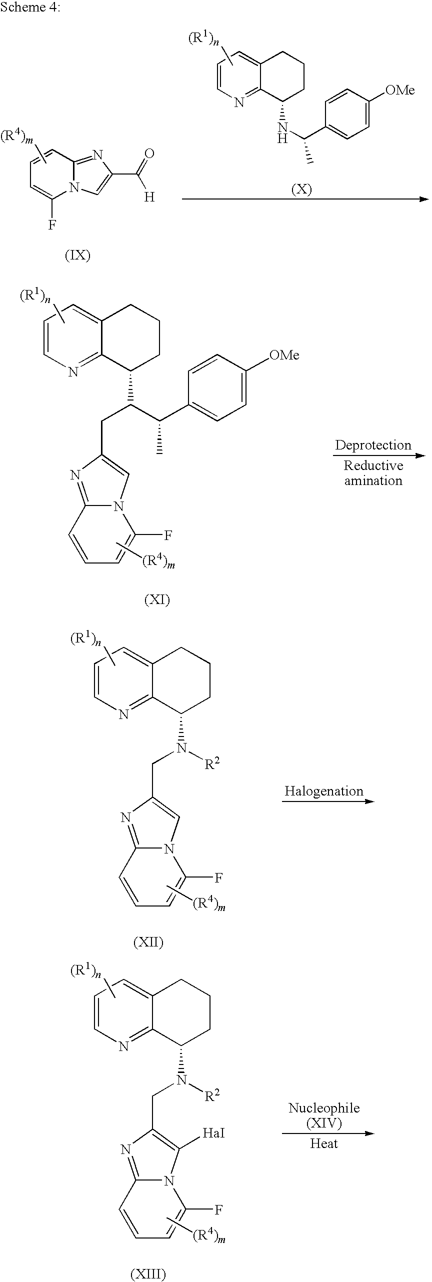

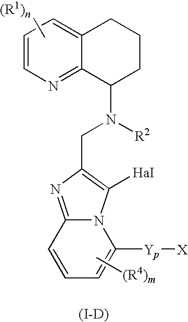

More specifically, compounds of formula (I-D) can be prepared by treating a compound of formula (XIII) with a nucleophile (XIV). The reaction can be carried out by treating the compound of formula (XIII) with a suitable nucleophile, neat, or optionally in the presence of an inert solvent. The reaction may be heated to 50-200° C. or performed at ambient temperature. Optionally the reaction may be carried out in a microwave. Suitable solvents include DMSO, N,N-dimethylformamide, alcohols or ethers (diglyme and the like).

Compounds of formula (XIII) can be prepared from compounds of formula (XII) by electrophilic halogenation. Suitable halogenating reagents include N-chlorosuccinimide, N-bromosuccinimide, N-iodosuccinimide and the like. Suitable solvents for the halogenation include alcohols (isopropanol and the like), tetrahydrofuran, dichloromethane, acetic acid and the like.

Compounds of formula (XII) can be prepared from a compound of formula (XI). Treatment of compound of formula (XI) with a stong acid in a suitable solvent is an appropriate deprotection method. Suitable acids include trifluoroacetic acid and the like. Suitable solvents include dichloromethane, dichloroethane and the like. The reaction can optionally be heated. Alternative deprotection methods include use of Lewis acids (e.g. BCl₃, AlCl₃, BBr₃ and the like) or removal of the protecting group under reductive conditions (e.g. Pd on charcoal or PtO₂ under H₂ atmosphere).

The resulting amine (compound of formula I wherein R² is H) can then be treated with a suitable aldehyde under reductive amination conditions to give a compound of formula (XII). The reductive amination can be carried out by treating the amine with the aldehyde in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

Compounds of formula (XI) can be prepared from compounds of formula (IX) and a compound of formula (X) by reductive amination. Aldehydes of formula (IX) can be prepared by methods similar to those described in the literature (e.g. J. Heterocyclic Chemistry, 1992, 29, 691-697, incorporated by reference with regard to such synthesis). Compound of formula (X) can be prepared form (S)-(−)-1-(4-methoxyphenyl)ethylamine and 6,7-dihydro-8(5H)-quinolinone (J. Org. Chem., 2002, 67, 2197-2205, herein incorporated by reference as it relates to such method) by reductive amination.

### EXAMPLES

### Example 1

### 5-Fluoroimidazo[1,2-a]pyridine-2-carbaldehyde (Intermediate)

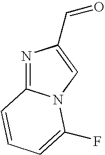

To a solution of 6-fluoro-2-pyridinamine (Tetrahedron, 2002, 58, 489, incorporated by reference with regard to such) (2.8 g, 25 mmol) in ethylene glycol dimethyl ether (28 mL) was added trichloroacetone (7.9 mL, 75 mmol). The mixture was stirred at room temperature for 15 hours and the resulting precipitate was collected by filtration and refluxed in ethyl alcohol (8 mL) for 4 hours. The reaction mixture was cooled to room temperature, concentrated, dissolved in dichloromethane and washed with saturated aqueous sodium bicarbonate. The organic layer was isolated, dried with magnesium sulfate, and concentrated. The resulting solid was refluxed in aqueous calcium carbonate for 2 hours, cooled to room temperature, and extracted with dichloromethane. The organic layer was dried with magnesium sulfate and concentrated to give 1.4 g (34% yield) 5-fluoroimidazo[1,2-a]pyridine-2-carbaldehyde as a tan solid. ¹H-NMR (CDCl₃): δ 10.16 (s, 1H), 8.22 (s, 1H), 7.54 (d, 1H), 7.34 (m, 1H), 6.59 (m, 1H); TLC (10% 2 M ammonia in methyl alcohol-ethyl acetate) R_{f}=0.60.

### Example 2

### 5-Bromoimidazo[1,2-a]pyridine-2-carbaldehyde (Intermediate)

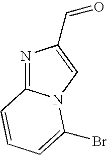

To a solution of 2-amino-6-bromopyridine (10 g, 58 mmol) in ethylene glycol dimethyl ether (66 mL) was added trichloroacetone (18 mL, 173 mmol). The mixture was stirred at 70° C. for 15 hours and the resulting precipitate was collected by filtration and refluxed in ethyl alcohol (50 mL) for 7 hours. The reaction mixture was cooled to room temperature, concentrated, dissolved in dichloromethane, and washed with saturated aqueous sodium bicarbonate. The organic layer was isolated, dried with magnesium sulfate, and concentrated. The resulting solid was refluxed in aqueous calcium carbonate for 1.5 hours, cooled to room temperature, and extracted with dichloromethane. The organic layer was dried with magnesium sulfate and concentrated to give 6.6 g (50% yield) 5-bromoimidazo[1,2-a]pyridine-2-carbaldehyde as an orange solid. ¹H-NMR (CDCl₃): δ 10.16 (s, 1H), 8.37 (s, 1H), 7.69 (d, 1H), 7.22 (m, 1H), 7.16 (m, 1H); TLC (10% ammonium hydroxide-acetonitrile) R_{f}=0.44.

### Example 3

### N-Methyl-5,6,7,8-tetrahydro-8-quinolinamine (Intermediate)

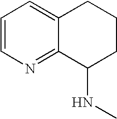

To a solution of 6,7-dihydro-8(5H)-quinolinone included in general processes above (1.5 g, 10 mmol) in dichloroethane (50 mL) was added methyl amine (2 M in tetrahydrofuran, 10 mL, 20 mmol), acetic acid (580 μL, 10 mmol), and sodium triacetoxyborohydride (4.3 g, 20 mmol). The mixture was stirred at room temperature for 15 hours and then filtered through a silica plug and rinsed with 10% ammonium hydroxide-acetonitrile. The solvent was removed and the residue purified by flash chromatography (0-10% ammonium hydroxide-acetonitrile) to give 1.4 g (85% yield) N-methyl-5,6,7,8-tetrahydro-8-quinolinamine as a yellow oil. ¹H-NMR (CDCl₃): δ 8.37 (d, 1H), 7.36 (d, 1H), 7.05 (t, 1H), 3.64 (t, 1H), 2.75 (m, 2H), 2.52 (s, 3H), 2.11 (m, 1H), 1.96 (m, 1H), 1.75 (m, 2H); MS m/z 163 (M+1).

### Example 4

### N-[(5-Fluoroimidazo[1,2-a]pyridin-2-yl)methyl]-N-methyl-5,6,7,8-tetrahydro-8-quinolinamine (Intermediate)

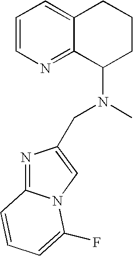

To a solution of N-methyl-5,6,7,8-tetrahydro-8-quinolinamine (340 mg, 2.1 mmol) and 5-fluoroimidazo[1,2-a]pyridine-2-carbaldehyde (344 mg, 2.1 mmol) in dichloroethane (10 mL) was added acetic acid (120 μL, 2.1 mmol) and sodium triacetoxyborohydride (1.3 g, 6.3 mmol). The mixture was stirred at room temperature for 2 hours and then filtered through a silica plug and rinsed with 10% ammonium hydroxide-acetonitrile. The solvent was removed and the residue purified by flash chromatography (0-10% ammonium hydroxide-acetonitrile) to give 0.6 g (93% yield) N-[(5-fluoroimidazo[1,2-a]pyridin-2-yl)methyl]-N-methyl-5,6,7,8-tetrahydro-8-quinolinamine as a tan solid. ¹H-NMR (CDCl₃): δ 8.53 (d, 1H), 7.80 (s, 1H), 7.36 (m, 2H), 7.13 (m, 1H), 7.06 (m, 1H), 6.40 (m, 1H), 4.10 (m, 1H), 3.94 (s, 2H), 2.75 (m, 2H), 2.43 (s, 3H), 2.03 (m, 3H), 1.70 (m, 1H); MS m/z 311 (M+1).

### Example 5

### N-[(5-Bromoimidazo[1,2-a]pyridin-2-yl)methyl]-N-methyl-5,6,7,8-tetrahydro-8-quinolinamine (Intermediate)

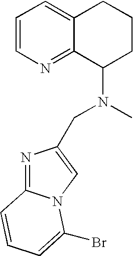

To a solution of N-methyl-5,6,7,8-tetrahydro-8-quinolinamine (500 mg, 3.1 mmol) and 5-bromoimidazo[1,2-a]pyridine-2-carbaldehyde (770 mg, 3.4 mmol) in dichloroethane (17 mL) was added acetic acid (180 μL, 3.1 mmol) and sodium triacetoxyborohydride (2.0 g, 9.3 mmol). The mixture was stirred at room temperature for 15 hours and then filtered through a silica plug and rinsed with 10% ammonium hydroxide-acetonitrile. The solvent was removed and the residue diluted with dichloromethane, washed with saturated aqueous sodium bicarbonate, and dried with magnesium sulfate to give 1.1 g (99% yield) of N-[(5-bromoimidazo[1,2-a]pyridin-2-yl)methyl]-N-methyl-5,6,7,8-tetrahydro-8-quinolinamine as an orange oil. ¹H-NMR (CDCl₃): δ 8.50 (d, 1H), 7.92 (s, 1H), 7.49 (d, 1H), 7.32 (d, 1H), 7.03 (m, 2H), 6.96 (m, 1H), 4.09 (m, 1H), 3.94 (s, 2H), 2.72 (m, 2H), 2.40 (s, 3H), 2.12 (m, 1H), 1.99 (m, 2H), 1.68 (m, 1H); MS m/z 372 (M+1).

### Example 6

### N-Methyl-N-{[5-(4-methyl-1-piperazinyl)imidazo[1,2-a]pyridin-2-yl]methyl}-5,6,7,8-tetrahydro-8-quinolinamine (Intermediate)

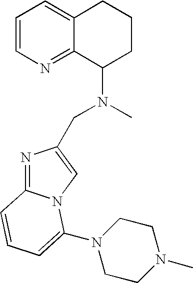

A solution of N-[(5-fluoroimidazo[1,2-a]pyridin-2-yl)methyl]-N-methyl-5,6,7,8-tetrahydro-8-quinolinamine (150 mg, 0.48 mmol) in neat 1-methylpiperazine (270 μL, 2.4 mmol) was subjected to microwave irradiation at 200° C. for 20 minutes. The reaction mixture was concentrated and purified by preparative chromatography (0-30% acetonitrile-water; 0.1% trifluoroacetic acid) and then diluted with ethyl acetate, washed with saturated aqueous sodium bicarbonate, and dried with magnesium sulfate to give 125 mg (67% yield) of a yellow oil. ¹H-NMR (CDCl₃): δ 8.52 (d, 1H), 7.70 (s, 1H), 7.34 (m, 1H), 7.28 (m, 1H), 7.10 (m, 1H), 7.04 (m, 1H), 6.23 (dd, 1H), 4.13 (m, 1H), 3.96 (m, 2H), 3.13 (s, 4H), 2.82 (m, 2H), 2.65 (s, 4H), 2.40 (s, 6H), 2.01 (m, 3H), 1.70 (m, 1H); MS m/z 391 (M+1).

Alternatively:

N-Methyl-N-{[5-(4-methyl-1-piperazinyl)imidazo[1,2-a]pyridin-2-yl]methyl}-5,6,7,8-tetrahydro-8-quinolinamine can also be prepared by reductive amination. A solution of N-methyl-5,6,7,8-tetrahydro-8-quinolinamine (44 mg, 0.27 mmol) and 5-(4-methyl-1-piperazinyl)imidazo[1,2-a]pyridine-2-carbaldehyde (69 mg, 0.30 mmol) in dichloroethane (1.4 mL) was treated with glacial acetic acid (15 μL, 0.27 mmol) and sodium triacetoxyborohydride (172 mg, 0.81 mmol). The mixture was stirred at room temperature for 15 hours and then filtered through a silica plug and rinsed with 10% 2M ammonia in methanol-ethyl acetate. The reaction mixture was concentrated and purified by preparative chromatography (0-70% acetonitrile-water; 0.1% trifluoroacetic acid) and then diluted with ethyl acetate, washed with saturated aqueous sodium bicarbonate, and dried with magnesium sulfate to give 9 mg (9% yield) of a yellow oil.

This racemic compound can also be separated by SFC to give the R and S isomers. Racemic N-methyl-N-{[5-(4-methyl-1-piperazinyl)imidazo[1,2-a]pyridin-2-yl]methyl}-5,6,7,8-tetrahydro-8-quinolinamine was separated into R and S isomers on a Berger analytical SFC with an HP1100 diode array detector. The sample was monitored at 230 nm under the following conditions: 15% co-solvent (50/50 MeOH/CHCl₃ with 0.5% diisopropylethylamine v/v) in CO₂ with a total flow rate of 2 mL/minute at 1500 psi, 27° C. on a Diacel AD-H column (Chiral Technologies), 4.6×250 mm, 5 um.

### Example 7

### (5-(4-Methyl-1-piperazinyl)-2-{[methyl(5,6,7,8-tetrahydro-8-quinolinyl)amino]methyl}imidazo[1,2-a]pyridin-3-yl)methanol (Intermediate)

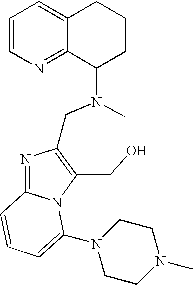

(5-(4-Methyl-1-piperazinyl)-2-{[methyl(5,6,7,8-tetrahydro-8-quinolinyl)amino]methyl}imidazo[1,2-a]pyridin-3-yl)methanol was prepared from N-methyl-N-{[5-(4-methyl-1-piperazinyl)imidazo[1,2-a]pyridin-2-yl]methyl}-5,6,7,8-tetrahydro-8-quinolinamine via hydroxymethylation in a similar manner as shown herein to give a white solid (30% yield). ¹H NMR (400 MHz, CDCl₃) δ 8.42 (m, 1H), 7.33-7.29 (m, 2H), 7.07-6.99 (m, 2H), 6.39 (d, J=7.1 Hz, 1H), 5.28 (s, 2H), 4.08-3.95 (m, 3H), 3.51 (d, J=10.1 Hz, 1H), 3.37 (d, J=10.6 Hz, 1H), 2.89 (m, 4H), 2.76 (m, 1H), 2.66 (m, 1H), 2.55-2.47 (m, 2H), 2.39 (s, 3H), 2.21 (m, 1H), 2.13 (s, 3H), 2.02-1.88 (m, 2H), 1.67 (m, 1H); MS m/z 421 (M+1).

### Example 8

### (8S)—N-{(1S)-1-[4-(Methyloxy)phenyl]ethyl}-N-{[5-(4-methyl-1-piperazinyl)imidazo[1,2-a]pyridin-2-yl]methyl}-5,6,7,8-tetrahydro-8-quinolinamine (Intermediate)

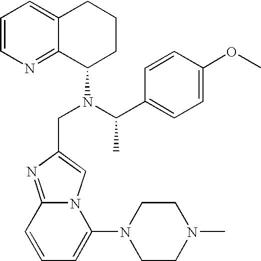

### A) 6-Fluoro-2-pyridinamine

A solution of 2,6-difluoropyridine (50 g, 434 mmol) in ammonium hydroxide (200 mL, 28.0-30.0%) was heated at 105° C. in a steel bomb for 15 hours. The reaction was cooled in an ice bath and the precipitate filtered, rinsed with cold water, and dried to yield 6-fluoro-2-pyridinamine (45.8 g, 94% yield) as a white solid. ¹H-NMR (CDCl₃): δ 7.53 (m, 1H), 6.36 (dd, 1H), 6.26 (dd, 1H), 4.56 (s, 2H).

### B) 2-(Dichloromethyl)-5-fluoroimidazo[1,2-a]pyridine

A solution of 6-fluoro-2-pyridinamine (67 g, 0.60 mol) in ethylene glycol dimethyl ether (570 mL) was treated with 1,1,3-trichloroacetone (190 mL, 1.80 mol) and heated at 85° C. for 15 hours. The reaction was cooled in an ice bath and the precipitate filtered, rinsed with hexanes, and dried to yield 2-(dichloromethyl)-5-fluoroimidazo[1,2-a]pyridine (85 g, 65% yield) as an olive green solid. ¹H-NMR (CDCl₃): δ 8.18 (s, 1H), 7.60 (s, 1H), 7.54-7.46 (m, 2H), 6.93 (m, 1H).

### C) 5-Fluoroimidazo[1,2-a]pyridine-2-carbaldehyde

A solution of 2-(dichloromethyl)-5-fluoroimidazo[1,2-a]pyridine (103 g, 470 mmol) in ethanol (300 mL) and water (600 mL) was treated with sodium acetate (96 g, 1.17 mol) and heated at 60° C. for 2 hours. The reaction was cooled, filtered though celite, and concentrated in vacuo to remove the ethanol. The aqueous was extracted twice with chloroform and the organics were combined, washed with water and brine, dried over sodium sulfate, and concentrated. The residue was filtered through a pad of silica, rinsed with dichloromethane and ethyl acetate, concentrated, triturated with hexanes, filtered, and dried to yield 5-fluoroimidazo[1,2-a]pyridine-2-carbaldehyde (40 g, 52% yield) as a tan solid. ¹H-NMR (CDCl₃): δ 10.17 (s, 1H), 8.22 (s, 1H), 7.57 (d, 1H), 7.38-7.32 (m, 1H), 6.60 (m, 1H); TLC (10% 2 M ammonia in methy alcohol-ethyl acetate) R_{f}=0.60.

### D) (5-Fluoroimidazo[1,2-a]pyridin-2-yl)methanol

A solution of 5-fluoroimidazo[1,2-a]pyridine-2-carbaldehyde (80 g, 490 mmol) in methanol (1 L) at 0° C. was treated with sodium borohydride (24 g, 640 mmol) in portions. The reaction was slowly brought to room temperature, stirred for 2 hours, quenched with water, concentrated, dissolved in 3:1 dichloromethane to isopropyl alcohol, and washed with saturated aqueous sodium bicarbonate. The organic layer was separated and the aqueous extracted four times with 3:1 dichloromethane to isopropyl alcohol. The organic layers were combined, dried over sodium sulfate, concentrated, triturated with hexanes, and filtered to yield (5-fluoroimidazo[1,2-a]pyridin-2-yl)methanol (76 g, 93% yield) as a brown solid. ¹H-NMR (CDCl₃): δ 7.59 (s, 1H), 7.38 (d, 1H), 7.21-7.15 (m, 1H), 6.43 (m, 1H), 4.85 (s, 2H), 4.45 (s, 1H).

### E) [5-(4-Methyl-1-piperazinyl)imidazo[1,2-a]pyridin-2-yl]methanol

A solution of (5-fluoroimidazo[1,2-a]pyridin-2-yl)methanol (76 g, 460 mmol) in 1-methyl piperazine (150 mL) was heated at 70° C. for 15 hours. The reaction mixture was cooled, poured into 1.3 L brine, and extracted into 3:1 chloroform to isopropyl alcohol. The combined extracts were dried over sodium sulfate, concentrated in vacuo, azeotroped with hexanes, and triturated with diethyl ether to yield [5-(4-methyl-1-piperazinyl)imidazo[1,2-a]pyridin-2-yl]methanol (101 g, 90% yield) as a tan solid. ¹H-NMR (CDCl₃): δ 7.51 (s, 1H), 7.33 (d, 1H), 7.21-7.17 (m, 1H), 6.31 (m, 1H), 4.87 (s, 2H), 3.17 (s, 4H), 2.68 (s, 4H), 2.42 (s, 3H).

### F) 5-(4-Methyl-1-piperazinyl)imidazo[1,2-a]pyridine-2-carbaldehyde

A solution of [5-(4-methyl-1-piperazinyl)imidazo[1,2-a]pyridin-2-yl]methanol (101 g, 410 mmol) in chloroform (1650 mL) was treated with manganese dioxide (360 g, 4100 mmol) and stirred at room temperature for 72 hours. The reaction mixture was filtered through celite, rinsed with chloroform, and concentrated to yield 5-(4-methyl-1-piperazinyl)imidazo[1,2-a]pyridine-2-carbaldehyde (82 g, 82% yield) as gold solid. ¹H-NMR (CDCl₃): δ 10.17 (s, 1H), 8.15 (s, 1H), 7.44 (d, 1H), 7.31-7.27 (m, 1H), 6.40 (m, 1H), 3.16 (s, 4H), 2.68 (s, 4H), 2.42 (s, 3H).

### G) (8S)—N-{(1S)-1-[4-(Methyloxy)phenyl]ethyl}-5,6,7,8-tetrahydro-8-quinolinamine

A solution of (S)-(−)-1-(4-methoxyphenyl)ethylamine (25 g, 166 mmol) and 6,7-dihydro-8(5H)-quinolinone (24 g, 166 mmol) in dichloromethane was treated with glacial acetic acid (14 mL, 249 mmol) and sodium triacetoxyborohydride (53 g, 249 mmol). The reaction mixture was stirred at room temperature for 15 hours and treated with sodium carbonate (106 g, 996 mmol) and stirred for 30 minutes. The mixture was diluted with dichloromethane, the organic layer separated, and the aqueous extracted with more dichloromethane. The organic layers were combined, dried over magnesium sulfate, concentrated, and purified by column chromatography (0-3% 2 M ammonia in methanol/dichloromethane) to give a yellow oil which was crystallized from hexanes to yield (8S)—N-{(1S)-1-[4-(methyloxy)phenyl]ethyl}-5,6,7,8-tetrahydro-8-quinolinamine (33 g, 70% yield) as clear crystals. ¹H-NMR (CDCl₃): δ 8.40 (m, 1H), 7.33 (m, 3H), 7.04 (m, 1H), 6.84 (d, 2H), 4.02 (m, 1H), 3.83-3.78 (m, 4H), 2.73-2.62 (m, 2H), 1.82 (m, 1H), 1.72 (m, 1H), 1.57 (m, 2H), 1.43 (d, 3H).

### H) (8S)—N-{(1S)-1-[4-(Methyloxy)phenyl]ethyl}-N-{[5-[4-methyl-1-piperazinyl)imidazol-1,2-a]pyridin-2-yl]methyl}-5,6,7,8-tetrahydro-8-quinolinamine

A solution of 5-(4-methyl-1-piperazinyl)imidazo[1,2-a]pyridine-2-carbaldehyde (2.83 g, 11.6 mmol) and (8S)—N-{(1S)-1-[4-(methyloxy)phenyl]ethyl}-5,6,7,8-tetrahydro-8-quinolinamine (3.27 g, 11.6 mmol) in dichloroethane (40 mL) was treated with glacial acetic acid (1.0 mL, 17.4 mmol) and sodium triacetoxyborohydride (3.68 g, 17.4 mmol, added in portions) and stirred at room temperature for 15 hours. The reaction mixture was diluted with dichloromethane, washed with saturated aqueous sodium bicarbonate, separated, and extracted with additional dichloromethane. The organic layers were combined, washed with brine, dried over sodium sulfate, concentrated, and purified by flash chromatography (0-4% ammonium hydroxide in acetonitrile). The residue was dissolved in dichloromethane and stirred with 2 M ammonia in methanol to yield (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine (5.13 g, 87% yield) as pale yellow foam.

¹H NMR (400 MHz, CDCl₃) δ 8.48 (d, J=4.6 Hz, 1H), 7.78 (s, 1H), 7.60-7.58 (m, 2H), 7.24-7.18 (m, 2H), 7.09-7.05 (m, 1H), 6.97 (dd, J=7.6, 4.7 Hz, 1H), 6.84-6.82 (m, 2H), 6.21 (d, J=7.2 Hz, 1H), 4.82 (m, 1H), 4.07 (m, 1H), 3.91 (dd, J=56.9, 17.1 Hz, 2H), 3.77 (s, 3H), 3.19-3.13 (m, 4H), 2.74 (s, 4H), 2.67-2.53 (m, 2H), 2.47 (s, 3H), 2.06 (m, 1H), 1.85 (m, 2H), 1.53 (m, 1H), 1.34 (d, J=6.4 Hz, 3H); MS m/z 511 (M+1).

### Example 9

### (8S)—N-{[5-(4-methyl-1-piperazinyl)imidazo[1,2-a]pyridin-2-yl]methyl}-5,6,7,8-tetrahydro-8-quinolinamine (Intermediate)

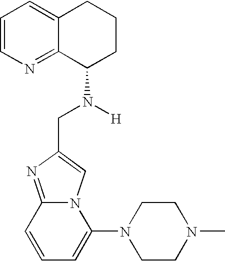

A solution of (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine (569 mg, 1.11 mmol) in dichloromethane (11.1 mL) was treated with trifluoroacetic acid (1.11 mL) and stirred at room temperature for 4 hours. The reaction was concentrated, diluted with dichloromethane, and washed with saturated aqueous sodium bicarbonate. The organic layer was separated and the aqueous extracted with dichloromethane. The organic layers were combined, dried over magnesium sulfate, filtered, and concentrated to yield (8S)—N-{[5-(4-methyl-1-piperazinyl)imidazo[1,2-a]pyridin-2-yl]methyl}-5,6,7,8-tetrahydro-8-quinolinamine as a yellow residue. ¹H-NMR (CDCl₃): δ 8.41 (d, 1H), 7.65 (s, 1H), 7.39 (d, 1H), 7.31 (m, 1H), 7.16 (m, 1H), 7.09 (m, 1H), 6.27 (dd, 1H), 4.31-4.17 (m, 2H), 4.05 (m, 1H), 3.15 (m, 4H), 2.88-2.78 (m, 2H), 2.67 (m, 4H), 2.41 (s, 3H), 2.29 (m, 1H), 2.08 (m, 1H), 1.96 (m, 1H), 1.77 (m, 1H).

### Example 10

### (8S)—N-Methyl-N-{[5-(4-methyl-1-piperazinyl)imidazo[1,2-a]pyridin-2-yl]methyl}-5,6,7,8-tetrahydro-8-quinolinamine (Intermediate)

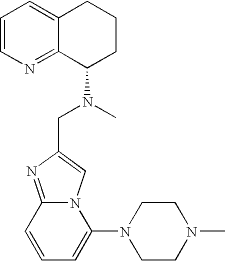

(8S)—N-{[5-(4-methyl-1-piperazinyl)imidazo[1,2-a]pyridin-2-yl]methyl}-5,6,7,8-tetrahydro-8-quinolinamine was dissolved in dichloroethane (10 mL) and treated with formaldehyde (166 μL, 2.22 mmol, 37 wt. % solution in water), glacial acetic acid (96 μL, 1.67 mmol), sodium triacetoxyborohydride (353 mg, 1.67 mmol) and stirred at room temperature for 15 hours. The reaction was diluted with dichloromethane and washed with saturated aqueous sodium bicarbonate. The organic layer was separated and the aqueous extracted with dichloromethane. The organic layers were combined, dried over magnesium sulfate, filtered, concentrated, and purified by flash chromatography (0-10% ammonium hydroxide in acetonitrile) to give 0.276 g (64% yield from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine) (8S)—N-methyl-N-{[5-(4-methyl-1-piperazinyl)imidazo[1,2-a]pyridin-2-yl]methyl}-5,6,7,8-tetrahydro-8-quinolinamine as a pale yellow oil. ¹H-NMR (CDCl₃): δ 8.52 (d, 1H), 7.70 (s, 1H), 7.34 (d, 1H), 7.28 (d, 1H), 7.10 (m, 1H), 7.06 (m, 1H), 6.23 (dd, 1H), 4.12 (m, 1H), 3.96 (s, 2H), 3.14 (m, 4H), 2.86-2.78 (m, 2H), 2.71-2.65 (m, 4H), 2.41 (5, 3H), 2.39 (s, 3H), 2.16 (m, 1H), 2.06-1.97 (m, 2H), 1.68 (m, 1H); MS m/z 391 (M+1). ¹³C-NMR (CDCl₃): δ 158.0, 147.0, 146.2, 145.5, 145.2, 136.4, 134.1, 124.7, 121.4, 111.9, 107.9, 98.9, 62.5, 55.0, 55.0, 53.1, 49.5, 49.5, 46.1, 39.0, 29.2, 24.2, 21.1; HRMS: Calculated Mass: 391.2610; Found Mass: 391.2614; Formula: C₂₃H₃₁N₆. Analyis Calculated for C₂₃H₃₀N₆: C, 70.74; H, 7.74; N, 21.52. Found: C, 70.36; H, 7.77; N, 21.53.

Alternatively (8S)—N-methyl-N-{[5-(4-methyl-1-piperazinyl)imidazo[1,2-a]pyridin-2-yl]methyl}-5,6,7,8-tetrahydro-8-quinolinamine can be synthesized in the following fashion:

### A) 5-bromoimidazo[1,2-a]pyridine-2-carbaldehyde

The reactor is charged with 2-amino-6-bromopyridine (3.0 Kg, 17.3 mol) and dimethoxyethane (12 Liters) and stirred under nitrogen. 1,1,3-Trichloroacetone (5.6 Kg, 30.3 mol) is added to the 25° C. solution in a single portion and the reaction solution is warmed to 65° C. jacket temperature and maintained for approximately 2 to 4 hours until judged complete. The reaction is cooled to 10° C. and held for approximately one hour and filtered. The solids are rinsed with dimethoxyethane (6 Liters). The solid is placed back in the reactor and treated with dimethoxyethane (12 Liters) and 2N HCl (12 Liters) and warmed to aproximately 75 degrees for 16 to 20 hours or until judged complete. The reaction is cooled to approximately 10° C. and pH is adjusted to approximately 8 with 3 N NaOH. The resulting solids are filtered and washed with water. The solid is dried at 50° C. for 16 hours to yield 5-bromoimidazo[1,2-a]pyridine-2-carbaldehyde, (2.81 Kg, 72% yield) ¹H NMR (400 MHz, DMSO-D6) δ ppm 10.05 (s, 1H) 8.66 (s, 1H) 7.72 (s, 1H) 7.42 (s, 1H) 7.35 (s, 1H)

### B) 5-(4-methyl-1-piperazinyl)imidazo[1,2-a]pyridine-2-carbaldehyde

The reactor is charged with N-methylpiperazine (3.1 Kg, 31 mol) and tetrahydrofuran (10 Liters) and stirred under nitrogen while cooling to negative 20° C. n-Butyl lithium (10.4 L, 26.0 mol) is added to the reaction at a rate to maintain the negative 20° C. temp and the contents are stirred for 15 to 30 minutes. A slurry of 5-bromoimidazo[1,2-a]pyridine-2-carbaldehyde (2.79 Kg, 12.4 mol) in tetrahydrofuran (10 Liters) is added at a rate to maintain the reaction at 50° C. The slurry is washed in with additional tetrahydrofuran (6 Liters). The reaction is stirred for 30 minutes and warmed to approximately negative 10° C. The reaction is quenched by addition of 6N HCl solution to achieve pH 4.0 while maintaining at 515° C. The reaction is diluted with heptane (14 Liters) and the layers allowed to separate. The lower aqueous layer is drained and the upper organic layer is washed with 1N HCl (2×1.5 Liters). The combined aqueous layers are stirred at 20 degrees and adjusted to pH 9 with 4N NaOH solution. The Aqueous layer is extracted with 10% iPrOH/CH₂Cl₂ (3×28 Liters) and the combined organic layers are washed with saturated NaHCO₃ solution (14 Liters) and evaporated at <25° C. to approximately 3 volumes. Isopropanol (28 Liters) is added and reaction again concentrated under reduced pressure to approximately 8.5 Liters. Isopropanol (17 Liters) is added and the reaction is treated with a solution of oxalic acid (1.0 Kg, 11.1 mol) in isopropanol (7 Liters) at a rate to maintain good stirring and temperature between approximately 25-40° C. The reaction is stirred for 30 minutes and the solids are collected and washed with isopropanol (8.5 Liters) Solids are dried at 50° C. to yield 5-(4-methyl-1-piperazinyl)imidazo[1,2-a]pyridine-2-carbaldehyde, (2.25 Kg, 54% yield) ¹H NMR (400 MHz, DMSO-D6) δ ppm 10.01 (s, 1H) 8.47 (s, 1H) 7.41 (m, 2H) 6.65 (m, 1H) 3.34 (s, 8H) 2.78 (s, 3H)

### C) (8S)—N-{(1S)-1-[4-(methyloxy)phenyl]ethyl}-5,6,7,8-tetrahydro-8-quinolinamine

A slurry of sodium triacetoxyborohydride (4.54 Kg, 21.4 mol.) in dichloromethane (22 Liters) is treated with 6,7-dihydro-8(5H)-quinolinone (1.8 Kg, 12.3 mol.) followed by (1S)-1-[4-(methyloxy)phenyl]ethanamine (1.8 Kg, 11.9 mol).) and the reaction was allowed to stirr vigorously at 22° C. for 24 hrs. The reaction is quenched with 1 N NaOH (aprox 27 Liters) to achieve pH 8 in the aqueous layer. The phases were separated and the organic phase was treated with 1N sodium hydroxide (aprox 3.5 Liters) to achieve pH 11 in the aqueous layer. The phases again separated. The dichloromethane solution was then concentrated to minimum volume and treated with heptane (18 Liters). The volume again concentrated to aprox 9 Liters. Precipitation occurred upon cooling to 22° C. The suspension was further cooled to 0° C. and filtered. Solids were dried at ambient temperature under vacuum with nitrogen to give (8S)—N-{(1S)-1-[4-(methyloxy)phenyl]ethyl}-5,6,7,8-tetrahydro-8-quinolinamine. (2.18 Kg, 63%) ¹H NMR (400 MHz, DMSO-D6) δ ppm 8.36 (m, 1H) 7.44 (m, 1H) 7.29 (m, 2H) 7.15 (m, 1H) 6.83 (m, 2H) 4.00 (m, 1H) 3.70 (s, 3H) 3.59-3.64 (m, 1H) 2.66 (m, 1H) 2.64 (s, 1H) 2.53 (s, 1H) 1.76 (s, 1H) 1.64 (s, 1H) 1.50 (s, 1H) 1.39 (s, 1H) 1.24 (m, 3H)

### D) (8S)—N-methyl-5,6,7,8-tetrahydro-8-quinolinamine

A slurry of sodium triacetoxyborohydride (2.44 Kg, 11.5 molo) and (8S)—N-{(1S)-1-[4-(methyloxy)phenyl]ethyl}-5,6,7,8-tetrahydro-8-quinolinamine (2.17 Kg, 7.7 mol.) in dichloromethane (21.8 Liters) is cooled to 5° C. Formaldehyde solution (37 wt. % in water, 744 ml, 10 mol.) is added slowly to maintain the temperature below 25 deg C. The solution is stirred for 30 min at 22° C. The reaction is then quenched with trifluoroacetic acid (7.3 Liters, 95 mol.) added slowly. Upon completion of the addition, the reaction is warmed up to 30° C. and stirred for 16 hrs. Water (11 Liters) is added and the two phases separated. The aqueous phase is washed with dichloromethane (14 Liters) and the combined organic phases washed with water (2×5.5 Liters). The organic phase is discarded. The pH of the aqueous phase is raised to 8.5-9 by the addition of 6N NaOH and the aqueous layer extracted with dichloromethane (3×13 Liters). The dichloromethane is exchanged for isopropanol to achieve a final volume of aprox. 7 5 Liters. This solution is then treated with a solution of oxalic acid (588 g, 6.5 Mol.) in isopropanol (2.2 Liters) to induce precipitation. After stirring for 2 hours, the suspension filtered at 22° C. to and the solids dried at 22° C. to afford (8S)—N-methyl-5,6,7,8-tetrahydro-8-quinolinamine oxalate salt. (1.07 Kg, 55% yield) ¹H NMR (300 MHz, DMSO-D6) δ ppm 9.25 (br s, 1H) 8.52 (s, 1H) 7.69 (s, 1H) 7.39 (s, 1H) 4.39 (s, 1H) 2.82 (s, 2H) 2.65 (s, 3H) 2.50 (s, 1H) 2.32 (s, 1H) 1.99 (s, 1H) 1.80 (s, 1H);

For (8S)—N-methyl-5,6,7,8-tetrahydro-8-quinolinamine as free base: ¹H-NMR (CDCl₃): δ 8.37 (d, 1H), 7.36 (d, 1H), 7.06 (dd, 1H), 3.65 (m, 1H), 2.76 (m, 2H), 2.53 (s, 3H), 2.11 (m, 1H), 1.97 (m, 1H), 1.75 (m, 2H); MS m/z 163 (M+1).

### E) (8S)—N-methyl-N-{[5-(4-methyl-1-piperazinyl)imidazo[1,2-a]pyridin-2-yl]methyl}-5,6,7,8-tetrahydro-8-quinolinamine

A slurry of sodium triacetoxyborohydride (0.63 g, 2.97 mmol) and (8S)—N-methyl-5,6,7,8-tetrahydro-8-quinolinamine. (0.5 g, 1.98 mmol.) in DCM (50 ml.) is stirred at 20° C. To this is added 5-(4-methyl-1-piperazinyl)imidazo[1,2-a]pyridine-2-carbaldehyde oxylate (0.84 g, 2.97 mmol.) and the reaction is allowed to stir at 20° C. for 16 hours. The reaction was then quenched with 2N NaOH to achieve pH 12 and the layers allowed to separate. The aqueous layer is washed with additional dichloromethane (3×10 ml) and the combined organic layers were evaporated to oil that was dried under high vacuum to give (8S)—N-methyl-N-{[5-(4-methyl-1-piperazinyl)imidazo[1,2-a]pyridin-2-yl]methyl}-5,6,7,8-tetrahydro-8-quinolinamine as a tan oil (0.6 g, 77%) ¹H NMR compares to above.

### Example 11

### [5-(4-Methyl-1-piperazinyl)-2-({methyl[(8S)-5,6,7,8-tetrahydro-8-quinolinyl]amino}methyl)imidazo[1,2-a]pyridin-3-yl]methanol (Intermediate)

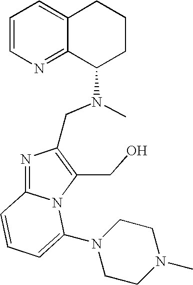

A solution of (8S)—N-methyl-N-{[5-(4-methyl-1-piperazinyl)imidazo[1,2-a]pyridin-2-yl]methyl}-5,6,7,8-tetrahydro-8-quinolinamine (2.9 g, 7.4 mmol) in formaldehyde (10 mL, 37 wt. % solution in water) and glacial acetic acid (2.5 mL) was heated at 50° C. for 15 hours. The reaction mixture was cooled, diluted with dichloromethane, and washed with saturated aqueous sodium carbonate. The organic layer was isolated and the aqueous washed three times with dichloromethane/isopropyl alcohol. The organic layers were combined, dried with magnesium sulfate, filtered, and concentrated. The residue was purified by flash chromatography (0-10% ammonium hydroxide in acetonitrile) to give 2.1 g (68% yield) [5-(4-methyl-1-piperazinyl)-2-({methyl[(8S)-5,6,7,8-tetrahydro-8-quinolinyl]amino}methyl)imidazo[1,2-a]pyridin-3-yl]methanol as a white solid. The solid was recrystallized from dichloromethane and hexanes. ¹H-NMR (CDCl₃): δ 8.42 (d, 1H), 7.31 (m, 2H), 7.06 (m, 1H), 7.01 (m, 1H), 6.75 (s, 1H), 6.39 (d, 1H), 5.29 (m, 2H), 4.01 (m, 3H), 3.52 (m, 1H), 3.38 (m, 1H), 2.90 (m, 4H), 2.78 (m, 1H), 2.67 (m, 1H), 2.52 (m, 2H), 2.40 (s, 3H), 2.21 (m, 1H), 2.13 (s, 3H), 1.96 (m, 2H), 1.68 (m, 1H); MS m/z 443 (M+Na)⁺. ¹³C-NMR (CDCl₃): δ 157.1, 148.4, 147.2, 146.2, 145.5, 136.9, 134.3, 125.2, 124.4, 121.7, 113.7, 102.0, 61.9, 55.11, 54.9, 54.0, 53.6, 51.9, 51.7, 46.3, 36.9, 29.4, 21.6, 21.5.

High Resolution MS: Calculated Mass: 421.2710; Found Mass: 421.2707; Formula: C₂₄H₃₃N₆O.

Analyis Calculated for C₂₄H₃₂N₆O: C, 68.54; H, 7.67; N, 19.98. Found: C, 68.26; H, 7.72; N, 19.89. Absolute stereochemistry confirmed by X-ray.

Alternatively, a slurry of sodium triacetoxyborohydride (1.86 Kg) and (8S)—N-methyl-5,6,7,8-tetrahydro-8-quinolinamine oxalate salt. (1.3 Kg, 5.15 mol.) in dichloromethane (13 Liters.) is stirred at 20° C. A solution of 5-(4-methyl-1-piperazinyl)imidazo[1,2-a]pyridine-2-carbaldehyde oxylate (2.07 Kg, 6.18 mol.) and triethylamine (1.25 Kg, 12.4 mol.) in dichloromethane (6.5 Liters) is added to the reaction at a rate to maintain the temperature belowe 30° C. The reaction is stirred at 20° C. for 16 hours. The reaction was then quenched with 2N NaOH to achieve pH 12 (Aprox 13 Liters). Methanol (aprox 6 Liters) is added to achieve a bilayer. The lower organic layer is separated and aqueous layer washed with dichloromethane (4×5 Liters). The combined organic layers were evaporated to minimum stir volume and the solvent was exchanged for water to achieve a final concenteration of 6.5 Liters. This solution was maintained at 40° C. and treated with 37% aqueous formaldehyde solution (2.7 Liters, 35 mol.). Solution allowed to stir at 40° C. for 24 hours and additional formaldehyde solution added (1.35 Liters, 18 mol). Reaction was allowed to stir for 72 hours and then cooled to 25° C. and treated with saturated aqueous sodium bicarbonate (5.2 Liters vol) and dichloromethane (6.5 Liters). The layers separated and the aqueous layer washed with additional dichloromethane (2×6.5 Liters). The combined organic layer washed with sodium bicarbonate solution (4 Liters) and then the organic layer filtered through a bed of silica gel 60 (3.9 Kg). The silica bed washed with additional dichloromethane (3×6.5 Liters) and the combined organic solvent was evaporated to minimum stir volume. Ethyl acetate (13 Liters) was added and the solvent again evaporated to a final concentration of 6.5 Liters. The solution was cooled slowly and crystallization occurred. The solids are filtered and rinsed with ethyl acetate (2.6 Liters). Solids dried at 45° C. to give [5-(4-methyl-1-piperazinyl)-2-({methyl[(8S)-5,6,7,8-tetrahydro-8-quinolinyl]amino}methyl)imidazo[1,2-a]pyridin-3-yl]methanol. (1.15 Kg, 53%) ¹H NMR (400 MHz, DMSO-D6) δ ppm 7.47 (s, 1H) 7.23 (s, 1H) 7.12 (s, 2H) 6.53 (s, 1H) 5.95 (s, 1H) 5.09 (s, 2H) 3.89 (s, 3H) 3.30 (s, 4H) 2.77 (s, 5H) 2.64 (s, 1H) 2.47 (s, 1H) 2.27 (s, 5H) 2.01 (s, 4H) 1.89 (s, 2H) 1.58 (s, 1H)

### Example 12

### 5-(4-Methyl-1-piperazinyl)-2-({methyl[(8S)-5,6,7,8-tetrahydro-8-quinolinyl]amino}methyl)imidazo[1,2-a]pyridine-3-carbaldehyde (Intermediate)

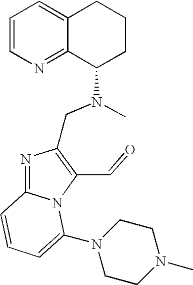

A solution of [5-(4-methyl-1-piperazinyl)-2-({methyl[(8S)-5,6,7,8-tetrahydro-8-quinolinyl]amino}methyl)imidazo[1,2-a]pyridin-3-yl]methanol (572 mg, 1.36 mmol) in dichloromethane (7 mL) was treated with IBX polystyrene (2 g, 2.8 mmol), stirred at room temperature for 15 hours, treated with additional IBX polystyrene (3 g, 4.2 mmol), and stirred at room temperature 24 hours. The reaction mixture was filtered, rinsed with dichloromethane, dissolved in methanol, heated at 40° C. for 15 hours, filtered, concentrated, and purified by flash chromatography (0-10% ammonium hydroxide in acetonitrile) to give 330 mg (58% yield) of 5-(4-methyl-1-piperazinyl)-2-({methyl[(8S)-5,6,7,8-tetrahydro-8-quinolinyl]amino}methyl)imidazo[1,2-a]pyridine-3-carbaldehyde as an orange oil. ¹H-NMR (CDCl₃): δ 10.86 (s, 1H), 8.47 (d, 1H), 7.47 (d, 1H), 7.36 (m, 2H), 7.04 (m, 1H), 6.61 (dd, 1H), 4.29 (s, 2H), 4.23 (m, 1H), 3.35 (m, 2H), 2.94-2.81 (m, 4H), 2.71-2.67 (m, 2H), 2.50 (s, 3H), 2.40 (m, 2H), 2.37 (s, 3H), 2.19 (m, 1H), 2.08 (m, 2H), 1.71 (m, 1H).

### Example 13

### 1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanol (Intermediate)

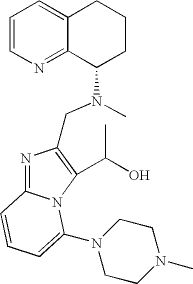

A solution of 5-(4-methyl-1-piperazinyl)-2-({methyl[(8S)-5,6,7,8-tetrahydro-8-quinolinyl]amino}methyl)imidazo[1,2-a]pyridine-3-carbaldehyde (51 mg, 0.12 mmol) in tetrahydrofuran (0.50 mL) at 0° C. was treated with methyl magnesium bromide (80 μL, 0.24 mmol), brought to room temperature, and stirred for 15 hours. The reaction was diluted with dichloromethane, washed with saturated aqueous sodium carbonate, separated, concentrated, and purified by flash chromatography (0-10% ammonium hydroxide in acetonitrile) to give 29 mg (56% yield) of 1-[5-(4-methyl-1-piperazinyl)-2-({methyl[(8S)-5,6,7,8-tetrahydro-8-quinolinyl]amino}methyl)imidazo[1,2-a]pyridin-3-yl]ethanol as a yellow solid. ¹H NMR (400 MHz, CDCl₃) δ 8.49-8.45 (m, 1H), 7.36-7.33 (m, 1H), 7.31-7.29 (m, 1H), 7.06-7.01 (m, 2H), 6.41 (d, J=6.9 Hz, 1H), 6.12-6.07 (m, 1H), 4.37 (m, 1H), 4.23-4.04 (m, 2H), 3.34 (m, 1H), 3.12-3.02 (m, 2H), 2.90-2.79 (m, 3H), 2.74-2.64 (m, 2H), 2.52 (m, 1H), 2.39-2.33 (m, 4H), 2.10-1.96 (m, 6H), 1.71 (m, 1H), 1.59-1.46 (m, 3H); MS m/z 435 (M+1).

### Example 14

### 1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanone

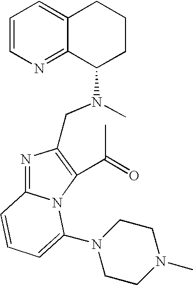

A solution of [115-(4-methyl-1-piperazinyl)-2-({methyl[(8S)-5,6,7,8-tetrahydro-8-quinolinyl]amino}methyl)imidazo[1,2-a]pyridin-3-yl]ethanol (25 mg, 0.058 mmol) in N,N-dimethylformamide (1 mL) was treated with IBX polystyrene (131 mg, 0.14 mmol, Novabiochem), stirred at room temperature for 15 hours, filtered, rinsed with dichloromethane, and concentrated to dryness under reduced pressure. The filtered resin was stirred in methanol at 40° C. for 15 hours, filtered, rinsed with dichloromethane, concentrated, and combined with the original filtrate. The crude material was purified by flash chromatography (0-10% ammonium hydroxide in acetonitrile) to give 5 mg (16% yield) 1-[5-(4-methyl-1-piperazinyl)-2-({methyl[(8S)-5,6,7,8-tetrahydro-8-quinolinyl]amino}methyl)imidazo[1,2-a]pyridin-3-yl]ethanone as a yellow oil. ¹H NMR (400 MHz, CDCl₃) δ 8.46 (m, 1H), 7.38-7.35 (m, 2H), 7.24-7.21 (m, 1H), 7.07-7.04 (m, 1H), 6.45 (d, 1H), 4.05-4.01 (m, 3H), 3.23 (m, 2H), 2.82-2.70 (m, 6H), 2.56 (s, 3H), 2.36 (s, 3H), 2.33 (s, 3H), 2.28 (m, 2H), 2.11 (m, 1H), 2.05-2.00 (m, 2H), 1.71 (m, 1H); MS m/z 433 (M+1).

### Example 15

### (8S)—N-[(3-Bromo-5-fluoroimidazo[1,2-a]pyridin-2-yl)methyl]-N-methyl-5,6,7,8-tetrahydro-8-quinolinamine (Intermediate)

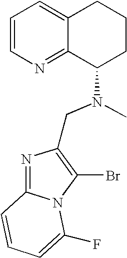

A solution of (8S)—N-[(5-fluoroimidazo[1,2-a]pyridin-2-yl)methyl]-N-methyl-5,6,7,8-tetrahydro-8-quinolinamine (114 mg, 0.37 mmol) in dichloromethane (2.5 mL) was treated with N-bromosuccinimide (98 mg, 0.55 mmol) and stirred at room temperature for 15 hours. The reaction was diluted with dichloromethane, washed with saturated aqueous sodium carbonate, separated, concentrated, and purified by flash chromatography (0-10% ammonium hydroxide in acetonitrile) to give 103 mg (30% yield) (8S)—N-[(3-bromo-5-fluoroimidazo[1,2-a]pyridin-2-yl)methyl]-N-methyl-5,6,7,8-tetrahydro-8-quinolinamine as a yellow oil. ¹H NMR (400 MHz, CDCl₃) δ 8.49 (d, 1H), 7.38 (d, 1H), 7.34 (d, 1H), 7.14-7.09 (m, 1H), 7.04 (dd, 1H), 6.39 (t, 1H), 4.06 (m, 1H), 3.90 (s, 2H), 2.88-2.81 (m, 1H), 2.73-2.66 (m, 1H), 2.40 (s, 3H), 2.12-2.05 (m, 3H), 1.70 (m, 1H); MS m/z 390 (M+1).

### Example 16

### (8S)—N-{[3-Bromo-5-(4-methyl-1-piperazinyl)imidazo[1,2-a]pyridin-2-yl]methyl}-N-methyl-5,6,7,8-tetrahydro-8-quinolinamine

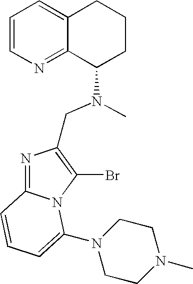

A solution of (8S)—N-[(3-bromo-5-fluoroimidazo[1,2-a]pyridin-2-yl)methyl]-N-methyl-5,6,7,8-tetrahydro-8-quinolinamine (30 mg, 0.077 mmol) in acetonitrile (1 mL) was treated with 1-methylpiperazine (43 μL, 0.39 mmol), heated at 50° C. for 15 hours, treated with additional 1-methylpiperazine (86 μL, 0.78 mmol), and heated at 50° C. for 72 hours. The reaction was diluted with dichloromethane, washed with saturated aqueous sodium carbonate, separated, concentrated, and purified by flash chromatography (0-10% ammonium hydroxide in acetonitrile) to give 8 mg (22% yield) (8S)—N-{[3-bromo-5-(4-methyl-1-piperazinyl)imidazo[1,2-a]pyridin-2-yl]methyl}-N-methyl-5,6,7,8-tetrahydro-8-quinolinamine as a red oil. ¹H NMR (400 MHz, CDCl₃) δ 8.49 (d, 1H), 7.35-7.31 (m, 2H), 7.10-7.02 (m, 2H), 7.04 (dd, 1H), 6.38-6.36 (m, 1H), 4.06 (m, 1H), 3.87 (5, 2H), 3.28-3.21 (m, 2H), 2.91-2.78 (m, 5H), 2.70-2.65 (m, 1H), 2.61-2.52 (m, 2H), 2.39 (s, 3H), 2.37 (s, 3H), 2.11-2.09 (m, 2H), 1.70 (m, 1H); MS m/z 470 (M+1).

### Example 17

### 5-(4-Methyl-1-piperazinyl)-2-({methyl[(8S)-5,6,7,8-tetrahydro-8-quinolinyl]amino}methyl)imidazo[1,2-a]pyridine-3-carbonitrile

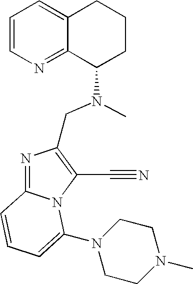

A solution of 5-(4-methyl-1-piperazinyl)-2-({methyl[(8S)-5,6,7,8-tetrahydro-8-quinolinyl]amino}methyl)imidazo[1,2-a]pyridine-3-carbaldehyde (100 mg, 0.24 mmol), hydroxylamine hydrochloride (21 mg, 0.30 mmol), and sodium formate (29 mg, 0.43 mmol) in formic acid (1 mL) was heated to reflux for 3 hours. The reaction was concentrated, dissolved in dichloromethane, and washed with saturated aqueous sodium carbonate. The organic layers were combined, dried with magnesium sulfate, filtered, concentrated, and purified by flash chromatography (0-10% ammonium hydroxide in acetonitrile) to give 63 mg (63% yield) of 5-(4-methyl-1-piperazinyl)-2-({methyl[(8S)-5,6,7,8-tetrahydro-8-quinolinyl]amino}methyl)imidazo[1,2-a]pyridine-3-carbonitrile as a tan solid. ¹H NMR (400 MHz, CDCl₃) δ 8.48 (d, J=4.2 Hz, 1H), 7.45 (d, J=8.8 Hz, 1H), 7.37-7.33 (m, 2H), 7.05 (dd, J=7.6, 4.7 Hz, 1H), 6.57 (d, J=7.3 Hz, 1H), 4.06 (m, 1H), 4.04 (s, 2H), 3.35-3.30 (m, 2H), 3.04-2.98 (m, 2H), 2.88-2.66 (m, 6H), 2.45 (s, 3H), 2.43 (s, 3H), 2.16-2.04 (m, 3H), 1.72 (m, 1H); MS m/z 416 (M+1).

### Example 18

### 5-(4-Methyl-1-piperazinyl)-2-({methyl[(8S)-5,6,7,8-tetrahydro-8-quinolinyl]amino}methyl)imidazo[1,2-a]pyridine-3-carboxamide

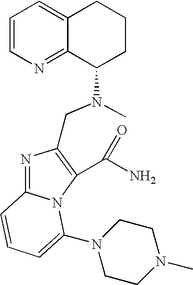

A solution of 5-(4-methyl-1-piperazinyl)-2-({methyl[(8S)-5,6,7,8-tetrahydro-8-quinolinyl]amino}methyl)imidazo[1,2-a]pyridine-3-carbonitrile (120 mg, 0.29 mmol) in concentrated sulfuric acid (1 mL) was heated at 90° C. for 1½ hours. The reaction mixture was cooled to room temperature, concentrated, diluted with water, basified with saturated aqueous sodium carbonate, extracted into dichloromethane/isopropanol, dried over magnesium sulfate, filtered, concentrated, and purified by flash chromatography (0-10% ammonium hydroxide in acetonitrile) to give 66 mg (53% yield) of 5-(4-methyl-1-piperazinyl)-2-({methyl[(8S)-5,6,7,8-tetrahydro-8-quinolinyl]amino}methyl)imidazo[1,2-a]pyridine-3-carboxamide as a white solid. ¹H NMR (400 MHz, CDCl₃) δ 10.24 (s, 1H), 8.34 (d, J=4.3 Hz, 1H), 7.30 (d, J=7.6 Hz, 1H), 7.24-7.21 (m, 2H), 7.01-6.98 (m, 1H), 6.33 (d, J=5.8 Hz, 1H), 5.77 (s, 1H), 4.11 (d, J=13.1 Hz, 1H), 4.00 (d, J=13.0 Hz, 1H), 3.94 (t, J=7.8 Hz, 1H), 3.46-3.31 (m, 2H), 2.80-2.58 (m, 7H), 2.38, 1.07 (t, J=7.1 Hz, (s, 3H), 2.13 (m, 1H), 2.08 (s, 3H), 2.01-1.92 (m, 3H), 1.67 (m, 1H); MS m/z 434 (M+1).

### Example 19

### [2-({Ethyl[(8S)-5,6,7,8-tetrahydro-8-quinolinyl]amino}methyl)-5-(4-methyl-1-piperazinyl)imidazo[1,2-a]pyridin-3-yl]methanol (Intermediate)

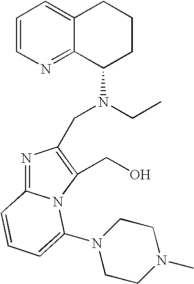

### A) 6-Fluoro-2-pyridinamine

A solution of 2,6-difluoropyridine (50 g, 434 mmol) in ammonium hydroxide (200 mL, 28.0-30.0%) was heated at 105° C. in a steel bomb for 15 hours. The reaction was cooled in an ice bath and the precipitate filtered, rinsed with cold water, and dried to yield 6-fluoro-2-pyridinamine (45.8 g, 94% yield) as a white solid. ¹H-NMR (CDCl₃): δ 7.53 (m, 1H), 6.36 (dd, 1H), 6.26 (dd, 1H), 4.56 (s, 2H).

### B) 2-(Dichloromethyl)-5-fluoroimidazo[1,2-a]pyridine

A solution of 6-fluoro-2-pyridinamine (67 g, 0.60 mol) in ethylene glycol dimethyl ether (570 mL) was treated with 1,1,3-trichloroacetone (190 mL, 1.80 mol) and heated at 85° C. for 15 hours. The reaction was cooled in an ice bath and the precipitate filtered, rinsed with hexanes, and dried to yield 2-(dichloromethyl)-5-fluoroimidazo[1,2-a]pyridine (85 g, 65% yield) as an olive green solid. ¹H-NMR (CDCl₃): δ 8.18 (s, 1H), 7.60 (s, 1H), 7.54-7.46 (m, 2H), 6.93 (m, 1H).

### C) 5-Fluoroimidazo[1,2-a]pyridine-2-carbaldehyde

A solution of 2-(dichloromethyl)-5-fluoroimidazo[1,2-a]pyridine (103 g, 470 mmol) in ethanol (300 mL) and water (600 mL) was treated with sodium acetate (96 g, 1.17 mol) and heated at 60° C. for 2 hours. The reaction was cooled, filtered though celite, and concentrated in vacuo to remove the ethanol. The aqueous was extracted twice with chloroform and the organics were combined, washed with water and brine, dried over sodium sulfate, and concentrated. The residue was filtered through a pad of silica, rinsed with dichloromethane and ethyl acetate, concentrated, triturated with hexanes, filtered, and dried to yield 5-fluoroimidazo[1,2-a]pyridine-2-carbaldehyde (40 g, 52% yield) as a tan solid. ¹H-NMR (CDCl₃): δ 10.17 (s, 1H), 8.22 (s, 1H), 7.57 (d, 1H), 7.38-7.32 (m, 1H), 6.60 (m, 1H); TLC (10% 2 M ammonia in methy alcohol-ethyl acetate) R_{f}=0.60.

### D) (5-Fluoroimidazo[1,2-a]pyridin-2-yl)methanol

A solution of 5-fluoroimidazo[1,2-a]pyridine-2-carbaldehyde (80 g, 490 mmol) in methanol (1 L) at 0° C. was treated with sodium borohydride (24 g, 640 mmol) in portions. The reaction was slowly brought to room temperature, stirred for 2 hours, quenched with water, concentrated, dissolved in 3:1 dichloromethane to isopropyl alcohol, and washed with saturated aqueous sodium bicarbonate. The organic layer was separated and the aqueous extracted four times with 3:1 dichloromethane to isopropyl alcohol. The organic layers were combined, dried over sodium sulfate, concentrated, triturated with hexanes, and filtered to yield (5-fluoroimidazo[1,2-a]pyridin-2-yl)methanol (76 g, 93% yield) as a brown solid. ¹H-NMR (CDCl₃): δ 7.59 (s, 1H), 7.38 (d, 1H), 7.21-7.15 (m, 1H), 6.43 (m, 1H), 4.85 (s, 2H), 4.45 (s, 1H).

### E) [5-(4-Methyl-1-piperazinyl)imidazo[1,2-a]pyridin-2-yl]methanol

A solution of (5-fluoroimidazo[1,2-a]pyridin-2-yl)methanol (76 g, 460 mmol) in 1-methyl piperazine (150 mL) was heated at 70° C. for 15 hours. The reaction mixture was cooled, poured into 1.3 L brine, and extracted into 3:1 chloroform to isopropyl alcohol. The combined extracts were dried over sodium sulfate, concentrated in vacuo, azeotroped with hexanes, and triturated with diethyl ether to yield [5-(4-methyl-1-piperazinyl)imidazo[1,2-a]pyridin-2-yl]methanol (101 g, 90% yield) as a tan solid. ¹H-NMR (CDCl₃): δ 7.51 (s, 1H), 7.33 (d, 1H), 7.21-7.17 (m, 1H), 6.31 (m, 1H), 4.87 (s, 2H), 3.17 (s, 4H), 2.68 (s, 4H), 2.42 (s, 3H).

### F) 5-(4-Methyl-1-piperazinyl)imidazo[1,2-a]pyridine-2-carbaldehyde

A solution of [5-(4-methyl-1-piperazinyl)imidazo[1,2-a]pyridin-2-yl]methanol (101 g, 410 mmol) in chloroform (1650 mL) was treated with manganese dioxide (360 g, 4100 mmol) and stirred at room temperature for 72 hours. The reaction mixture was filtered through celite, rinsed with chloroform, and concentrated to yield 5-(4-methyl-1-piperazinyl)imidazo[1,2-a]pyridine-2-carbaldehyde (82 g, 82% yield) as gold solid. ¹H-NMR (CDCl₃): δ 10.17 (s, 1H), 8.15 (s, 1H), 7.44 (d, 1H), 7.31-7.27 (m, 1H), 6.40 (m, 1H), 3.16 (s, 4H), 2.68 (s, 4H), 2.42 (s, 3H).

### G) (8S)—N-{(1S)-1-[4-(Methyloxy)phenyl]ethyl}-5,6,7,8-tetrahydro-8-quinolinamine

A solution of (S)-(−)-1-(4-methoxyphenyl)ethylamine (25 g, 166 mmol) and 6,7-dihydro-8(5H)-quinolinone (24 g, 166 mmol) in dichloromethane was treated with glacial acetic acid (14 mL, 249 mmol) and sodium triacetoxyborohydride (53 g, 249 mmol). The reaction mixture was stirred at room temperature for 15 hours and treated with sodium carbonate (106 g, 996 mmol) and stirred for 30 minutes. The mixture was diluted with dichloromethane, the organic layer separated, and the aqueous extracted with more dichloromethane. The organic layers were combined, dried over magnesium sulfate, concentrated, and purified by column chromatography (0-3% 2 M ammonia in methanol/dichloromethane) to give a yellow oil which was crystallized from hexanes to yield (8S)—N-{(1S)-1-[4-(methyloxy)phenyl]ethyl}-5,6,7,8-tetrahydro-8-quinolinamine (33 g, 70% yield) as clear crystals. ¹H-NMR (CDCl₃): δ 8.40 (m, 1H), 7.33 (m, 3H), 7.04 (m, 1H), 6.84 (d, 2H), 4.02 (m, 1H), 3.83-3.78 (m, 4H), 2.73-2.62 (m, 2H), 1.82 (m, 1H), 1.72 (m, 1H), 1.57 (m, 2H), 1.43 (d, 3H).

### H) (8S)—N-{(1S)-1-[4-(Methyloxy)phenyl]ethyl}-N-{[5-(4-methyl-1-piperazinyl)imidazo[1,2-a]pyridin-2-yl]methyl}-5,6,7,8-tetrahydro-8-quinolinamine

A solution of 5-(4-methyl-1-piperazinyl)imidazo[1,2-a]pyridine-2-carbaldehyde (2.83 g, 11.6 mmol) and (8S)—N-{(1S)-1-[4-(methyloxy)phenyl]ethyl}-5,6,7,8-tetrahydro-8-quinolinamine (3.27 g, 11.6 mmol) in dichloroethane (40 mL) was treated with glacial acetic acid (1.0 mL, 17.4 mmol) and sodium triacetoxyborohydride (3.68 g, 17.4 mmol, added in portions) and stirred at room temperature for 15 hours. The reaction mixture was diluted with dichloromethane, washed with saturated aqueous sodium bicarbonate, separated, and extracted with additional dichloromethane. The organic layers were combined, washed with brine, dried over sodium sulfate, concentrated, and purified by flash chromatography (0-4% ammonium hydroxide in acetonitrile). The residue was dissolved in dichloromethane and stirred with 2 M ammonia in methanol to yield (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine (5.13 g, 87% yield) as pale yellow foam. ¹H NMR (400 MHz, CDCl₃) δ 8.48 (d, J=4.6 Hz, 1H), 7.78 (s, 1H), 7.60-7.58 (m, 2H), 7.24-7.18 (m, 2H), 7.09-7.05 (m, 1H), 6.97 (dd, J=7.6, 4.7 Hz, 1H), 6.84-6.82 (m, 2H), 6.21 (d, J=7.2 Hz, 1H), 4.82 (m, 1H), 4.07 (m, 1H), 3.91 (dd, J=56.9, 17.1 Hz, 2H), 3.77 (s, 3H), 3.19-3.13 (m, 4 H), 2.74 (s, 4H), 2.67-2.53 (m, 2H), 2.47 (s, 3H), 2.06 (m, 1H), 1.85 (m, 2H), 1.53 (m, 1H), 1.34 (d, J=6.4 Hz, 3H); MS m/z 511 (M+1).

### I): [2-({{(1S)-1-[4-(Methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol

[2-({{(1S)-1-[4-(Methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine via hydroxymethylation in a similar manner as shown herein to give an off-white solid (80% yield). ¹H NMR (400 MHz, CDCl₃) δ 8.42 (d, J=4.2 Hz, 1H), 7.43 (d, J=8.6 Hz, 2H), 7.28-7.21 (m, 2H), 7.05-7.01 (m, 1H), 6.95 (dd, J=7.6, 4.8 Hz, 1H), 6.82-6.80 (m, 2H), 6.38 (d, J=7.1 Hz, 1H), 5.19 (d, J=12.9 Hz, 1H), 4.61 (d, J=12.9 Hz, 1H), 4.09-4.06 (m, 2H), 4.03-3.93 (m, 2H), 3.77 (s, 3H), 3.63 (m, 2H), 3.08 (m, 1H), 2.92-2.80 (m, 4H), 2.71 (m, 1H), 2.59-2.52 (m, 2H), 2.47-2.44 (m, 2H), 2.40 (s, 3H), 2.32 (m, 1H), 2.16 (m, 1H), 1.96 (m, 1H), 1.55 (d, J=7.1 Hz, 3H); MS m/z 541 (M+1).

### J) [2-({Ethyl[(8S)-5,6,7,8-tetrahydro-8-quinolinyl]amino}methyl)-5-(4-methyl-1-piperazinyl)imidazo[1,2-a]pyridin-3-yl]methanol

A solution of [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol (166 mg, 0.31 mmol) in dichloromethane (1 mL) was treated with trifluoroacetic acid (0.5 mL) and stirred at room temperature for 1.25 hours. The reaction was concentrated, diluted with dichloromethane, and washed with saturated aqueous sodium carbonate. The organic layer was separated, concentrated, and purified by flash chromatography (0-10% ammonium hydroxide in acetonitrile) to give 31 mg (25% yield) of the deprotected intermediate (5-(4-methyl-1-piperazinyl)-2-{[(8S)-5,6,7,8-tetrahydro-8-quinolinylamino]methyl}imidazo[1,2-a]pyridin-3-yl)methanol. This intermediate (30 mg, 0.074 mmol) was dissolved in dichloroethane (750 μL) and treated with acetaldehyde (8.3 μL, 0.15 mmol), glacial acetic acid (6.3 μL, 0.11 mmol), and sodium triacetoxyborohydride (24 mg, 0.11 mmol) and stirred at room temperature for 15 hours. The reaction was diluted with dichloromethane and washed with saturated aqueous sodium carbonate. The organic layer was separated and the aqueous extracted with dichloromethane. The organic layers were combined, concentrated, and purified by preparative chromatography (0-10% ammonium hydroxide in acetonitrile) to give 22 mg (69% yield) [2-({ethyl[(8S)-5,6,7,8-tetrahydro-8-quinolinyl]amino}methyl)-5-(4-methyl-1-piperazinyl)imidazo[1,2-a]pyridin-3-yl]methanol as a white solid. ¹H NMR (400 MHz, CDCl₃) δ 8.36 (d, J=4.4 Hz, 1H), 7.31-7.26 (m, 2H), 7.06 (m, 1H), 6.98 (dd, J=7.5, 4.9 Hz, 1H), 6.40 (d, J=7.2 Hz, 1H), 5.52 (d, J=13.2 Hz, 1H), 5.20 (d, J=12.9 Hz, 1H), 4.19 (d, J=14.0 Hz, 1H), 3.89-3.85 (m, 2H), 3.75 (m, 1H), 3.18 (m, 1H), 3.00-2.88 (m, 3H), 2.80-2.71 (m, 2H), 2.65-2.56 (m, 2H), 2.51-2.45 (m, 2H), 2.40 (s, 3H), 2.38-2.33 (m, 2H), 1.97 (m, 1H), 1.79 (m, 1H), 1.59 (m, 1H), 1.13 (t, J=7.1 Hz, 3H); MS m/z 435 (M+1).

Alternatively, [2-({Ethyl[(8S)-5,6,7,8-tetrahydro-8-quinolinyl]amino}methyl)-5-(4-methyl-1-piperazinyl)imidazo[1,2-a]pyridin-3-yl]methanol may be made from (8S)—N-Ethyl-N-{[5-(4-methyl-1-piperazinyl)imidazo[1,2-a]pyridin-2-yl]methyl}-5,6,7,8-tetrahydro-8-quinolinamine by hydroxymethylation.

### Example 20

### 2-({Ethyl[(8S)-5,6,7,8-tetrahydro-8-quinolinyl]amino}methyl)-5-(4-methyl-1-piperazinyl)imidazo[1,2-a]pyridine-3-carbaldehyde (Intermediate)

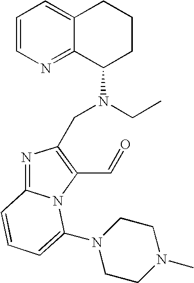

A solution of [2-({ethyl[(8S)-5,6,7,8-tetrahydro-8-quinolinyl]amino}methyl)-5-(4-methyl-1-piperazinyl)imidazo[1,2-a]pyridin-3-yl]methanol (108 mg, 0.25 mmol) in N,N-dimethylformamide (2 mL) was treated with IBX polystyrene (341 mg, 0.38 mmol) and stirred at room temperature for 15 hours. The reaction mixture was filtered, rinsed with dichloromethane, dissolved in methanol, heated at 40° C. for 15 hours, filtered, concentrated, and purified by flash chromatography (0-10% ammonium hydroxide in acetonitrile) to give 37 mg (34% yield) of 2-({ethyl[(8S)-5,6,7,8-tetrahydro-8-quinolinyl]amino}methyl)-5-(4-methyl-1-piperazinyl)imidazo[1,2-a]pyridine-3-carbaldehyde as a reddish tan solid. ¹H NMR (400 MHz, CDCl₃) δ 10.83 (s, 1H), 8.41 (d, J=4.3 Hz, 1H), 7.44 (d, J=8.6 Hz, 1H), 7.34 (d, J=7.8 Hz, 1H), 7.28 (m, 1H), 6.96 (dd, J=7.5, 4.8 Hz, 1H), 6.56 (d, J=7.4 Hz, 1H), 4.33 (m, 1H), 4.30 (d, J=15.0 Hz, 1H), 4.19 (d, J=15.8 Hz, 1H), 3.34-3.26 (m, 2H), 2.92-2.76 (m, 6H), 2.67-2.60 (m, 2H), 2.40-2.37 (m, 2H), 2.35 (s, 3H), 2.13 (m, 1H), 2.02-1.92 (m, 2H), 1.64 (m, 1H), 1.06 (t, J=7.1 Hz, 3H); MS m/z 433 (M+1).

### Example 21

### 2-({Ethyl[(8S)-5,6,7,8-tetrahydro-8-quinolinyl]amino}methyl)-5-(4-methyl-1-piperazinyl)imidazo[1,2-a]pyridine-3-carbonitrile

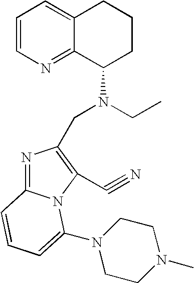

2-({Ethyl[(8S)-5,6,7,8-tetrahydro-8-quinolinyl]amino}methyl)-5-(4-methyl-1-piperazinyl)imidazo[1,2-a]pyridine-3-carbonitrile was prepared from 2-({ethyl[(8S)-5,6,7,8-tetrahydro-8-quinolinyl]amino}methyl)-5-(4-methyl-1-piperazinyl)imidazo[1,2-a]pyridine-3-carbaldehyde and hydroxylamine hydrochloride in a similar manner as described herein to give a tan solid (71% yield). ¹H NMR (400 MHz, CDCl₃) δ 8.43 (d, J=4.5 Hz, 1H), 7.42 (d, J=8.9 Hz, 1H), 7.35-7.29 (m, 2H), 6.99 (dd, J=7.6, 4.8 Hz, 1H), 6.54 (d, J=7.2 Hz, 1H), 4.19 (m, 1H), 4.13 (d, J=14.6 Hz, 1H), 3.95 (d, J=14.5 Hz, 1H), 3.32-3.29 (m, 2H), 3.01-2.88 (m, 3H), 2.84-2.61 (m, 7H), 2.39 (s, 3H), 2.15 (m, 1), 2.05-1.91 (m, 2H), 1.67 (m, 1H), 1.09-1.06 (m, 3H); MS m/z 430 (M+1).

### Example 22

### 2-({Ethyl[(8S)-5,6,7,8-tetrahydro-8-quinolinyl]amino}methyl)-5-(4-methyl-1-piperazinyl)imidazo[1,2-a]pyridine-3-carboxamide

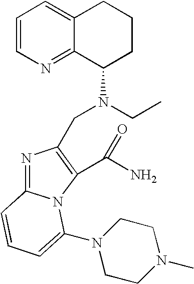

2-({Ethyl[(8S)-5,6,7,8-tetrahydro-8-quinolinyl]amino}methyl)-5-(4-methyl-1-piperazinyl)imidazo[1,2-a]pyridine-3-carboxamide was prepared from 2-({ethyl[(8S)-5,6,7,8-tetrahydro-8-quinolinyl]amino}methyl)-5-(4-methyl-1-piperazinyl)imidazo[1,2-a]pyridine-3-carbonitrile and sulfuric acid in a similar manner as described herein to give an off-white solid (53% yield). ¹H NMR (400 MHz, CDCl₃) δ 10.48 (s, 1H), 8.31 (d, J=4.4 Hz, 1H), 7.29-7.20 (m, 3H), 6.98 (dd, J=7.6, 4.7 Hz, 1H), 6.34 (d, J=6.6 Hz, 1H), 5.71 (s, 1H), 4.22 (d, J=12.7 Hz, 1H), 3.99 (d, J=12.8 Hz, 1H), 3.88 (m, 1H), 3.44-3.31 (m, 2H), 2.81-2.52 (m, 8H), 2.41 (s, 3H), 2.26-2.19 (m, 2H), 2.03-1.89 (m, 3H), 1.63 (m, 1H), 0.93 (m, 3H); MS m/z 448 (M+1).

### Example 23

### [5-(4-Methyl-1-piperazinyl)-2-({propyl[(8S)-5,6,7,8-tetrahydro-8-quinolinyl]amino}methyl)imidazo[1,2-a]pyridin-3-yl]methanol (Intermediate)

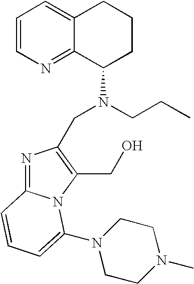

[5-(4-Methyl-1-piperazinyl)-2-({propyl[(8S)-5,6,7,8-tetrahydro-8-quinolinyl]amino}methyl)imidazo[1,2-a]pyridin-3-yl]methanol was prepared from [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol and propionaldehyde via deprotection and reductive amination in a similar manner as shown herein to give a white solid (64% yield). ¹H NMR (400 MHz, CDCl₃) δ 8.36 (d, J=4.5 Hz, 1H), 7.32-7.26 (m, 2H), 7.06 (dd, J=8.7, 7.2 Hz, 1H), 6.98 (dd, J=7.7, 4.7 Hz, 1H), 6.41 (d, J=7.1 Hz, 1H), 5.52 (d, J=12.9 Hz, 1H), 5.17 (d, J=13.0 Hz, 1H), 4.20 (d, J=13.4 Hz, 1H), 3.90 (d, J=13.5 Hz, 1H), 3.85 (m, 1H), 3.77 (m, 1H), 3.17 (m, 1H), 2.98 (m, 1H), 2.91 (m, 2H), 2.77 (m, 2H), 2.62 (m, 2H), 2.49 (m, 1H), 2.41 (s, 3H), 2.35 (m, 2H), 2.20 (m, 1H), 1.98 (m, 1H), 1.81 (m, 1H), 1.64-1.56 (m, 3H), 0.78 (t, J=7.3 Hz, 3H); MS m/z 449 (M+1).

Alternatively, [5-(4-Methyl-1-piperazinyl)-2-({propyl[(8S)-5,6,7,8-tetrahydro-8-quinolinyl]amino}methyl)imidazo[1,2-a]pyridin-3-yl]methanol may be formed from (8S)—N-{[5-(4-Methyl-1-piperazinyl)imidazo[1,2-a]pyridin-2-yl]methyl}-N-propyl-5,6,7,8-tetrahydro-8-quinolinamine by hydroxymethylation.

### Example 24

### 5-(4-Methyl-1-piperazinyl)-2-({propyl[(8S)-5,6,7,8-tetrahydro-8-quinolinyl]amino}methyl)imidazo[1,2-a]pyridine-3-carbaldehyde (Intermediate)

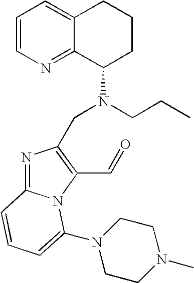

5-(4-Methyl-1-piperazinyl)-2-({propyl[(8S)-5,6,7,8-tetrahydro-8-quinolinyl]amino}methyl)imidazo[1,2-a]pyridine-3-carbaldehyde was prepared from [5-(4-methyl-1-piperazinyl)-2-({propyl[(8S)-5,6,7,8-tetrahydro-8-quinolinyl]amino}methyl)imidazo[1,2-a]pyridin-3-yl]methanol and IBX polystyrene in a similar manner as described herein to give a reddish tan solid (35% yield). ¹H NMR (400 MHz, CDCl₃) δ 10.83 (s, 1H), 8.41 (d, J=4.2 Hz, 1H), 7.43 (d, J=8.7 Hz, 1H), 7.35-7.27 (m, 2H), 6.97 (dd, J=7.5, 4.7 Hz, 1H), 6.56 (d, J=7.3 Hz, 1H), 4.38-4.20 (m, 3H), 3.35-3.27 (m, 2H), 2.90-2.60 (m, 8H), 2.47-2.39 (m, 2H), 2.35 (s, 3H), 2.13 (m, 1H), 2.03-1.91 (m, 2H), 1.65 (m, 1H), 1.52-1.42 (m, 2H), 0.74 (t, J=7.3 Hz, 3H); MS m/z 448 (M+1).

### Example 25

### 5-(4-Methyl-1-piperazinyl)-2-({propyl[(8S)-5,6,7,8-tetrahydro-8-quinolinyl]amino}methyl)imidazo[1,2-a]pyridine-3-carbonitrile

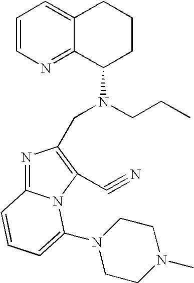

5-(4-Methyl-1-piperazinyl)-2-({propyl[(8S)-5,6,7,8-tetrahydro-8-quinolinyl]amino}methyl)imidazo[1,2-a]pyridine-3-carbonitrile was prepared from 5-(4-methyl-1-piperazinyl)-2-({propyl[(8S)-5,6,7,8-tetrahydro-8-quinolinyl]amino}methyl)imidazo[1,2-a]pyridine-3-carbaldehyde and hydroxylamine hydrochloride in a similar manner as described herein to give a tan solid (66% yield). ¹H NMR (400 MHz, CDCl₃) δ 8.42 (d, J=4.3 Hz, 1H), 7.43 (d, J=8.6 Hz, 1H), 7.35-7.29 (m, 2H), 6.99 (dd, J=7.5, 4.8 Hz, 1H), 6.55 (d, J=7.1 Hz, 1H), 4.24 (d, J=14.4 Hz, 1H), 4.17 (m, 1H), 4.00 (d, J=15.2 Hz, 1H), 3.31 (d, J=11.4 Hz, 2H), 3.03-2.95 (m, 2H), 2.85-2.74 (m, 6H), 2.68-2.54 (m, 2H), 2.41 (s, 3H), 2.17 (m, 1H), 2.04-1.91 (m, 2H), 1.66 (m, 1H), 1.52-1.43 (m, 2H), 0.77 (t, J=7.2 Hz, 3H); MS m/z 444 (M+1).

### Example 26

### 5-(4-Methyl-1-piperazinyl)-2-({propyl[(8S)-5,6,7,8-tetrahydro-8-quinolinyl]amino}methyl)imidazo[1,2-a]pyridine-3-carboxamide

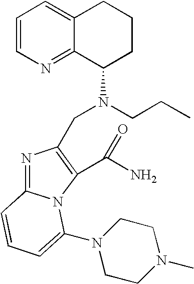

5-(4-Methyl-1-piperazinyl)-2-({propyl[(8S)-5,6,7,8-tetrahydro-8-quinolinyl]amino}methyl)imidazo[1,2-a]pyridine-3-carboxamide was prepared from 5-(4-methyl-1-piperazinyl)-2-({propyl[(8S)-5,6,7,8-tetrahydro-8-quinolinyl]amino}methyl)imidazo[1,2-a]pyridine-3-carbonitrile and sulfuric acid in a similar manner as described herein to give an off-white solid (63% yield). ¹H NMR (400 MHz, CDCl₃) δ 10.42 (s, 1H), 8.31 (d, J=4.4 Hz, 1H), 7.29-7.22 (m, 3H), 6.98 (dd, J=7.6, 4.6 Hz, 1H), 6.34 (dd, J=6.5, 1.7 Hz, 1H), 5.69 (s, 1H), 4.20 (d, J=12.9 Hz, 1H), 4.01 (d, J=13.6 Hz, 1H), 3.88 (m, 1H), 3.43-3.30 (m, 2H), 2.82-2.57 (m, 7H), 2.50-2.43 (m, 2H), 2.41 (s, 3H), 2.21 (m, 1H), 2.02-1.90 (m, 3H), 1.63 (m, 1H), 1.44-1.24 (m, 2H), 0.69 (t, J=7.3 Hz, 3H); MS m/z 462 (M+1).

### Example 27

### [2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]ethanol (Intermediate)

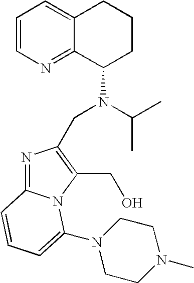

[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol was prepared from [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol and acetone via deprotection and reductive amination in a similar manner as shown herein to give a white solid (39% yield). ¹H NMR (400 MHz, CDCl₃) δ 8.31 (s, 1H), 7.31-7.25 (m, 2 H), 7.05 (m, 1H), 6.96 (m, 1H), 6.40 (d, J=7.1 Hz, 1H), 5.55 (d, J=13.0 Hz, 1H), 5.14-5.11 (m, 1H), 4.20-4.16 (m, 1H), 4.00-3.97 (m, 1H), 3.83-3.79 (m, 2H), 3.12 (m, 1H), 3.01 (m, 1H), 2.93-2.88 (m, 2H), 2.81-2.70 (m, 3H), 2.65-2.58 (m, 2H), 2.51-2.44 (m, 2H), 2.41 (s, 3H), 1.97 (m, 1H), 1.82 (m, 1H), 1.57 (m, 1H), 1.19 (m, 6H); MS m/z 449 (M+1).

Alternatively, [2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol may be formed from (8S)—N-(1-Methylethyl)-N-{[5-(4-methyl-1-piperazinyl)imidazo[1,2-a]pyridin-2-yl]methyl}-5,6,7,8-tetrahydro-8-quinolinamine by hydroxymethylation.

### Example 28

### 2-({(1-Methylethyl)[(8S)-5,6,7,8-tetrahydro-8-quinolinyl]amino}methyl)-5-(4-methyl-1-piperazinyl)imidazo[1,2-a]pyridine-3-carbaldehyde (Intermediate)

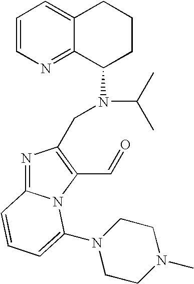

2-({(1-Methylethyl)[(8S)-5,6,7,8-tetrahydro-8-quinolinyl]amino}methyl)-5-(4-methyl-1-piperazinyl)imidazo[1,2-a]pyridine-3-carbaldehyde was prepared from [2-({(1-methylethyl)[(8S)-5,6,7,8-tetrahydro-8-quinolinyl]amino}methyl)-5-(4-methyl-1-piperazinyl)imidazo[1,2-a]pyridin-3-yl]methanol and IBX polystyrene in a similar manner as described herein to give a reddish tan solid (24% yield). ¹H NMR (400 MHz, CDCl₃) δ 10.80 (s, 1H), 8.37 (d, J=4.4 Hz, 1H), 7.42 (d, J=8.6 Hz, 1H), 7.32 (t, J=8.0 Hz, 1H), 7.20 (d, J=7.6 Hz, 1H), 6.91 (dd, J=7.4, 4.7 Hz, 1H), 6.53 (d, J=7.2 Hz, 1H), 4.31 (m, 1H), 4.24-4.21 (m, 2H), 3.31-3.24 (m, 3H), 2.92-2.75 (m, 6 H), 2.43-2.38 (m, 2H), 2.36 (s, 3H), 2.10-1.97 (m, 3H), 1.62 (m, 1H), 1.13 (m, 6H); MS m/z 447 (M+1).

### Example 29

### 2-({(1-Methylethyl)[(8S)-5,6,7,8-tetrahydro-8-quinolinyl]amino}methyl)-5-(4-methyl-1-piperazinyl)imidazo[1,2-a]pyridine-3-carbonitrile

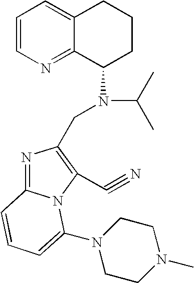

2-({(1-Methylethyl)[(8S)-5,6,7,8-tetrahydro-8-quinolinyl]amino}methyl)-5-(4-methyl-1-piperazinyl)imidazo[1,2-a]pyridine-3-carbonitrile was prepared from 2-({(1-methylethyl)[(8S)-5,6,7,8-tetrahydro-8-quinolinyl]amino}methyl)-5-(4-methyl-1-piperazinyl)imidazo[1,2-a]pyridine-3-carbaldehyde and hydroxylamine hydrochloride in a similar manner as described herein to give a green solid (67% yield). ¹H NMR (400 MHz, CDCl₃) δ 8.39 (m, 1H), 7.38 (d, J=8.8 Hz, 1H), 7.31 (m, 1H), 7.23 (m, 1H), 6.93 (m, 1H), 6.52 (d, J=7.3 Hz, 1H), 4.20 (m, 1H), 4.13 (d, J=15.7 Hz, 1H), 3.92 (d, J=15.6 Hz, 1H), 3.31-3.23 (m, 3H), 3.02-2.92 (m, 2H), 2.83-2.74 (m, 4H), 2.66-2.58 (m, 2H), 2.41 (s, 3H), 2.11-2.02 (m, 3H), 1.67 (m, 1H), 1.15 (d, J=6.5 Hz, 6H); MS m/z 444 (M+1).

### Example 30

### 2-([(1-Methylethyl)[(8S)-5,6,7,8-tetrahydro-8-quinolinyl]amino]methyl)-5-(4-methyl-1-piperazinyl)imidazo[1,2-a]pyridine-3-carboxamide

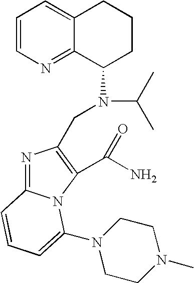

2-({(1-Methylethyl)[(8S)-5,6,7,8-tetrahydro-8-quinolinyl]amino}methyl)-5-(4-methyl-1-piperazinyl)imidazo[1,2-a]pyridine-3-carboxamide was prepared from 2-({(1-methylethyl)[(8S)-5,6,7,8-tetrahydro-8-quinolinyl]amino}methyl)-5-(4-methyl-1-piperazinyl)imidazo[1,2-a]pyridine-3-carbonitrile and sulfuric acid in a similar manner as described herein to give a pale yellow solid (50% yield). ¹H NMR (400 MHz, CDCl₃) δ 10.53 (s, 1H), 8.25 (d, J=4.3 Hz, 1H), 7.24-7.17 (m, 3H), 6.93 (dd, J=7.5, 4.7 Hz, 1H), 6.32 (d, J=6.9 Hz, 1H), 5.71 (s, 1H), 4.27 (d, J=13.0 Hz, 1H), 4.05 (d, J=13.0 Hz, 1H), 3.84 (m, 1H), 3.36-3.23 (m, 2H), 2.97-2.56 (m, 8H), 2.41 (s, 3H), 2.33 (m, 1H), 2.05-1.98 (m, 2H), 1.83 (m, 1H), 1.59 (m, 1H), 1.20 (d, J=6.4 Hz, 3H), 1.02 (d, J=6.3 Hz, 3H); MS m/z 462 (M+1).

### Example 31

### [2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol (Intermediate)

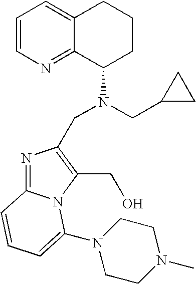

[2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol was prepared from [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol and cyclopropane carboxaldehyde via deprotection and reductive amination in a similar manner as shown herein to give a white solid (69% yield). ¹H NMR (400 MHz, CDCl₃) δ 8.37 (d, J=4.6 Hz, 1H), 7.33-7.26 (m, 2H), 7.07 (dd, J=8.7, 7.3 Hz, 1H), 6.99 (dd, J=7.5, 4.7 Hz, 1H), 6.42 (d, J=6.9 Hz, 1H), 5.54 (d, J=13.0 Hz, 1H), 5.31 (d, J=13.1 Hz, 1H), 4.47 (d, J=14.0 Hz, 1H), 3.92-3.89 (m, 2H), 3.78 (m, 1H), 3.18 (m, 1H), 3.01-2.89 (m, 3H), 2.83-2.71 (m, 2H), 2.64-2.58 (m, 2H), 2.50 (m, 1H), 2.42 (s, 3H), 2.36-2.29 (m, 2H), 2.16 (m, 1H), 1.99-1.86 (m, 2H), 1.76 (m, 1H), 1.59 (m, 1H), 1.06 (m, 1H), 0.58 (m, 1H), 0.39 (m, 1H), 0.11 (m, 1H), 0.03 (m, 1H); MS m/z 461 (M+1).

Alternatively, [2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol may be formed from (8S)—N-(Cyclopropylmethyl)-N-{[5-(4-methyl-1-piperazinyl)imidazo[1,2-a]pyridin-2-yl]methyl}-5,6,7,8-tetrahydro-8-quinolinamine by hydroxymethylation.

### Example 32

### 2-({(Cyclopropylmethyl)[(8S)-5,6,7,8-tetrahydro-8-quinolinyl]amino}methyl)-5-(4-methyl-1-piperazinyl)imidazo[1,2-a]pyridine-3-carbaldehyde (Intermediate)

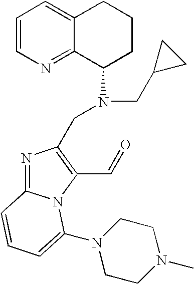

2-({(Cyclopropylmethyl)[(8S)-5,6,7,8-tetrahydro-8-quinolinyl]amino}methyl)-5-(4-methyl-1-piperazinyl)imidazo[1,2-a]pyridine-3-carbaldehyde was prepared from [2-({(cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol and IBX polystyrene in a similar manner as described herein to give a reddish tan solid (37% yield). ¹H NMR (400 MHz, CDCl₃) δ 10.84 (s, 1H), 8.41 (d, J=4.4 Hz, 1H), 7.43 (d, J=8.6 Hz, 1H), 7.35-7.31 (m, 1H), 7.28-7.26 (m, 1H), 6.96 (dd, J=7.5, 4.6 Hz, 1H), 6.56 (d, J=7.3 Hz, 1H), 4.45 (m, 1H), 4.38 (d, J=15.4 Hz, 1H), 4.26 (d, J=15.5 Hz, 1H), 3.33-3.27 (m, 2H), 2.90-2.55 (m, 8H), 2.41-2.37 (m, 2H), 2.35 (s, 3H), 2.14 (m, 1H), 2.01-1.91 (m, 2H), 1.65 (m, 1H), 0.95 (m, 1H), 0.34 (d, J=8.4 Hz, 2H), 0.08-0.02 (m, 2H); MS m/z 459 (M+1).

### Example 33

### 2-({(Cyclopropylmethyl)[(8S)-5,6,7,8-tetrahydro-8-quinolinyl]amino}methyl)-5-(4-methyl-1-piperazinyl)imidazo[1,2-a]pyridine-3-carbonitrile

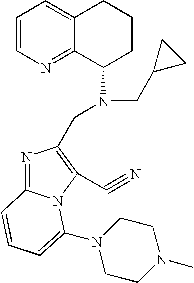

2-({(Cyclopropylmethyl)[(8S)-5,6,7,8-tetrahydro-8-quinolinyl]amino}methyl)-5-(4-methyl-1-piperazinyl)imidazo[1,2-a]pyridine-3-carbonitrile was prepared from 2-({(cyclopropylmethyl)[(8S)-5,6,7,8-tetrahydro-8-quinolinyl]amino}methyl)-5-(4-methyl-1-piperazinyl)imidazo[1,2-a]pyridine-3-carbaldehyde and hydroxylamine hydrochloride in a similar manner as described herein to give a tan solid (73% yield). ¹H NMR (400 MHz, CDCl₃) δ 8.42 (d, J=4.2 Hz, 1H), 7.42 (d, J=8.7 Hz, 1H), 7.35-7.28 (m, 2H), 6.98 (dd, J=7.6, 4.8 Hz, 1H), 6.55 (d, J=7.1 Hz, 1H), 4.36 (m, 1H), 4.24 (d, J=15.0 Hz, 1H), 4.06 (d, J=14.5 Hz, 1H), 3.31 (d, J=11.0 Hz, 2H), 3.01-2.92 (m, 2H), 2.83-2.71 (m, 6H), 2.67-2.58 (m, 2H), 2.39 (s, 3H), 2.20 (m, 1H), 2.03-1.91 (m, 2H), 1.67 (m, 1H), 0.96 (m, 1H), 0.41-0.32 (m, 2H), 0.10 (m, 1H), 0.01 (m, 1H); MS m/z 456 (M+1).

### Example 34

### (8S)—N-[(5-Fluoroimidazo[1,2-a]pyridin-2-yl)methyl]-N-{(1S)-1-[4-(methyloxy)phenyl]ethyl}-5,6,7,8-tetrahydro-8-quinolinamine (Intermediate)

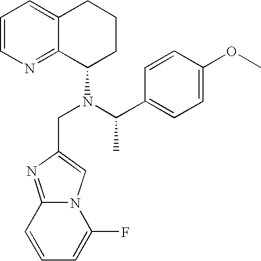

A solution of 5-fluoroimidazo[1,2-a]pyridine-2-carbaldehyde (2.5 g, 15.2 mmol) and (8S)—N-{(1S)-1-[4-(methyloxy)phenyl]ethyl}-5,6,7,8-tetrahydro-8-quinolinamine (4.3 g, 15.2 mmol) in dichloroethane (75 mL) was treated with glacial acetic acid (1.31 mL, 22.8 mmol) and sodium triacetoxyborohydride (4.83 g, 22.8 mmol, added in portions) and stirred at room temperature for 15 hours. The reaction mixture was diluted with dichloromethane, washed with saturated aqueous sodium carbonate, separated, and extracted with additional dichloromethane. The organic layers were combined, dried over magnesium sulfate, concentrated, and purified by flash chromatography (0-5% ammonium hydroxide in acetonitrile) to yield (8S)—N-[(5-fluoroimidazo[1,2-a]pyridin-2-yl)methyl]-N-{(1S)-1-[4-(methyloxy)phenyl]ethyl}-5,6,7,8-tetrahydro-8-quinolinamine (5.64 g, 86% yield) as a tan solid. ¹H NMR (400 MHz, CDCl₃) δ 8.48 (d, J=4.6 Hz, 1H), 7.81 (s, 1H), 7.55-7.53 (m, 2H), 7.28 (d, J=8.9 Hz, 1H), 7.21 (d, J=7.5 Hz, 1H), 7.13-7.07 (m, 1H), 6.97 (dd, J=7.6, 4.7 Hz, 1H), 6.88-6.86 (m, 2H), 6.39 (dd, J=7.3, 5.1 Hz, 1H), 4.97 (q, J=6.7 Hz, 1H), 4.04 (m, 1H), 4.01-3.97 (m, 2H), 3.79 (s, 3H), 2.69-2.53 (m, 2H), 2.07 (m, 1H), 1.88-1.73 (m, 2H), 1.52 (m, 1H), 1.29 (d, J=6.4 Hz, 3H); MS m/z 431 (M+1).

### Example 35

### (8S)—N-({5-[(3R)-3-(Dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine (Intermediate)

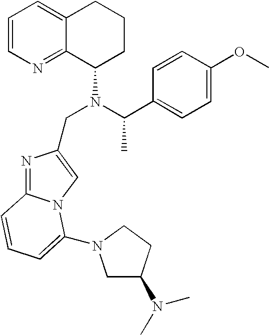

A solution of (8S)—N-[(5-fluoroimidazo[1,2-a]pyridin-2-yl)methyl]-N-{(1S)-1-[4-(methyloxy)phenyl]ethyl}-5,6,7,8-tetrahydro-8-quinolinamine (5.64 g, 13.1 mmol) in acetonitrile (65 mL) was treated with (3R)-(+)-3-dimethylaminopyrrolidine (7.48 g, 65.5 mmol) and heated at 70° C. for 15 hours. The reaction mixture was concentrated, diluted with dichloromethane, washed with saturated aqueous sodium carbonate, separated, and extracted with additional dichloromethane. The organic layers were combined, dried over magnesium sulfate, concentrated, and purified by flash chromatography (0-10% ammonium hydroxide in acetonitrile) to yield (8S)—N-({5-[(3R)-3-(dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine (5.94 g, 86% yield) as a pink solid. ¹H NMR (400 MHz, CDCl₃) δ 8.47 (d, J=4.2 Hz, 1H), 7.91 (s, 1H), 7.55 (d, J=8.5 Hz, 2H), 7.20 (d, J=7.4 Hz, 1H), 7.08-7.00 (m, 2H), 6.95 (dd, J=7.6, 4.7 Hz, 1H), 6.82-6.80 (m, 2H), 6.05 (d, J=6.7 Hz, 1H), 4.04 (m, 1H), 4.87 (m, 1H), 3.86 (m, 2H), 3.75 (s, 3H), 3.58-3.51 (m, 2H), 3.45 (m, 1H), 3.34 (m, 1H), 2.91 (m, 1H), 2.63 (m, 1H), 2.52 (m, 1H), 2.34 (s, 6H), 2.26 (m, 1H), 2.06-1.98 (m, 2H), 1.84-1.77 (m, 2H), 1.48 (m, 1H), 1.29 (d, J=7.1 Hz, 3H); MS m/z 525 (M+1).

### Example 36

### [5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol (Intermediate)

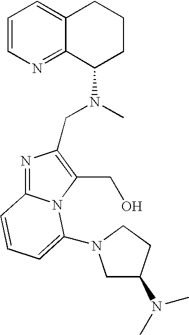

[5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-methyl-5,6,7,8-tetrahydro-8-quinolinamine (prepared from (8S)—N-({5-[(3R)-3-(Dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine by removal of methyloxyphenylethyl group and reductive amination in a similar fashion as described herein) via hydroxymethylation in a similar manner as shown herein to give a white solid (71% yield). ¹H NMR (400 MHz, CDCl₃) δ 8.40 (d, J=4.2 Hz, 1H), 7.32 (d, J=7.6 Hz, 1H), 7.27 (d, J=8.9 Hz, 1H), 7.06-6.99 (m, 2H), 6.42 (d, J=7.0 Hz, 1H), 5.16 (m, 2H), 4.07-3.95 (m, 3H), 3.69 (br, 1H), 3.43 (br, 2H), 3.17 (br, 1H), 3.04-2.97 (m, 2H), 2.77 (m, 1H), 2.65 (m, 1H), 2.28 (s, 6H), 2.24-2.20 (m, 2H), 2.15 (s, 2H), 2.02-1.89 (m, 3H), 1.67 (m, 1H); MS m/z 435 (M+1).

### Example 37

### 5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carbaldehyde (Intermediate)

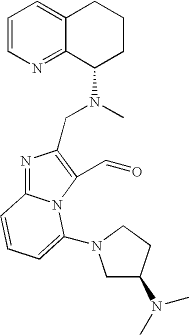

5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carbaldehyde was prepared from [5-[(3R)-3-(dimethylamino)-1-pyrrolidinyl]-2-({methyl[(8S)-5,6,7,8-tetrahydro-8-quinolinyl]amino}methyl)imidazo[1,2-a]pyridin-3-yl]methanol and IBX polystyrene in a similar manner as described herein to give a red solid (56% yield). ¹H NMR (400 MHz, CDCl₃) δ 10.45 (s, 1H), 8.47 (d, J=4.1 Hz, 1H), 7.37-7.34 (m, 3H), 7.04 (dd, J=7.5, 4.7 Hz, 1H), 6.59 (m, 1H), 4.26 (s, 2H), 4.19 (m, 1H), 3.48-3.37 (m, 2H), 3.04-2.81 (m, 3H), 2.69 (m, 1H), 2.51 (s, 3H), 2.28 (s, 6H), 2.22-2.16 (m, 2H), 2.11-2.04 (m, 2H), 1.92 (m, 1H), 1.76-1.66 (m, 2H); MS m/z 433 (M+1).

### Example 38

### 5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carbonitrile

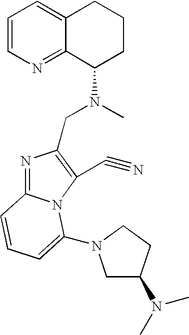

5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carbonitrile was prepared from 5-[(3R)-3-(dimethylamino)-1-pyrrolidinyl]-2-({methyl[(8S)-5,6,7,8-tetrahydro-8-quinolinyl]amino}methyl)imidazo[1,2-a]pyridine-3-carbaldehyde and hydroxylamine hydrochloride in a similar manner as described herein to give a yellow oil (77% yield). ¹H NMR (400 MHz, CDCl₃) δ 8.48 (d, J=4.5 Hz, 1H), 7.39 (d, J=8.8 Hz, 1H), 7.36-7.29 (m, 2H), 7.04 (dd, J=7.7, 4.7 Hz, 1H), 6.56 (d, J=7.1 Hz, 1H), 4.06 (m, 1H), 4.03 (s, 2H), 3.60 (m, 1H), 3.43 (m, 1H), 3.24-3.10 (m, 2H), 2.96 (m, 1H), 2.85 (m, 1H), 2.68 (m, 1H), 2.46 (s, 3H), 2.31-2.26 (m, 7H), 2.15-1.95 (m, 4H), 1.71 (m, 1H); MS m/z 430 (M+1).

### Example 39

### 5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carboxamide

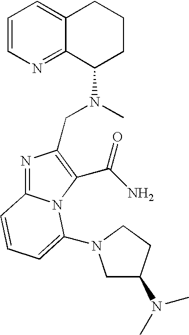

5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carboxamide was prepared from 5-[(3R)-3-(dimethylamino)-1-pyrrolidinyl]-2-({methyl[(8S)-5,6,7,8-tetrahydro-8-quinolinyl]amino}methyl)imidazo[1,2-a]pyridine-3-carbonitrile and sulfuric acid in a similar manner as described herein to give an orange solid (52% yield). ¹H NMR (400 MHz, CDCl₃) δ 10.46 (s, 1H), 8.35 (m, 1H), 7.31 (d, J=7.6 Hz, 1H), 7.24-7.20 (m, 1H), 7.13-7.11 (m, 1H), 7.00 (dd, J=7.9, 4.6 Hz, 1H), 6.25 (d, J=7.4 Hz, 1H), 5.63 (s, 1H), 4.12 (d, J=12.7 Hz, 1H), 4.04 (d, J=13.0 Hz, 1H), 4.00 (m, 1H), 3.63-3.43 (m, 2H), 3.15 (m, 1H), 3.02-2.92 (m, 2H), 2.80-2.63 (m, 2H), 2.29 (s, 3H), 2.27 (s, 3H), 2.23-2.19 (m, 2H), 2.06 (s, 3H), 2.02-1.94 (m, 2H), 1.83 (m, 1H), 1.68 (m, 1H); MS m/z 448 (M+1). 3H)

### Example 40

### [5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridin-3-yl]methanol (Intermediate)

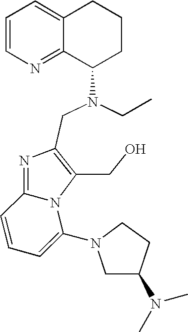

[5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-ethyl-5,6,7,8-tetrahydro-8-quinolinamine via hydroxymethylation in a similar manner as shown herein to give a yellow oil (28% yield). ¹H NMR (400 MHz, CDCl₃) δ 8.34 (d, J=4.4 Hz, 1H), 7.29-7.26 (m, 2H), 7.04 (m, 1H), 6.97 (dd, J=7.6, 4.7 Hz, 1H), 6.42 (d, J=7.2 Hz, 1H), 5.29-5.25 (m, 1H), 5.18-5.15 (m, 1H), 4.18 (d, J=13.2 Hz, 1H), 3.89-3.85 (m, 2H), 3.26 (br, 1H), 3.06 (m, 1H), 2.79-2.71 (m, 2H), 2.62 (m, 1H), 2.47 (m, 1H), 2.40-2.32 (m, 2H), 2.29 (s, 6H), 2.24-2.19 (m, 2H), 1.99-1.92 (m, 3H), 1.79 (m, 1H), 1.59 (m, 1H), 1.13 (t, J=7.0 Hz, 3H); MS m/z 449 (M+1).

### Example 41

### 5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carbaldehyde (Intermediate)

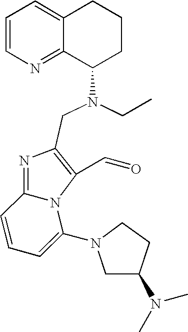

5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carbaldehyde was prepared from [5-[(3R)-3-(dimethylamino)-1-pyrrolidinyl]-2-({ethyl[(8S)-5,6,7,8-tetrahydro-8-quinolinyl]amino}methyl)imidazo[1,2-a]pyridin-3-yl]methanol and IBX polystyrene in a similar manner as described herein to give a red solid (41% yield). ¹H NMR (400 MHz, CDCl₃) δ 10.49 (s, 1H), 8.44 (d, J=4.0 Hz, 1H), 7.38-7.31 (m, 3H), 7.00 (dd, J=7.5, 4.8 Hz, 1H), 6.56 (d, J=7.4 Hz, 1H), 4.34-4.21 (m, 3H), 3.44-3.36 (m, 2H), 3.12-2.79 (m, 6H), 2.28 (s, 6H), 2.22-2.15 (m, 2H), 2.04-1.87 (m, 3H), 1.72-1.63 (m, 2H), 1.00 (t, J=7.6 Hz, 3H); MS m/z 447 (M+1).

### Example 42

### 5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carbonitrile

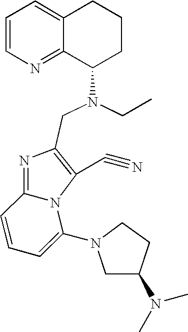

5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carbonitrile was prepared from 5-[(3R)-3-(dimethylamino)-1-pyrrolidinyl]-2-({ethyl[(8S)-5,6,7,8-tetrahydro-8-quinolinyl]amino}methyl)imidazo[1,2-a]pyridine-3-carbaldehyde and hydroxylamine hydrochloride in a similar manner as described herein to give a yellow oil (68% yield). ¹H NMR (400 MHz, CDCl₃) δ 8.43 (d, J=4.5 Hz, 1H), 7.37 (d, J=8.9 Hz, 1H), 7.31-7.27 (m, 2H), 6.99 (dd, J=7.7, 4.7 Hz, 1H), 6.54 (d, J=7.2 Hz, 1H), 4.19 (m, 1H), 4.13 (d, J=14.5 Hz, 1H), 3.95 (d, J=14.5 Hz, 1H), 3.57 (m, 1H), 3.41 (m, 1H), 3.22-3.11 (m, 2H), 2.98-2.92 (m, 2H), 2.84-2.75 (m, 2H), 2.65 (m, 1H), 2.31 (m, 1H), 2.28 (s, 6H), 2.14 (m, 1H), 2.04-1.91 (m, 3H), 1.66 (m, 1H), 1.08 (m, 3H); MS m/z 444 (M+1).

### Example 43

### 5-[(3R)-3-(Dimethylamino)-1-pyrrolidinyl]-2-({ethyl[(8S)-5,6,7,8-tetrahydro-8-quinolinyl]amino}methyl)imidazo[1,2-d]pyridine-3-carboxamide

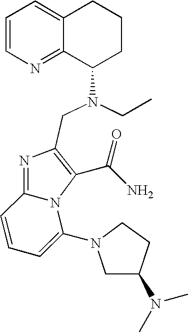

5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carboxamide was prepared from 5-[(3R)-3-(dimethylamino)-1-pyrrolidinyl]-2-({ethyl[(8S)-5,6,7,8-tetrahydro-8-quinolinyl]amino}methyl)imidazo[1,2-a]pyridine-3-carbonitrile and sulfuric acid in a similar manner as described herein to give an pale orange solid (37% yield). ¹H NMR (400 MHz, CDCl₃) δ 10.58 (s, 1H), 8.33 (m, 1H), 7.30-7.21 (m, 2H), 7.14 (m, 1H), 6.98 (m, 1H), 6.26 (m, 1H), 5.62 (s, 1H), 4.24 (m, 1H), 4.07-3.99 (m, 2H), 3.68 (m, 1H), 3.47 (m, 1H), 3.27 (m, 1H), 3.13 (m, 1H), 2.97 (m, 1H), 2.82-2.72 (m, 2H), 2.64 (m, 1H), 2.57-2.50 (m, 2H), 2.30-2.24 (m, 7H), 1.99-1.83 (m, 3H), 1.64 (m, 1H), 0.91-0.86 (m, 3H); MS m/z 462 (M+1).

### Example 44

### [5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol (Intermediate)

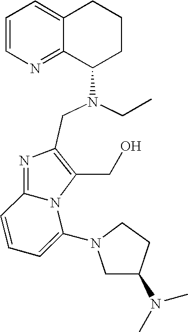

[5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-propyl-5,6,7,8-tetrahydro-8-quinolinamine via hydroxymethylation in a similar manner as shown herein to give a yellow oil (34% yield). ¹H NMR (400 MHz, CDCl₃) δ 8.32 (d, J=4.3 Hz, 1H), 7.28-7.26 (m, 2H), 7.04 (m, 1H), 6.96 (dd, J=7.7, 4.9 Hz, 1H), 6.43 (d, J=7.0 Hz, 1H), 5.27-5.24 (m, 1H), 5.16-5.13 (m, 1H), 4.19 (d, J=13.5 Hz, 1H), 3.90-3.82 (m, 2H), 3.24 (br, 1H), 3.06 (m, 1H), 2.79-2.70 (m, 2H), 2.60 (m, 1H), 2.43-2.32 (m, 2H), 2.28 (s, 6H), 2.22-2.15 (m, 3H), 1.98-1.91 (m, 3H), 1.80 (m, 1H), 1.63-1.54 (m, 3H), 0.77 (t, J=7.3 Hz, 3H); MS m/z 463 (M+1).

### Example 45

### 5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carbaldehyde (Intermediate)

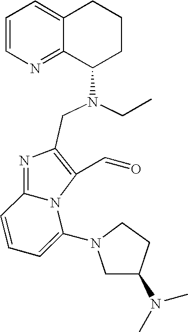

5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carbaldehyde was prepared [5-[(3R)-3-(dimethylamino)-1-pyrrolidinyl]-2-({propyl[(8S)-5,6,7,8-tetrahydro-8-quinolinyl]amino}methyl)imidazo[1,2-a]pyridin-3-yl]methanol and IBX polystyrene in a similar manner as described herein to give a red solid (42% yield). ¹H NMR (400 MHz, CDCl₃) δ 10.51 (s, 1H), 8.44 (d, J=4.1 Hz, 1H), 7.39-7.30 (m, 3H), 7.00 (dd, J=7.5, 4.6 Hz, 1H), 6.55 (d, J=7.4 Hz, 1H), 4.39-4.20 (m, 3H), 3.47-3.37 (m, 4H), 3.01-2.91 (m, 2H), 2.86-2.77 (m, 2H), 2.70-2.62 (m, 2H), 2.28 (s, 6H), 2.23-2.13 (m, 2H), 1.99-1.87 (m, 2H), 1.71-1.64 (m, 2H), 1.47 (m, 1H), 0.78 (m, 3H); MS m/z 461 (M+1).

### Example 46

### 5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carbonitrile

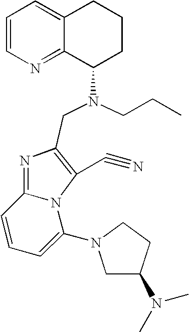

5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carbonitrile was prepared from 5-[(3R)-3-(dimethylamino)-1-pyrrolidinyl]-2-({propyl[(8S)-5,6,7,8-tetrahydro-8-quinolinyl]amino}methyl)imidazo[1,2-a]pyridine-3-carbaldehyde and hydroxylamine hydrochloride in a similar manner as described herein to give a yellow oil (67% yield). ¹H NMR (400 MHz, CDCl₃) δ 8.43 (d, J=4.5 Hz, 1H), 7.39 (d, J=8.5 Hz, 1H), 7.33-7.29 (m, 2H), 7.00 (dd, J=7.8, 4.6 Hz, 1H), 6.55 (d, J=7.3 Hz, 1H), 4.24 (d, J=14.8 Hz, 1H), 4.18 (m, 1H), 4.00 (d, J=15.4 Hz, 1H), 3.59 (m, 1H), 3.44 (m, 1H), 3.24-3.13 (m, 2H), 2.98 (m, 1H), 2.88-2.77 (m, 2H), 2.68-2.57 (m, 2H), 2.33 (m, 1H), 2.30 (s, 6H), 2.17 (m, 1H), 2.05-1.94 (m, 3H), 1.68 (m, 1H), 1.54-1.44 (m, 2H), 0.79 (t, J=7.3 Hz, 3H); MS m/z 458 (M+1).

### Example 47

### 5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carboxamide

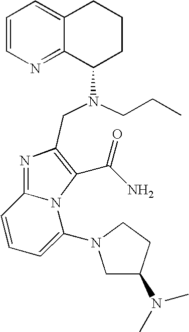

5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carboxamide was prepared from 5-[(3R)-3-(dimethylamino)-1-pyrrolidinyl]-2-({propyl[(8S)-5,6,7,8-tetrahydro-8-quinolinyl]amino}methyl)imidazo[1,2-a]pyridine-3-carbonitrile and sulfuric acid in a similar manner as described herein to give an dark orange solid (39% yield). ¹H NMR (400 MHz, CDCl₃) δ10.49 (s, 1H), 8.33 (d, J=4.5 Hz, 1H), 7.29 (d, J=7.6 Hz, 1H), 7.26-7.21 (m, 1H), 7.13 (d, J=8.7 Hz, 1H), 6.99 (dd, J=7.6, 4.6 Hz, 1H), 6.26 (d, J=7.6 Hz, 1H), 5.61 (s, 1H), 4.21 (d, J=13.2 Hz, 1H), 4.06 (d, J=13.0 Hz, 1H), 4.02 (m, 1H), 3.66 (m, 1H), 3.47 (m, 1H), 3.26 (m, 1H), 2.96 (m, 1H), 2.82-2.73 (m, 2H), 2.64 (m, 1H), 2.48-2.37 (m, 2H), 2.28 (s, 6H), 2.24-2.18 (m, 2H), 1.99-1.84 (m, 3H), 1.64 (m, 1H), 1.32-1.24 (m, 2H), 0.65 (t, J=7.3 Hz, 3H); MS m/z 476 (M+1).

### Example 48

### [5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol (Intermediate)

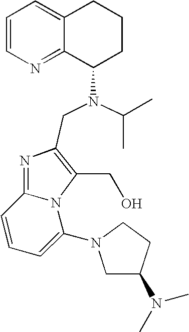

[5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-(1-methylethyl)-5,6,7,8-tetrahydro-8-quinolinamine via hydroxymethylation in a similar manner as shown herein to give a yellow oil (32% yield). ¹H NMR (400 MHz, CDCl₃) δ 8.31 (m, 1H), 7.29-7.27 (m, 2H), 7.05 (m, 1H), 6.97 (m, 1H), 6.44 (m, 1H), 5.31-5.28 (m, 1H), 5.15-5.12 (m, 1H), 4.18 (m, 1H), 4.00 (m, 1H), 3.85 (m, 1H), 3.21 (br, 1H), 3.10 (m, 1H), 2.83-2.75 (m, 3H), 2.62 (m, 1H), 2.46 (m, 1H), 2.30 (s, 6H), 2.24-2.20 (m, 2H), 2.01-1.93 (m, 3H), 1.83 (m, 1H), 1.58 (m, 1H), 1.20 (m, 6H); MS m/z 463 (M+1).

### Example 49

### 5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbaldehyde (Intermediate)

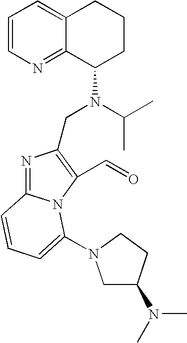

5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbaldehyde was prepared [5-[(3R)-3-(dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol and IBX polystyrene in a similar manner as described herein to give a red solid (25% yield). ¹H NMR (400 MHz, CDCl₃) δ 10.53 (s, 1H), 8.45 (br, 1H), 7.39-7.34 (m, 3H), 6.53 (m, 1H), 6.97 (m, 1H), 4.36-4.30 (m, 3H), 3.44-3.29 (m, 4H), 3.12-2.93 (m, 2H), 2.80 (m, 1H), 2.64 (m, 1H), 2.29 (s, 6H), 2.21-1.89 (m, 4H), 1.74-1.64 (m, 2H), 1.20-1.15 (m, 6H); MS m/z 461 (M+1).

### Example 50

### 5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbonitrile

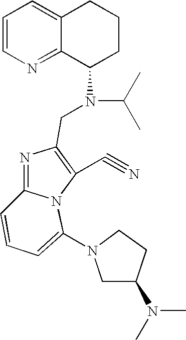

5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbonitrile was prepared from 5-[(3R)-3-(dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbaldehyde and hydroxylamine hydrochloride in a similar manner as described herein to give a yellow oil (58% yield). ¹H NMR (400 MHz, CDCl₃) δ 8.40 (d, J=4.4 Hz, 1H), 7.34 (d, J=8.5 Hz, 1H), 7.30-7.27 (m, 1H), 7.23-7.21 (m, 1H), 6.93 (dd, J=7.6, 4.6 Hz, 1H), 6.52 (d, J=7.1 Hz, 1H), 4.22 (m, 1H), 4.13 (d, J=15.6 Hz, 1H), 3.91 (d, J=15.9 Hz, 1H), 3.57 (m, 1H), 3.42 (m, 1H), 3.31 (m, 1H), 3.21-3.13 (m, 2H), 2.96 (m, 1H), 2.79 (m, 1H), 2.62 (m, 1H), 2.31 (s, 6H), 2.26 (m, 1H), 2.11-2.02 (m, 4H), 1.68 (m, 1H), 1.18-1.15 (m, 6H); MS m/z 458 (M+1).

### Example 51

### 5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carboxamide

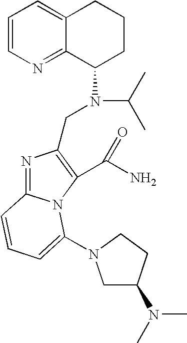

5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carboxamide was prepared from 5-[(3R)-3-(dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbonitrile and sulfuric acid in a similar manner as described herein to give an orange solid (48% yield). ¹H NMR (400 MHz, CDCl₃) δ 10.53 (s, 1H), 8.29 (d, J=4.5 Hz, 1H), 7.26-7.19 (m, 2H), 7.14 (d, J=8.7 Hz, 1H), 6.94 (dd, J=7.6, 4.5 Hz, 1H), 6.25 (d, J=7.2 Hz, 1H), 5.64 (s, 1H), 4.31 (d, J=13.3 Hz, 1H), 4.06 (d, J=13.1 Hz, 1H), 3.97 (m, 1H), 3.65 (m, 1H), 3.40 (m, 1H), 3.22 (m, 1H), 2.98-2.86 (m, 2H), 2.79-2.59 (m, 3H), 2.32 (m, 1H), 2.26 (s, 6H), 2.20 (m, 1H), 2.03-1.94 (m, 2H), 1.84 (m, 1H), 1.61 (m, 1H), 1.20 (d, J=6.6 Hz, 3H), 0.97 (d, J=6.6 Hz, 3H); MS m/z 476 (M+1).

### Example 52

### [5-(4-Methyl-1-piperazinyl)-2-({(phenylmethyl)[(8S)-5,6,7,8-tetrahydro-8-quinolinyl]amino}methyl)imidazo[1,2-a]pyridin-3-yl]methanol (Intermediate)

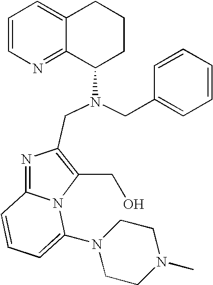

[5-(4-Methyl-1-piperazinyl)-2-({(phenylmethyl)[(8S)-5,6,7,8-tetrahydro-8-quinolinyl]amino}methyl)imidazo[1,2-a]pyridin-3-yl]methanol was prepared from (8S)—N-{[5-(4-methyl-1-piperazinyl)imidazo[1,2-a]pyridin-2-yl]methyl}-N-(phenylmethyl)-5,6,7,8-tetrahydro-8-quinolinamine via hydroxymethylation in a similar manner as shown herein to give an off-white solid (42% yield). ¹H NMR (400 MHz, CDCl₃) δ 8.51 (d, J=4.5 Hz, 1H), 7.53 (d, J=7.3 Hz, 2H), 7.37-7.29 (m, 3H), 7.27-7.23 (m, 1H), 7.07-7.01 (m, 3H), 6.41 (d, J=7.2 Hz, 1H), 5.33 (d, J=12.2 Hz, 1H), 4.75 (d, J=13.2 Hz, 1H), 4.02 (d, J=13.4 Hz, 1H), 3.90 (m, 1H), 3.82 (d, J=13.2 Hz, 1H), 3.74 (m, 1H), 3.56 (d, J=13.0 Hz, 1H), 3.34 (d, J=12.4 Hz, 1H), 3.03 (m, 1H), 2.95-2.90 (m, 2H), 2.87-2.75 (m, 3H), 2.66-2.57 (m, 2H), 2.48-2.44 (m, 2H), 2.41 (s, 3H), 2.02-1.94 (m, 2H), 1.61 (m, 1H); MS m/z 497 (M+1).

### Example 53

### 5-[(3R)-3-(Dimethylamino)-1-pyrrolidinyl]-2-({(phenylmethyl)[(8S)-5,6,7,8-tetrahydro-8-quinolinyl]amino}methyl)imidazo[1,2-a]pyridine-3-carbonitrile

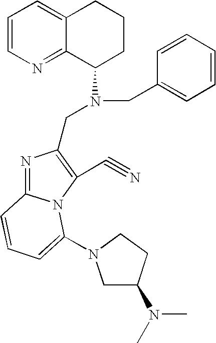

5-[(3R)-3-(Dimethylamino)-1-pyrrolidinyl]-2-({(phenylmethyl)[(8S)-5,6,7,8-tetrahydro-8-quinolinyl]amino}methyl)imidazo[1,2-a]pyridine-3-carbaldehyde was prepared [5-(4-methyl-1-piperazinyl)-2-({(phenylmethyl)[(8S)-5,6,7,8-tetrahydro-8-quinolinyl]amino}methyl)imidazo[1,2-a]pyridin-3-yl]methanol and IBX polystyrene in a similar manner as described herein to give a tan solid (30% yield). 5-[(3R)-3-(Dimethylamino)-1-pyrrolidinyl]-2-({(phenylmethyl)[(8S)-5,6,7,8-tetrahydro-8-quinolinyl]amino}methyl)imidazo[1,2-a]pyridine-3-carbonitrile was prepared from 5-[(3R)-3-(dimethylamino)-1-pyrrolidinyl]-2-({(phenylmethyl)[(8S)-5,6,7,8-tetrahydro-8-quinolinyl]amino}methyl)imidazo[1,2-a]pyridine-3-carbaldehyde and hydroxylamine hydrochloride in a similar manner as described herein to give a brown solid (44% yield). ¹H NMR (400 MHz, CDCl₃) δ 8.50 (m, 1H), 7.44-7.42 (m, 2H), 7.38-7.29 (m, 3H), 7.16-7.12 (m, 2H), 7.06-7.01 (m, 2H), 6.54 (d, J=7.4 Hz, 1H), 4.22-4.07 (m, 3H), 3.85 (d, J=14.4 Hz, 2H), 3.54 (m, 1H), 3.38 (m, 1H), 3.22-3.13 (m, 2H), 2.97 (m, 1H), 2.79 (m, 1H), 2.69-2.62 (m, 2H), 2.35 (m, 1H), 2.33 (s, 6H), 2.11-1.97 (m, 3H), 1.66 (m, 1H); MS m/z 506 (M+1).

### Example 54

### (8S)—N-({5-[4-(dimethylamino)-1-piperidinyl]imidazo[1,2-a]pyridin-2-yl}methyl)-N-{(1S)-1-[4-(methyloxy)phenyl]ethyl}-5,6,7,8-tetrahydro-8-quinolinamine (Intermediate)

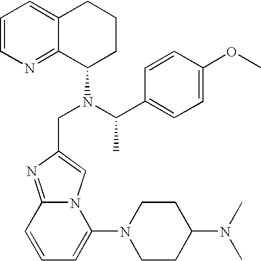

(8S)—N-({5-[4-(dimethylamino)-1-piperidinyl]imidazo[1,2-a]pyridin-2-yl}methyl)-N-{(1S)-1-[4-(methyloxy)phenyl]ethyl}-5,6,7,8-tetrahydro-8-quinolinamine was prepared from (8S)—N-[(5-fluoroimidazo[1,2-a]pyridin-2-yl)methyl]-N-{(1S)-1-[4-(methyloxy)phenyl]ethyl}-5,6,7,8-tetrahydro-8-quinolinamine and N,N-dimethyl-4-piperidinamine in a similar fashion as described herein to yield a pink solid (74% yield). ¹H NMR (400 MHz, CDCl₃) δ 8.47 (d, 1H), 7.72 (s, 1H), 7.58 (d, 2H), 7.19 (m, 2H), 7.05 (t, 1H), 6.95 (dd, 1H), 6.83 (m, 2H), 6.16 (d, 1H), 4.87 (q, 1H), 4.04 (m, 1H), 3.96 (m, 1H), 3.76 (s, 3H), 3.62-3.47 (m, 2H), 2.81-2.49 (m, 5H), 2.41 (s, 6H), 2.35 (m, 1H), 2.07 (m, 3H), 1.92-1.75 (m, 4H), 1.51 (m, 1H), 1.31 (d, 3H); MS m/z 539 (M+1).

### Example 55

### (8S)—N-({5-[4-(dimethylamino)-1-piperidinyl]imidazo[1,2-a]pyridin-2-yl}methyl)-5,6,7,8-tetrahydro-8-quinolinamine (Intermediate)

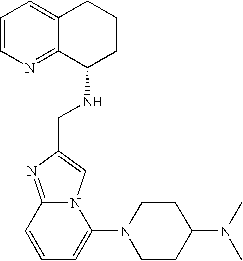

(8S)—N-({5-[4-(dimethylamino)-1-piperidinyl]imidazo[1,2-a]pyridin-2-yl}methyl)-5,6,7,8-tetrahydro-8-quinolinamine was prepared from (8S)—N-({5-[4-(dimethylamino)-1-piperidinyl]imidazo[1,2-a]pyridin-2-yl}methyl)-N-{(1S)-1-[4-(methyloxy)phenyl]ethyl}-5,6,7,8-tetrahydro-8-quinolinamine and trifluoroacetic acid in a similar fashion as described herein to obtain a yellow foam (87% yield). ¹H NMR (400 MHz, CDCl₃) 8.41 (d, 1H), 7.57 (s, 1H), 7.37 (d, 1H), 7.29 (d, 1H), 7.12 (m, 1H), 7.05 (dd, 1H), 6.22 (d, 1H), 4.16 (q, 2H), 3.96 (dd, 1H), 3.52 (m, 2H), 2.89-2.65 (m, 4H), 2.35 (s, 6H), 2.26-2.18 (m, 2H), 2.08-1.85 (m, 5H), 1.82-1.69 (m, 3H); MS m/z 405 (M+1).

### Example 56

### (8S)—N-({5-[4-(dimethylamino)-1-piperidinyl]imidazo[1,2-a]pyridin-2-yl}methyl)-N-methyl-5,6,7,8-tetrahydro-8-quinolinamine (Intermediate)

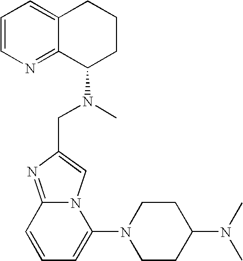

(8S)—N-({5-[4-(dimethylamino)-1-piperidinyl]imidazo[1,2-a]pyridin-2-yl}methyl)-N-methyl-5,6,7,8-tetrahydro-8-quinolinamine was prepared from (8S)—N-({5-[4-(dimethylamino)-1-piperidinyl]imidazo[1,2-a]pyridin-2-yl}methyl)-5,6,7,8-tetrahydro-8-quinolinamine and formaldehyde under reductive amination conditions as described herein to obtain a yellow oil (61% yield). ¹H-NMR (CDCl₃): δ 8.50 (d, 1H), 7.64 (s, 1H), 7.32 (d, 1H), 7.25 (m, 1H), 7.05 (m, 2H), 6.19 (dd, 1H), 4.10 (m, 1H), 3.93 (s, 2H), 3.50 (m, 2H), 2.86-2.75 (m, 1H), 2.72-2.60 (m, 3H), 2.37 (s, 3H), 2.34 (s, 6H), 2.17-1.60 (m, 9H); MS m/z 419 (M+1).

### Example 57

### [5-[4-(dimethylamino)-1-piperidinyl]-2-({methyl[(8S)-5,6,7,8-tetrahydro-8-quinolinyl]amino}methyl)imidazo[1,2-a]pyridin-3-yl]methanol (Intermediate)

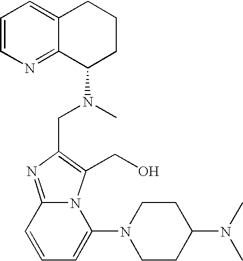

[5-[4-(dimethylamino)-1-piperidinyl]-2-({methyl[(8S)-5,6,7,8-tetrahydro-8-quinolinyl]amino}methyl)imidazo[1,2-a]pyridin-3-yl]methanol was prepared from (8S)—N-({5-[4-(dimethylamino)-1-piperidinyl]imidazo[1,2-a]pyridin-2-yl}methyl)-N-methyl-5,6,7,8-tetrahydro-8-quinolinamine via hydroxymethylation in a similar manner as described herein to give a white solid (56% yield). ¹H-NMR (CDCl₃): δ 8.45 (d, 1H), 7.33 (m, 2H), 7.05 (m, 2H), 6.37 (d, 1H), 5.27 (m, 2H), 4.03 (m, 3H), 3.65 (m, 1H), 3.53 (m, 1H), 2.80 (m, 1H), 2.72-2.61 (m, 3H), 2.43-2.35 (m, 7H), 2.23 (m, 1H), 2.17 (s, 3H), 2.07-1.81 (m, 7H), 1.71 (m, 1H); MS m/z 449 (M+1).

### Example 58

### 5-[4-(dimethylamino)-1-piperidinyl]-2-({methyl[(8S)-5,6,7,8-tetrahydro-8-quinolinyl]amino}methyl)imidazo[1,2-a]pyridine-3-carbonitrile

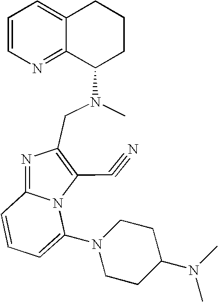

5-[4-(dimethylamino)-1-piperidinyl]-2-({methyl[(8S)-5,6,7,8-tetrahydro-8-quinolinyl]amino}methyl)imidazo[1,2-a]pyridine-3-carbonitrile was prepared from an IBX polystyrene resin oxidation of [5-[4-(dimethylamino)-1-piperidinyl]-2-({methyl[(8S)-5,6,7,8-tetrahydro-8-quinolinyl]amino}methyl)imidazo[1,2-a]pyridin-3-yl]methanol as described herein, then carried forward crude to reaction with hydroxylamine hydrochloride in a similar manner as described herein to obtain a tan solid (76%). ¹H-NMR (CDCl₃): δ 8.47 (d, 1H), 7.43 (m, 1H), 7.33 (m, 2H), 7.04 (dd, 1H), 6.51 (m, 1H), 4.06 (m, 3H), 3.46 (m, 2H), 2.85 (m, 1H), 2.71 (m, 3H), 2.44 (m, 3H), 2.35 (s, 6H), 2.21-2.00 (m, 6H), 1.84 (m, 2H), 1.72 (m, 1H); MS m/z 444 (M+1).

### Example 59

### 5-[4-(dimethylamino)-1-piperidinyl]-2-({methyl)[(8S)-5,6,7,8-tetrahydro-8-quinolinyl]amino}methyl)imidazo[1,2-a]pyridine-3-carboxamide

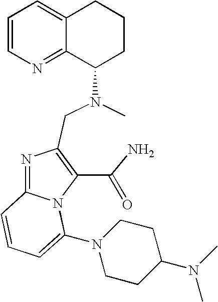

5-[4-(dimethylamino)-1-piperidinyl]-2-({methyl[(8S)-5,6,7,8-tetrahydro-8-quinolinyl]amino}methyl)imidazo[1,2-a]pyridine-3-carboxamide was prepared from 5-[4-(dimethylamino)-1-piperidinyl]-2-({methyl[(8S)-5,6,7,8-tetrahydro-8-quinolinyl]amino}methyl)imidazo[1,2-a]pyridine-3-carbonitrile and sulfuric acid as described herein to give a tan solid (46% yield). ¹H-NMR (CDCl₃): δ 10.18 (s, 1H), 8.36 (d, 1H), 7.31 (m, 1H), 7.21 (m, 2H), 7.00 (dd, 1H), 6.32 (m, 1H), 5.74 (s, 1H), 4.11 (m, 1H), 3.95 (m, 2H), 3.54 (m, 2H), 2.77 (m, 1H), 2.70-2.41 (m, 4H), 2.35 (s, 6H), 2.10 (m, 4H), 2.05-1.80 (m, 6H), 1.69 (m, 1H); MS m/z 462 (M+1).

### Example 60

### (8S)—N-({5-[4-(dimethylamino)-1-piperidinyl]imidazo[1,2-a]pyridin-2-yl}methyl)-N-ethyl-5,6,7,8-tetrahydro-8-quinolinamine (Intermediate)

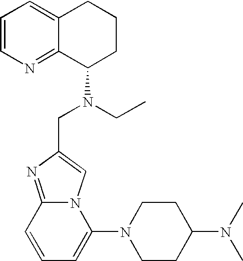

(8S)—N-({5-[4-(dimethylamino)-1-piperidinyl]imidazo[1,2-a]pyridin-2-yl}methyl)-N-ethyl-5,6,7,8-tetrahydro-8-quinolinamine was prepared from (8S)—N-({5-[4-(dimethylamino)-1-piperidinyl]imidazo[1,2-a]pyridin-2-yl}methyl)-5,6,7,8-tetrahydro-8-quinolinamine and acetaldehyde under reductive amination conditions as described herein to obtain a yellow oil (13% yield). ¹H-NMR (CDCl₃): δ 8.48 (d, 1H), 7.61 (s, 1H), 7.30 (d, 1H), 7.24 (m, 1H), 7.06 (m, 2H), 6.19 (m, 1H), 4.19 (m, 1H), 3.98 (m, 1H), 3.86 (m, 1H), 3.51 (m, 2H), 2.95-2.60 (m, 4H), 2.41-2.33 (m, 8H), 2.18-1.58 (m, 9H), 1.07 (t, 3H); MS m/z 433 (M+1).

### Example 61

### [5-[4-(dimethylamino)-1-piperidinyl]-2-({ethyl[(8S)-5,6,7,8-tetrahydro-8-quinolinyl]amino}methyl)imidazo[1,2-a]pyridin-3-yl]methanol (Intermediate)

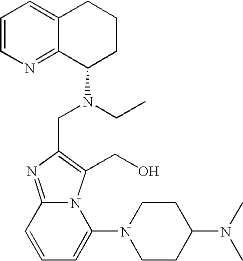

[5-[4-(dimethylamino)-1-piperidinyl]-2-({ethyl[(8S)-5,6,7,8-tetrahydro-8-quinolinyl]amino}methyl)imidazo[1,2-a]pyridin-3-yl]methanol was prepared from (8S)—N-({5-[4-(dimethylamino)-1-piperidinyl]imidazo[1,2-a]pyridin-2-yl}methyl)-N-ethyl-5,6,7,8-tetrahydro-8-quinolinamine via hydroxymethylation in a similar manner as described herein to give a white solid (61% yield). ¹H-NMR (CDCl₃): δ 8.39 (d, 1H), 7.30 (m, 2H), 7.02 (m, 2H), 6.37 (d, 1H), 5.45 (m, 1H), 5.20 (m, 1H), 4.19 (m, 1H), 3.89 (m, 3H), 3.37 (m, 1H), 2.82-2.29 (m, 14H), 1.98 (m, 4H), 1.84 (m, 3H), 1.63 (m, 1H), 1.13 (t, 3H); MS m/z 463 (M+1).

### Example 62

### 5-[4-(dimethylamino)-1-piperidinyl]-2-({ethyl[(8S)-5,6,7,8-tetrahydro-8-quinolinyl]amino}methyl)imidazo[1,2-a]pyridine-3-carbonitrile

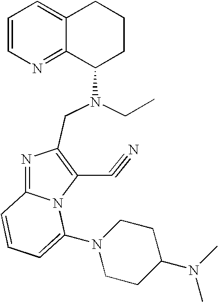

5-[4-(dimethylamino)-1-piperidinyl]-2-({ethyl[(8S)-5,6,7,8-tetrahydro-8-quinolinyl]amino}methyl)imidazo[1,2-a]pyridine-3-carbonitrile was prepared from [5-[4-(dimethylamino)-1-piperidinyl]-2-({ethyl[(8S)-5,6,7,8-tetrahydro-8-quinolinyl]amino}methyl)imidazo[1,2-a]pyridin-3-yl]methanol from an IBX polystyrene resin oxidation as described herein, then carried forward crude to reaction with hydroxylamine hydrochloride in a similar manner as described herein to obtain a tan solid (43%). ¹H-NMR (CDCl₃): δ 8.43 (d, 1H), 7.42 (m, 1H), 7.31 (m, 2H), 6.99 (dd, 1H), 6.50 (m, 1H), 4.17 (m, 2H), 3.92 (m, 1H), 3.45 (m, 2H), 2.91 (m, 1H), 2.83-2.56 (m, 6H), 2.41 (s, 6H), 2.26-2.12 (m, 3H), 2.06-1.83 (m, 4H), 1.67 (m, 1H), 1.08 (t, 3H); MS m/z 458 (M+1).

### Example 63

### 5-[4-(dimethylamino)-1-piperidinyl]-2-({ethyl[(8S)-5,6,7,8-tetrahydro-8-quinolinyl]amino}methyl)imidazo[1,2-a]pyridine-3-carboxamide

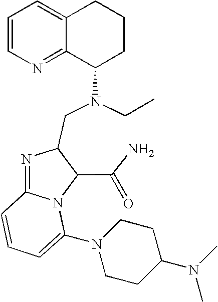

5-[4-(dimethylamino)-1-piperidinyl]-2-({ethyl[(8S)-5,6,7,8-tetrahydro-8-quinolinyl]amino}methyl)imidazo[1,2-a]pyridine-3-carboxamide was prepared from 5-[4-(dimethylamino)-1-piperidinyl]-2-({ethyl[(8S)-5,6,7,8-tetrahydro-8-quinolinyl]amino}methyl)imidazo[1,2-a]pyridine-3-carbonitrile and sulfuric acid as described herein to give a tan solid (32% yield). ¹H-NMR (CDCl₃): δ 10.42 (s, 1H), 8.31 (d, 1H), 7.22 (m, 3H), 6.96 (dd, 1H), 6.30 (m, 1H), 5.72 (s, 1H), 4.20 (m, 1H), 3.96 (m, 1H), 3.87 (t, 1H), 3.50 (m, 2H), 2.81-2.47 (m, 5H), 2.34 (s, 6H), 2.21 (m, 1H), 2.05-1.83 (m, 7H), 1.78 (m, 1H), 1.61 (m, 1H), 0.93 (t, 3H); MS m/z 476 (M+1).

### Example 64

### (8S)—N-({5-[4-(dimethylamino)-1-piperidinyl]imidazo[1,2-a]pyridin-2-yl}methyl)-N-propyl-5,6,7,8-tetrahydro-8-quinolinamine

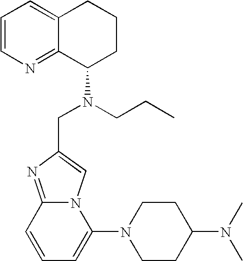

(8S)—N-({5-[4-(dimethylamino)-1-piperidinyl]imidazo[1,2-a]pyridin-2-yl}methyl)-N-propyl-5,6,7,8-tetrahydro-8-quinolinamine was prepared from (8S)—N-({5-[4-(dimethylamino)-1-piperidinyl]imidazo[1,2-a]pyridin-2-yl}methyl)-5,6,7,8-tetrahydro-8-quinolinamine and propionaldehyde under reductive amination conditions as described herein to obtain a yellow oil (59% yield). ¹H-NMR (CDCl₃): δ 8.47 (d, 1H), 7.63 (s, 1H), 7.29 (d, 1H), 7.23 (d, 1H), 7.07 (m, 1H), 6.99 (m, 1H), 6.17 (d, 1H), 4.17 (m, 1H), 4.03 (m, 1H), 3.87 (m, 1H), 3.52 (m, 2H), 2.83-2.59 (m, 4H), 2.37 (s, 6H), 2.12 (m, 2H), 2.04-1.73 (m, 9H), 1.48 (qt, 2H), 0.83 (t, 3H); MS m/z 447 (M+1).

### Example 65

### [5-[4-(dimethylamino)-1-piperidinyl]-2-({propyl[(8S)-5,6,7,8-tetrahydro-8-quinolinyl]amino}methyl)imidazo[1,2-a]pyridin-3-yl]methanol (Intermediate)

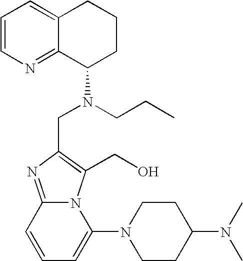

[5-[4-(dimethylamino)-1-piperidinyl]-2-({propyl[(8S)-5,6,7,8-tetrahydro-8-quinolinyl]amino}methyl)imidazo[1,2-a]pyridin-3-yl]methanol was prepared from (8S)—N-({5-[4-(dimethylamino)-1-piperidinyl]imidazo[1,2-a]pyridin-2-yl}methyl)-N-propyl-5,6,7,8-tetrahydro-8-quinolinamine via hydroxymethylation in a similar manner as described herein to give a white solid (52% yield). ¹H-NMR (CDCl₃): δ 8.38 (d, 1H), 7.30 (m, 2H), 7.06 (m, 1H), 6.99 (m, 1H), 6.37 (d, 1H), 5.45 (m, 1H), 5.18 (m, 1H), 4.21 (m, 1H), 3.88 (m, 3H), 3.35 (m, 1H), 2.82-2.53 (m, 4H), 2.48-2.20 (m, 10H), 2.03-1.92 (m, 4H), 1.84 (m, 3H), 1.59 (m, 3H), 0.79 (t, 3H); MS m/z 477 (M+1).

### Example 66

### 5-[4-(dimethylamino)-1-piperidinyl]-2-({propyl[(8S)-5,6,7,8-tetrahydro-8-quinolinyl]amino}methyl)imidazo[1,2-a]pyridine-3-carbonitrile

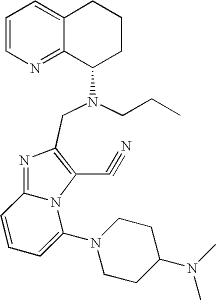

5-[4-(dimethylamino)-1-piperidinyl]-2-({propyl[(8S)-5,6,7,8-tetrahydro-8-quinolinyl]amino}methyl)imidazo[1,2-a]pyridine-3-carbonitrile was prepared from an IBX polystyrene resin oxidation of [5-[4-(dimethylamino)-1-piperidinyl]-2-({propyl[(8S)-5,6,7,8-tetrahydro-8-quinolinyl]amino}methyl)imidazo[1,2-a]pyridin-3-yl]methanol as described herein, then carried forward crude to reaction with hydroxylamine hydrochloride in a similar manner as described herein to obtain a tan solid (47%). ¹H-NMR (CDCl₃): δ 8.43 (d, 1H), 7.42 (m, 1H), 7.31 (m, 2H), 6.99 (dd, 1H), 6.50 (m, 1H), 4.21 (m, 2H), 4.01 (m, 1H), 3.46 (m, 2H), 2.87-2.55 (m, 6H), 2.49 (m, 1H), 2.36 (s, 6H), 2.17 (m, 3H), 1.99 (m, 2H), 1.85 (m, 2H), 1.68 (m, 1H), 1.48 (m, 2H), 0.79 (t, 3H); MS m/z 472 (M+1).

### Example 67

### 5-[4-(dimethylamino)-1-piperidinyl]-2-({propyl[(8S)-5,6,7,8-tetrahydro-8-quinolinyl]amino}methyl)imidazo[1,2-a]pyridine-3-carboxamide

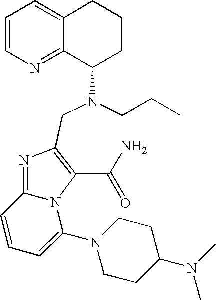

5-[4-(dimethylamino)-1-piperidinyl]-2-({propyl[(8S)-5,6,7,8-tetrahydro-8-quinolinyl]amino}methyl)imidazo[1,2-a]pyridine-3-carboxamide was prepared from 5-[4-(dimethylamino)-1-piperidinyl]-2-({propyl[(8S)-5,6,7,8-tetrahydro-8-quinolinyl]amino}methyl)imidazo[1,2-a]pyridine-3-carbonitrile and sulfuric acid as described herein to give a tan solid (41% yield). ¹H-NMR (CDCl₃): δ 10.36 (s, 1H), 8.31 (d, 1H), 7.21 (m, 3H), 6.96 (dd, 1H), 6.30 (m, 1H), 5.72 (s, 1H), 4.18 (d, 1H), 3.99 (d, 1H), 3.88 (t, 1H), 3.50 (m, 2H), 2.82-2.53 (m, 3H), 2.50-2.26 (m, 9H), 2.21 (m, 1H), 2.02-1.73 (m, 7H), 1.62 (m, 1H), 1.34 (m, 2H), 0.69 (t, 3H); MS m/z 490 (M+1).

### Example 68

### (8S)—N-({5-[4-(dimethylamino)-1-piperidinyl]imidazo[1,2-a]pyridin-2-yl}methyl)-N-(1-methylethyl)-5,6,7,8-tetrahydro-8-quinolinamine (Intermediate)

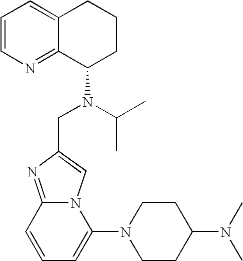

(8S)—N-({5-[4-(dimethylamino)-1-piperidinyl]imidazo[1,2-a]pyridin-2-yl}methyl)-N-(1-methylethyl)-5,6,7,8-tetrahydro-8-quinolinamine was prepared from (8S)—N-({5-[4-(dimethylamino)-1-piperidinyl]imidazo[1,2-a]pyridin-2-yl}methyl)-5,6,7,8-tetrahydro-8-quinolinamine and acetone under reductive amination conditions as described herein to obtain a yellow oil (42% yield). ¹H-NMR (CDCl₃): δ 8.45 (d, 1H), 7.58 (s, 1H), 7.24 (d, 1H), 7.19 (d, 1H), 7.04 (m, 1H), 6.96 (m, 1H), 6.15 (d, 1H), 4.22 (m, 1H), 3.93 (dd, 2H), 3.49 (m, 2H), 3.18 (m, 1H), 2.83-2.73 (m, 1H), 2.71-2.59 (m, 3H), 2.38 (s, 6H), 2.36 (m, 1H), 2.07-1.91 (m, 5H), 1.86-1.71 (m, 2H), 1.64 (m, 1H), 1.12 (d, 6H); MS m/z 447 (M+1).

### Example 69

### [5-[4-(dimethylamino)-1-piperidinyl]-2-({(1-methylethyl)[(8S)-5,6,7,8-tetrahydro-8-quinolinyl]amino}methyl)imidazo[1,2-a]pyridin-3-yl]methanol (Intermediate)

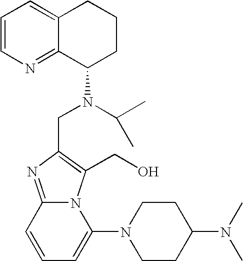

[5-[4-(dimethylamino)-1-piperidinyl]-2-({(1-methylethyl)[(8S)-5,6,7,8-tetrahydro-8-quinolinyl]amino}methyl)imidazo[1,2-a]pyridin-3-yl]methanol was prepared from (8S)—N-({5-[4-(dimethylamino)-1-piperidinyl]imidazo[1,2-a]pyridin-2-yl}methyl)-N-(1-methylethyl)-5,6,7,8-tetrahydro-8-quinolinamine via hydroxymethylation in a similar manner as described herein to give a white solid (56% yield). ¹H-NMR (CDCl₃): δ 8.35 (d, 1H), 7.29 (m, 2H), 7.04 (m, 1H), 6.97 (m, 1H), 6.36 (d, 1H), 5.48 (d, 1H), 5.13 (d, 1H), 4.19 (d, 1H), 4.00 (d, 1H), 3.93 (m, 1H), 3.86 (m, 1H), 3.30 (m, 1H), 2.87-2.71 (m, 3H), 2.63 (m, 1H), 2.57-2.36 (m, 9H), 2.03-1.94 (m, 4H), 1.85 (m, 3H), 1.61 (m, 1H), 1.20 (t, 6H); MS m/z 477 (M+1).

### Example 70

### 5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carbonitrile

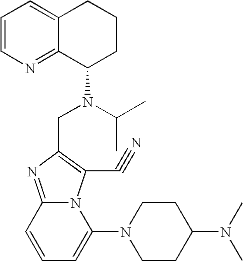

5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carbonitrile was prepared from [5-[4-(dimethylamino)-1-piperidinyl]-2-({(1-methylethyl)[(8S)-5,6,7,8-tetrahydro-8-quinolinyl]amino}methyl)imidazo[1,2-a]pyridin-3-yl]methanol from an IBX polystyrene resin oxidation, then carried forward crude to reaction with hydroxylamine hydrochloride in a similar manner as described herein to obtain a tan solid (47%). ¹H-NMR (CDCl₃): δ 8.38 (d, 1H), 7.37 (m, 1H), 7.25 (m, 2H), 6.93 (dd, 1H), 6.47 (m, 1H), 4.20 (m, 1H), 4.12 (m, 1H), 3.92 (m, 1H), 3.42 (m, 2H), 3.27 (m, 1H), 2.84-2.57 (m, 4H), 2.48 (m, 1H), 2.35 (s, 6H), 2.22-1.99 (m, 5H), 1.83 (m, 2H), 1.67 (m, 1H), 1.15 (m, 6H); MS m/z 472 (M+1).

### Example 71

### 5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carboxamide

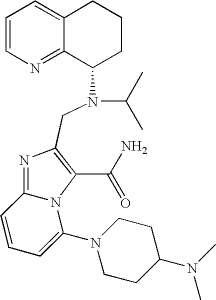

5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carboxamide was prepared from 5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carbonitrile and sulfuric acid as described herein to give a tan solid (50% yield). ¹H-NMR (CDCl₃): δ 10.48 (s, 1H), 8.23 (d, 1H), 7.16 (m, 3H), 6.88 (dd, 1H), 6.26 (m, 1H), 5.80 (s, 1H), 4.22 (d, 4.01 (d, 1H), 3.81 (m, 1H), 3.40 (m, 2H), 2.91 (m, 1H), 2.80-2.50 (m, 5H), 2.38-2.13 (m, 8H), 2.04-1.67 (m, 5H), 1.56 (m, 1H), 1.15 (d, 3H), 0.98 (d, 3H); MS m/z 490 (M+1).

### Example 72

### (8S)—N-{[3-Chloro-5-(4-methyl-1-piperazinyl)imidazo[1,2-a]pyridin-2-yl]methyl}-N-methyl-5,6,7,8-tetrahydro-8-quinolinamine

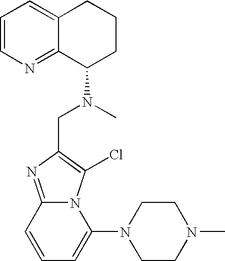

### A) (8S)—N-[(5-Fluoroimidazo[1,2-a]pyridin-2-yl)methyl]-N-{(1S)-1-[4-(methyloxy)phenyl]ethyl}-5,6,7,8-tetrahydro-8-quinolinamine

(8S)—N-[(5-Fluoroimidazo[1,2-a]pyridin-2-yl)methyl]-N-{(1S)-1-[4-(methyloxy)phenyl]ethyl}-5,6,7,8-tetrahydro-8-quinolinamine was prepared from (8S)—N-{(1S)-1-[4-(methyloxy)phenyl]ethyl}-5,6,7,8-tetrahydro-8-quinolinamine and 5-fluoroimidazo[1,2-a]pyridine-2-carbaldehyde via reductive amination in a similar manner as described herein to give a tan solid (98% yield). ¹H-NMR (400 MHz, CDCl₃) δ 8.47 (d, 1H), 7.80 (s, 1H), 7.53 (d, 2H), 7.28-7.24 (m, 1H), 7.20 (d, 1H), 7.09 (m, 1H), 6.96 (dd, 1H), 6.85 (d, 2H), 6.38 (dd, 4.8 Hz, 1H), 4.95 (m, 1H), 4.05-3.95 (m, 2H), 3.78 (s, 4H), 2.67-2.52 (m, 2H), 2.05 (m, 1H), 1.87-1.73 (m, 2H), 1.51 (m, 1H), 1.28 (d, 3H). MS m/z 431 (M+1).

### B) (8S)—N-[(5-Fluoroimidazo[1,2-a]pyridin-2-yl)methyl]-5,6,7,8-tetrahydro-8-quinolinamine

A solution of (8S)—N-[(5-fluoroimidazo[1,2-a]pyridin-2-yl)methyl]-N-{(1S)-1-[4-(methyloxy)phenyl]ethyl}-5,6,7,8-tetrahydro-8-quinolinamine (10.9 g, 25.3 mmol) in 60 mL of 1:1 TFA/dichloromethane was stirred at RT for 3 hours and then concentrated to dryness at reduced pressure. The resulting purple syrup was partitioned between ether and 0.1 N aqueous HCl. The phases were separated and the ether solution washed with two additional portions of 0.1 N aqueous HCl. The combined aqueous solutions were stirred vigorously with addition of 5 N aqueous NaOH until a pH of approximately 12 was attained. The oily mixture was transferred to a separatory funnel and extracted with dichloromethane (4×). The combined extracts were dried over Na₂SO₄ and concentrated to dryness at reduced pressure to afford a yellow-brown syrup. The crude material was purified by flash chromatography (silica gel, 0-10% ammonium hydroxide in acetonitrile) to afford (8S)—N-[(5-fluoroimidazo[1,2-a]pyridin-2-yl)methyl]-5,6,7,8-tetrahydro-8-quinolinamine as a yellow-brown oil in quantitative yield. ¹H-NMR (400 MHz, d₄-MeOH) δ 8.37 (d, 1H), 7.89 (s, 1H), 7.50 (d, 1H), 7.43-7.34 (m, 2H), 7.18 (dd, 1H), 6.67 (m, 1H), 4.14 (d, 1H), 4.06 (d, 1H), 3.88 (m, 1H), 2.94-2.73 (m, 2H), 2.29 (m, 1H), 2.06 (m, 1H), 1.89-1.68 (m, 2H). MS m/z 297 (M+1).

### C) (8S)—N-[(5-Fluoroimidazo[1,2-a]pyridin-2-yl)methyl]-N-methyl-5,6,7,8-tetrahydro-8-quinolinamine

To a stirred solution of (8S)—N-[(5-fluoroimidazo[1,2-a]pyridin-2-yl)methyl]-5,6,7,8-tetrahydro-8-quinolinamine (4.10 g, 13.8 mmol) and glacial acetic acid (2.40 mL, 41.4 mmol) in 60 mL of 1,2-dichloroethane was added 37% aqueous formaldehyde (3.40 mL, 41.4 mmol) followed by NaBH(OAc)₃ (8.80 g, 41.4 mmol). After stirring at RT for 18 hours the solution was diluted with dichloromethane followed by 10% aqueous Na₂CO₃. The resulting bi-phasic mixture was stirred vigorously for 30 minutes and then the phases allowed to separate. The aqueous solution was extracted with 8:2 CHCl₃/iPrOH (3×). The combined organic solutions were dried over Na₂SO₄ and concentrated to dryness at reduced pressure. The crude product was purified by flash chromatography (silica gel, 0-10% ammonium hydroxide in acetonitrile) to afford 3.90 g (91%) of (8S)—N-[(5-fluoroimidazo[1,2-a]pyridin-2-yl)methyl]-N-methyl-5,6,7,8-tetrahydro-8-quinolinamine as a yellow-brown oil. ¹H-NMR (400 MHz, d₄-MeOH) δ 8.41 (d, 1H), 7.98 (s, 1H), 7.50 (d, 1H), 7.38-7.32 (m, 2H), 7.18 (dd, 1H), 6.67 (m, 1H), 4.03 (m, 1H), 3.92-3.80 (m, 2H), 2.88 (m, 1H), 2.74 (m, 1H), 2.32 (s, 3H), 2.18-1.97 (m, 3H), 1.70 (m, 1H). MS m/z 311 (M+1). Alternatively, (8S)—N-[(5-fluoroimidazo[1,2-a]pyridin-2-yl)methyl]-N-methyl-5,6,7,8-tetrahydro-8-quinolinamine can be prepared via the nucleophilic substitution reaction between (8S)—N-methyl-5,6,7,8-tetrahydro-8-quinolinamine and 2-(chloromethyl)-5-fluoroimidazo[1,2-a]pyridine.

### D) (8S)—N-[(3-Chloro-5-fluoroimidazo[1,2-a]pyridin-2-yl)methyl]-N-methyl-5,6,7,8-tetrahydro-8-quinolinamine

A solution of (8S)—N-[(5-fluoroimidazo[1,2-a]pyridin-2-yl)methyl]-N-methyl-5,6,7,8-tetrahydro-8-quinolinamine (1.00 g, 3.22 mmol) and N-chlorosuccinimide (0.640 g, 4.83 mmol) in 25 mL of anhydrous iPrOH was heated to 80° C. with stirring. After 1.5 hours the solution was cooled to RT and concentrated to dryness at reduced pressure. The crude product was subjected to flash chromatography (silica gel, 0-10% ammonium hydroxide in acetonitrile) to afford 0.742 g (67%) of (8S)—N-[(3-chloro-5-fluoroimidazo[1,2-a]pyridin-2-yl)methyl]-N-methyl-5,6,7,8-tetrahydro-8-quinolinamine as a yellow-brown oil. ¹H-NMR (400 MHz, d₄-MeOH) δ 8.37 (d, 1H), 7.50 (d, 1H), 7.38-7.29 (m, 2H), 7.16 (dd, 1H), 6.67 (m, 1H), 3.96 (m, 1H), 3.78 (s, 2H), 2.90 (m, 1H), 2.73 (m, 1H), 2.35 (s, 3H), 2.19-1.95 (m, 3H), 1.71 (m, 1H). MS m/z 345 (M+1).

### E) (8S)—N-{[3-Chloro-5-(4-methyl-1-piperazinyl)imidazo[1,2-a]pyridin-2-yl]methyl}-N-methyl-5,6,7,8-tetrahydro-8-quinolinamine

A solution of (8S)—N-[(3-chloro-5-fluoroimidazo[1,2-a]pyridin-2-yl)methyl]-N-methyl-5,6,7,8-tetrahydro-8-quinolinamine (70 mg, 0.203 mmol) and 1-methylpiperazine (0.113 mL, 1.02 mmol) in 3 mL of DMSO was heated to 80° C. with stirring. After 18 hours the solution was cooled to RT and diluted with EtOAc. The resulting solution was washed with 5% aqueous Na₂CO₃ (3×), saturated aqueous brine (1×), dried over Na₂SO₄ and concentrated to dryness at reduced pressure. The crude product was purified by flash chromatography (silica gel, 0-10% ammonium hydroxide in acetonitrile) to afford 53 mg (62%) of (8S)—N-{[3-chloro-5-(4-methyl-1-piperazinyl)imidazo[1,2-a]pyridin-2-yl]methyl}-N-methyl-5,6,7,8-tetrahydro-8-quinolinamine as a tacky orange foam. ¹H-NMR (400 MHz, d₄-MeOH) δ 8.37 (d, 1H), 7.50 (d, 1H), 7.29-7.24 (m, 2H), 7.17 (dd, 1H), 6.57 (m, 1H), 3.97 (t, 1H), 3.85-3.72 (m, 2H), 3.36-3.25 (m, 2H), 2.98-2.85 (m, 5H), 2.74 (m, 1H), 2.62-2.50 (m, 2H), 2.38 (s, 3H), 2.35 (s, 3H), 2.21-2.07 (m, 2H), 2.02 (m, 1H), 1.73 (m, 1H). MS m/z 425 (M+1).

### Example 73

### (8S)—N-{[3-Chloro-5-(4-ethyl-1-piperazinyl)imidazo[1,2-a]pyridin-2-yl]methyl}-N-methyl-5,6,7,8-tetrahydro-8-quinolinamine

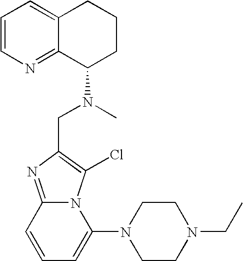

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (0.100 g, 0.290 mmol) was reacted with 1-ethylpiperazine (0.17 g, 1.45 mmol) to afford, following flash chromatography (silica gel, 0-10% ammonium hydroxide in acetonitrile), 99 mg (78%) of (8S)—N-{[3-chloro-5-(4-ethyl-1-piperazinyl)imidazo[1,2-a]pyridin-2-yl]methyl}-N-methyl-5,6,7,8-tetrahydro-8-quinolinamine as a viscous orange oil. ¹H-NMR (400 MHz, d₄-MeOH) δ 8.37 (d, 1H), 7.50 (d, 1H), 7.30-7.23 (m, 2H), 7.18 (dd, 1H), 6.58 (m, 1H), 3.97 (t, 1H), 3.85-3.73 (m, 2H), 3.39-3.28 (m, 2H), 3.06-2.85 (m, 5H), 2.74 (m, 1H), 2.59-2.46 (m, 4H), 2.35 (s, 3H), 2.22-2.07 (m, 2H), 2.20 (m, 1H), 1.72 (m, 1H), 1.17 (t, 3H). MS m/z 439 (M+1).

### Example 74

### (8S)—N-({3-Chloro-5-[(3R)-3-(dimethylamino)-1-pyrrolidinyl]imidazo[1,2-a]pyridin-2-yl}methyl)-N-methyl-5,6,7,8-tetrahydro-8-quinolinamine

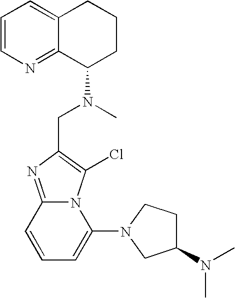

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (0.100 g, 0.290 mmol) was reacted with (3R)—N,N-dimethyl-3-pyrrolidinamine (0.17 g, 1.45 mmol) to afford, following flash chromatography (silica gel, 0-10% ammonium hydroxide in acetonitrile), 76 mg (60%) of (8S)—N-({3-chloro-5-[(3R)-3-(dimethylamino)-1-pyrrolidinyl]imidazo[1,2-a]pyridin-2-yl}methyl)-N-methyl-5,6,7,8-tetrahydro-8-quinolinamine as a viscous orange oil. ¹H-NMR (400 MHz, d₄-MeOH) δ 8.37 (d, 1H), 7.50 (d, 1H), 7.28-7.12 (m, 3H), 6.62 (dd, 1H), 3.96 (t, 1H), 3.78 (s, 2H), 3.51 (m, 1H), 3.36-3.18 (m, 2H), 3.16-2.85 (m, 3H), 3.74 (m, 1H), 2.35 (s, 3H), 2.32 (s, 6H), 2.28-2.06 (m, 3H), 2.05-1.86 (m, 2H), 1.72 (m, 1H). MS m/z 439 (M+1).

### Example 75

### (8S)—N-({3-Chloro-5-[4-(dimethylamino)-1-piperidinyl]imidazo[1,2-a]pyridin-2-yl}methyl)-N-methyl-5,6,7,8-tetrahydro-8-quinolinamine

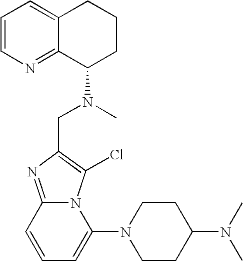

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (0.100 g, 0.290 mmol) was reacted with N,N-dimethyl-4-piperidinamine (0.19 g, 1.45 mmol) to afford, following flash chromatography (silica gel, 0-10% ammonium hydroxide in acetonitrile), 0.103 g (79%) of (8S)—N-({3-chloro-5-[4-(dimethylamino)-1-piperidinyl]imidazo[1,2-a]pyridin-2-yl}methyl)-N-methyl-5,6,7,8-tetrahydro-8-quinolinamine as a viscous orange oil. ¹H-NMR (400 MHz, d₄-MeOH) δ 8.38 (d, 1H), 7.50 (d, 1H), 7.28-7.21 (m, 2H), 7.18 (dd, 1H), 6.53 (dd, 1H), 3.97 (t, 1H), 3.84-3.73 (m, 2H), 3.49-3.39 (m, 2H), 2.92 (m, 1H), 2.79-2.65 (m, 3H), 2.43-2.28 (m, 10H), 2.21-2.08 (m, 2H), 2.06-1.95 (m, 3H), 1.92-1.66 (m, 3H). MS m/z 453 (M+1).

### Example 76

### (8S)—N-{[3-Chloro-5-(4-methylhexahydro-1H-1,4-diazepin-1-yl)imidazo[1,2-a]pyridin-2-yl]methyl}-N-methyl-5,6,7,8-tetrahydro-8-quinolinamine

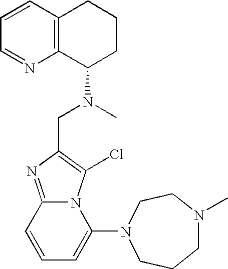

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (80 mg, 0.232 mmol) was reacted with 1-methylhexahydro-1H-1,4-diazepine (0.132 g, 1.16 mmol) to afford, following flash chromatography (silica gel, 0-20% 2M NH₃/MeOH in CH₂Cl₂), 44 mg (43%) of (8S)—N-{[3-chloro-5-(4-methylhexahydro-1H-1,4-diazepin-1-yl)imidazo[1,2-a]pyridin-2-yl]methyl}-N-methyl-5,6,7,8-tetrahydro-8-quinolinamine as a viscous red oil. ¹H-NMR (400 MHz, d₄-MeOH) δ 8.38 (d, 1H), 7.50 (d, 1H), 7.29-7.22 (m, 2H), 7.18 (dd, 1H), 6.69 (t, 1H), 3.97 (t, 1H), 3.83 (m, 2H), 3.57-3.24 (m, 3H), 3.17 (m, 1H), 2.98-2.68 (m, 6H), 2.42 (s, 3H), 2.32 (s, 3H), 2.22-1.91 (m, 5H), 2.72 (m, 1H). MS m/z 439 (M+1).

### Example 77

### N-[3-Chloro-2-({methyl[(8S)-5,6,7,8-tetrahydro-8-quinolinyl]amino}methyl)imidazo[1,2-a]pyridin-5-yl]-N,N′,N′-trimethyl-1,2-ethanediamine

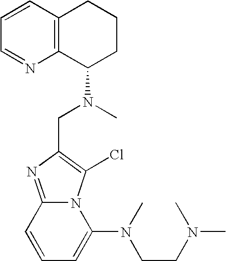

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (80 mg, 0.232 mmol) was reacted with N,N,N′-trimethyl-1,2-ethanediamine (0.119 g, 1.16 mmol) to afford, following flash chromatography (silica gel, 0-20% 2M NH₃/MeOH in CH₂Cl₂), 58 mg (59%) of N-[3-chloro-2-({methyl[(8S)-5,6,7,8-tetrahydro-8-quinolinyl]amino}methyl)imidazo[1,2-a]pyridin-5-yl]-N,N′,N′-trimethyl-1,2-ethanediamine as a viscous orange oil. ¹H-NMR (400 MHz, d₄-MeOH) δ 8.38 (d, 1H), 7.50 (d, 1H), 7.30-7.23 (m, 2H), 7.18 (dd, 1H), 6.62 (dd, 1H), 3.97 (t, 1H), 3.85-3.74 (m, 2H), 3.42-3.29 (m, 1H), 3.13 (m, 1H), 2.92 (m, 1H), 2.82-2.69 (m, 4H), 2.59 (t, 2H), 2.34 (s, 3H), 2.24 (s, 6H), 2.21-2.06 (m, 2H), 2.01 (m, 1H), 1.73 (m, 1H). MS m/z 427 (M+1).

### Example 78

### (8S)—N-({3-Chloro-5-[(3S)-3-methyl-1-piperazinyl]imidazo[1,2-a]pyridin-2-yl}methyl)-N-methyl-5,6,7,8-tetrahydro-8-quinolinamine

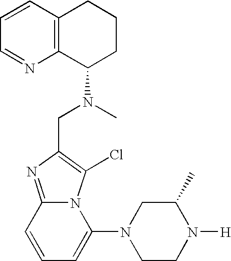

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (80 mg, 0.232 mmol) was reacted with (2S)-2-methylpiperazine (0.116 g, 1.16 mmol) to afford, following flash chromatography (silica gel, 0-20% 2M NH₃/MeOH in CH₂Cl₂), 74 mg (75%) of (8S)—N-({3-chloro-5-[(3S)-3-methyl-1-piperazinyl]imidazo[1,2-a]pyridin-2-yl}methyl)-N-methyl-5,6,7,8-tetrahydro-8-quinolinamine as a viscous oil. ¹H-NMR (400 MHz, d₄-MeOH) δ 8.37 (d, 1H), 7.50 (d, 1H), 7.30-7.22 (m, 2H), 7.17 (dd, 1H), 6.53 (dd, 1H), 3.97 (t, 1H), 3.84-3.72 (m, 2H), 3.34-3.11 (m, 4H), 3.02 (d, 1H), 2.92 (m, 1H), 2.79-2.67 (m, 2H), 2.42-2.31 (m, 4H), 2.20-1.94 (m, 3H), 1.72 (m, 1H), 1.11 (d, 3H). MS m/z 425 (M+1).

### Example 79

### (8S)—N-([3-Chloro-5-[(3S)-3,4-dimethyl-1-piperazinyl]imidazo[1,2-a]pyridin-2-yl]methyl)-N-methyl-5,6,7,8-tetrahydro-8-quinolinamine

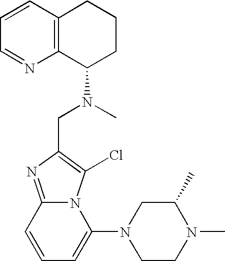

To a stirred solution of (8S)—N-({3-chloro-5-[(3S)-3-methyl-1-piperazinyl]imidazo[1,2-a]pyridin-2-yl}methyl)-N-methyl-5,6,7,8-tetrahydro-8-quinolinamine (61 mg, 0.144 mmol) and glacial acetic acid (25 μL, 0.432 mmol) in 5 mL of 1,2-dichloroethane was added 37% aqueous formaldehyde (35 μL, 0.432 mmol) followed by NaBH(OAc)₃ (92 mg, 0.432 mmol). After stirring at RT for 2 hours the solution was diluted with 5 mL of CH₂Cl₂ followed by 10 mL of 10% aqueous Na₂CO₃ and the resulting bi-phasic mixture was stirred vigorously for 25 minutes. The phases were separated and the aqueous solution extracted with an additional 5 mL portion of CH₂Cl₂. The combined organic solutions were dried over Na₂SO₄ and concentrated to dryness at reduced pressure. The crude product was subjected to flash chromatography (silica gel, 0-20% 2M NH₃/MeOH in CH₂Cl₂) to afford 53 mg (84%) of (8S)—N-({3-chloro-5-[(3S)-3,4-dimethyl-1-piperazinyl]imidazo[1,2-a]pyridin-2-yl}methyl)-N-methyl-5,6,7,8-tetrahydro-8-quinolinamine as a tacky, white foam. ¹H-NMR (400 MHz, d₄-MeOH) δ 8.37 (d, 1H), 7.51 (d, 1H), 7.31-7.22 (m, 2H), 7.18 (dd, 1H), 6.56 (dd, 1H), 3.98 (t, 1H), 3.85-3.72 (m, 2H), 3.36-3.23 (m, 2H), 2.98-2.86 (m, 3H), 2.80-2.63 (m, 2H), 2.61-2.47 (m, 2H), 2.38 (s, 3H), 2.36 (s, 3H), 2.22-1.93 (m, 3H), 1.73 (m, 1H), 1.13 (d, 3H). MS m/z 439 (M+1).

### Example 80

### (8S)—N-({3-Chloro-5-[4-(1-methylethyl)-1-piperazinyl]imidazo[1,2-a]pyridin-2-yl}methyl)-N-methyl-5,6,7,8-tetrahydro-8-quinolinamine

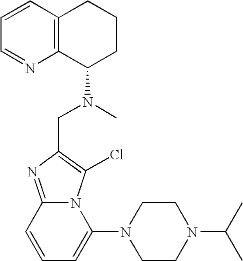

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (80 mg, 0.232 mmol) was reacted with 1-(1-methylethyl)piperazine (0.149 g, 1.16 mmol) to afford, following flash chromatography (silica gel, 0-20% 2M NH₃/MeOH in CH₂Cl₂), 47 mg (45%) of (8S)—N-({3-chloro-5-[4-(1-methylethyl)-1-piperazinyl]imidazo[1,2-a]pyridin-2-yl}methyl)-N-methyl-5,6,7,8-tetrahydro-8-quinolinamine as a viscous orange oil. ¹H-NMR (400 MHz, d₄-MeOH) δ 8.37 (d, 1H), 7.51 (d, 1H), 7.32-7.22 (m, 2H), 7.18 (dd, 1H), 6.57 (d, 1H), 3.98 (t, 1H), 3.84-3.72 (m, 2H), 3.39-3.25 (m, 2H), 3.00-2.83 (m, 5H), 2.81-2.62 (m, 4H), 2.34 (s, 3H), 2.21-2.07 (m, 2H), 2.02 (m, 1H), 1.73 (m, 1H), 1.14 (d, 6H). MS m/z 453 (M+1).

### Example 81

### (8S)—N-({3-Chloro-5-[(8aS)-hexahydropyrrolo[1,2-a]pyrazin-2(1H)-yl]imidazo[1,2-a]pyridin-2-yl}methyl)-N-methyl-5,6,7,8-tetrahydro-8-quinolinamine

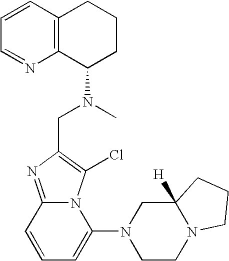

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-methyl-5,6,7,8-tetrahydro-8-quinolinamine (80 mg, 0.232 mmol) was reacted with (8aS)-octahydropyrrolo[1,2-a]pyrazine (0.146 g, 1.16 mmol) to afford, following flash chromatography (silica gel, 0-20% 2M NH₃/MeOH in CH₂Cl₂), 52 mg (50%) of (8S)—N-({3-chloro-5-[(8aS)-hexahydropyrrolo[1,2-a]pyrazin-2(1H)-yl]imidazo[1,2-a]pyridin-2-yl}methyl)-N-methyl-5,6,7,8-tetrahydro-8-quinolinamine as an orange foam. ¹H-NMR (400 MHz, dₐ-MeOH) δ 8.37 (d, 1H), 7.51 (d, 1H), 7.31-7.22 (m, 2H), 7.18 (dd, 1H), 6.59 (dd, 1H), 3.97 (t, 1H), 3.85-3.71 (m, 2H), 3.47 (d, 1H), 3.38-3.27 (m, 1H), 3.19-3.07 (m, 2H), 2.98-2.84 (m, 2H), 2.79-2.57 (m, 3H), 2.50 (m, 1H), 2.39-2.25 (m, 4H), 2.21-2.07 (m, 2H), 2.06-1.79 (m, 4H), 1.73 (m, 1H), 1.48 (m, 1H). MS m/z 451 (M+1).

### Example 82

### (8S)—N-{[3-Chloro-5-(4-methyl-1-piperazinyl)imidazo[1,2-a]pyridin-2-yl]methyl}-N-propyl-5,6,7,8-tetrahydro-8-quinolinamine

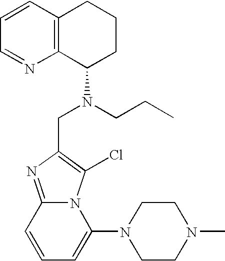

### A) (8S)—N-[(5-Fluoroimidazo[1,2-a]pyridin-2-yl)methyl]-N-propyl-5,6,7,8-tetrahydro-8-quinolinamine

Employing the reductive amination and purification procedure described herein for the preparation of (8S)—N-[(5-fluoroimidazo[1,2-a]pyridin-2-yl)methyl]-N-methyl-5,6,7,8-tetrahydro-8-quinolinamine, (8S)—N-[(5-fluoroimidazo[1,2-a]pyridin-2-yl)methyl]-5,6,7,8-tetrahydro-8-quinolinamine (4.10 g, 13.8 mmol) was reacted with propanal to afford 3.63 g (78%) of (8S)—N-[(5-fluoroimidazo[1,2-a]pyridin-2-yl)methyl]-N-propyl-5,6,7,8-tetrahydro-8-quinolinamine as a yellow-brown oil. ¹H-NMR (400 MHz, d₄-MeOH) δ 8.37 (d, 1H), 7.92 (s, 1H), 7.46 (d, 1H), 7.36-7.26 (m, 2H), 7.12 (dd, 1H), 6.64 (m, 1H), 4.14 (m, 1H), 3.93 (d, 1H), 3.83 (d, 1H), 2.86 (m, 1H), 2.72 (m, 1H), 2.66-2.55 (m, 2H), 2.18-1.91 (m, 3H), 1.69 (m, 1H), 1.57 (m, 2H), 0.81 (t, 3H). MS m/z 339 (M+1).

### B) (8S)—N-[(3-Chloro-5-fluoroimidazo[1,2-a]pyridin-2-yl)methyl]-N-propyl-5,6,7,8-tetrahydro-8-quinolinamine

Employing the reaction and purification protocol described herein for the preparation of (8S)—N-[(3-chloro-5-fluoroimidazo[1,2-a]pyridin-2-yl)methyl]-N-methyl-5,6,7,8-tetrahydro-8-quinolinamine, (8S)—N-[(5-fluoroimidazo[1,2-a]pyridin-2-yl)methyl]-N-propyl-5,6,7,8-tetrahydro-8-quinolinamine (1.00 g, 2.95 mmol) was reacted with N-chlorosuccinimide to afford 0.90 g (82%) of (8S)—N-[(3-chloro-5-fluoroimidazo[1,2-a]pyridin-2-yl)methyl]-N-propyl-5,6,7,8-tetrahydro-8-quinolinamine as a viscous, yellow-brown oil. ¹H-NMR (400 MHz, d₄-MeOH) δ 8.33 (d, 1H), 7.42 (d, 1H), 7.39-7.28 (m, 2H), 7.10 (dd, 1H), 6.64 (m, 1H), 4.14 (t, 1H), 3.95 (d, 1H), 3.83 (d, 1H), 2.86 (m, 1H), 2.76-2.52 (m, 3H), 2.12-1.93 (m, 3H), 1.68 (m, 1H), 1.53-1.39 (m, 2H), 0.77 (t, 31-1). MS m/z 373 (M+1).

### C) (8S)—N-{[3-Chloro-5-(4-methyl-1-piperazinyl)imidazo[1,2-a]pyridin-2-yl]methyl}-N-propyl-5,6,7,8-tetrahydro-8-quinolinamine

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with 1-methylpiperazine (0.108 g, 1.08 mmol) to afford, following flash chromatography (silica gel, 0-20% 2M NH₃/MeOH in CH₂Cl₂), 56 mg (58%) of (8S)—N-{[3-chloro-5-(4-methyl-1-piperazinyl)imidazo[1,2-a]pyridin-2-yl]methyl}-N-propyl-5,6,7,8-tetrahydro-8-quinolinamine as a viscous oil. ¹H-NMR (400 MHz, d₄-MeOH) δ 8.34 (d, 1H), 7.44 (d, 1H), 7.28-7.23 (m, 2H), 7.13 (dd, 1H), 6.57 (m, 1H), 4.17 (t, 1H), 3.98-3.81 (m, 3.38-3.24 (m, 2H), 2.98-2.82 (m, 5H), 2.77-2.50 (m, 5H), 2.39 (s, 3H), 2.13-1.96 (m, 3H), 1.69 (m, 1H), 1.53-1.38 (m, 2H), 0.78 (t, 3H). MS m/z 453 (M+1).

### Example 83

### (8S)—N-{[3-Chloro-5-(4-ethyl-1-piperazinyl)imidazo[1,2-a]pyridin-2-yl]methyl}-N-propyl-5,6,7,8-tetrahydro-8-quinolinamine

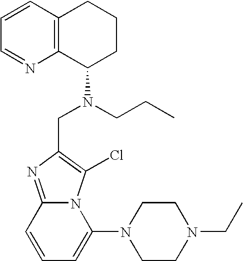

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with 1-ethylpiperazine (0.123 g, 1.08 mmol) to afford, following flash chromatography (silica gel, 0-20% 2M NH₃/MeOH in CH₂Cl₂), 58 mg (58%) of (8S)—N-{[3-chloro-5-(4-ethyl-1-piperazinyl)imidazo[1,2-a]pyridin-2-yl]methyl}-N-propyl-5,6,7,8-tetrahydro-8-quinolinamine as a viscous orange oil. ¹H-NMR (400 MHz, d₄-MeOH) δ 8.32 (d, 1H), 7.45 (d, 1H), 7.29-7.22 (m, 2H), 7.12 (dd, 1H), 6.58 (m, 1H), 4.17 (t, 1H), 3.98-3.80 (m, 2H), 3.40-3.29 (m, 2H), 3.07-2.82 (m, 5H), 2.77-2.45 (m, 7H), 2.12-1.93 (m, 3H), 1.70 (m, 1H), 1.52-1.39 (m, 2H), 1.17 (t, 3H), 0.78 (t, 3H). MS m/z 467 (M+1).

### Example 84

### (8S)—N-{[3-Chloro-5-(4-methylhexahydro-1H-1,4-diazepin-1-yl)imidazo[1,2-a]pyridin-2-yl]methyl}-N-propyl-5,6,7,8-tetrahydro-8-quinolinamine

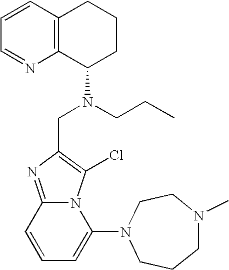

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with 1-methylhexahydro-1H-1,4-diazepine (0.123 g, 1.08 mmol) to afford, following flash chromatography (silica gel, 0-20% 2M NH₃/MeOH in CH₂Cl₂), 34 mg (34%) of (8S)—N-{[3-chloro-5-(4-methylhexahydro-1H-1,4-diazepin-1-yl)imidazo[1,2-a]pyridin-2-yl]methyl}-N-propyl-5,6,7,8-tetrahydro-8-quinolinamine as a viscous orange oil. ¹H-NMR (400 MHz, d₄-MeOH) δ 8.33 (d, 1H), 7.44 (d, 1H), 7.28-7.23 (m, 2H), 7.11 (dd, 1H), 6.69 (m, 1H), 4.16 (t, 1H), 3.98-3.79 (m, 2H), 3.58-3.29 (m, 3H), 3.18 (m, 1H), 2.93-2.77 (m, 5H), 2.75-2.65 (m, 3H), 2.45 (s, 3H), 2.13-1.92 (m, 5H), 1.68 (m, 1H), 1.51-1.39 (m, 2H), 0.77 (t, 3H). MS m/z 467 (M+1).

### Example 85

### (8S)—N-({3-Chloro-5-[4-(1-methylethyl)-1-piperazinyl]imidazo[1,2-a]pyridin-2-yl}methyl)-N-propyl-5,6,7,8-tetrahydro-8-quinolinamine

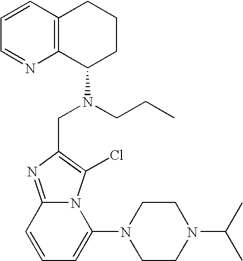

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with 1-(1-methylethyl)piperazine (0.138 g, 1.08 mmol) to afford, following flash chromatography (silica gel, 0-20% 2M NH₃/MeOH in CH₂Cl₂), 71 mg (69%) of (8S)—N-({3-chloro-5-[4-(1-methylethyl)-1-piperazinyl]imidazo[1,2-a]pyridin-2-yl}methyl)-N-propyl-5,6,7,8-tetrahydro-8-quinolinamine as a viscous orange oil. ¹H-NMR (400 MHz, d₄-MeOH) δ 8.33 (d, 1H), 7.45 (d, 1H), 7.28-7.23 (m, 2H), 7.12 (dd, 1H), 6.57 (m, 1H), 4.17 (t, 1H), 3.98-3.80 (m, 2H), 3.38-3.26 (m, 2H), 3.02-2.82 (m, 4H), 2.80-2.53 (m, 7H), 2.13-1.94 (m, 3H), 1.69 (m, 1H), 1.51-1.39 (m, 2H), 1.16 (d, 6H), 0.77 (t, 3H). MS m/z 481 (M+1).

### Example 86

### (8S)—N-({3-Chloro-5-[(3R)-3-(dimethylamino)-1-pyrrolidinyl]imidazo[1,2-a]pyridin-2-yl}methyl)-N-propyl-5,6,7,8-tetrahydro-8-quinolinamine

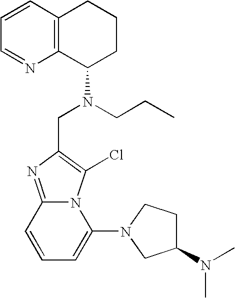

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with (3R)—N,N-dimethyl-3-pyrrolidinamine (0.123 g, 1.08 mmol) to afford, following flash chromatography (silica gel, 0-20% 2M NH₃/MeOH in CH₂Cl₂), 58 mg (58%) of (8S)—N-({3-chloro-5-[(3R)-3-(dimethylamino)-1-pyrrolidinyl]imidazo[1,2-a]pyridin-2-yl}methyl)-N-propyl-5,6,7,8-tetrahydro-8-quinolinamine as a viscous orange oil. ¹H-NMR (400 MHz, d₄-MeOH) δ 8.34 (d, 1H), 7.46 (d, 1H), 7.26-7.18 (m, 2H), 7.12 (m, 1H), 6.62 (dd, 1H), 4.17 (t, 1H), 3.97-3.79 (m, 2H), 3.50 (m, 1H), 3.38-3.17 (m, 2H), 3.17-2.93 (m, 2H), 2.87 (m, 1H), 2.77-2.58 (m, 3H), 2.32 (s, 6H), 2.26 (m, 1H), 2.12-1.87 (m, 4H), 1.69 (m, 1H), 1.52-1.39 (m, 2H), 0.77 (t, 3H). MS m/z 467 (M+1).

### Example 87

### (8S)—N-({3-Chloro-5-[4-(dimethylamino)-1-piperidinyl]imidazo[1,2-a]pyridin-2-yl}methyl)-N-propyl-5,6,7,8-tetrahydro-8-quinolinamine

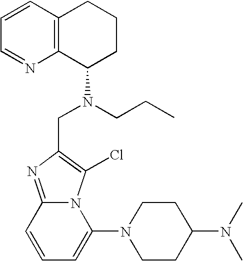

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with N,N-dimethyl-4-piperidinamine (0.138 g, 1.08 mmol) to afford, following flash chromatography (silica gel, 0-20% 2M NH₃/MeOH in CH₂Cl₂), 64 mg (62%) of (8S)—N-({3-chloro-5-[4-(dimethylamino)-1-piperidinyl]imidazo[1,2-a]pyridin-2-yl}methyl)-N-propyl-5,6,7,8-tetrahydro-8-quinolinamine as a viscous oil. ¹H-NMR (400 MHz, d₄-MeOH) δ 8.33 (d, 1H), 7.45 (d, 1H), 7.27-7.20 (m, 2H), 7.12 (dd, 1H), 6.53 (m, 1H), 4.17 (t, 1H), 3.98-3.80 (m, 2H), 3.51-3.38 (m, 2H), 2.87 (m, 1H), 2.78 (m, 5H), 2.44-2.31 (m, 7H), 2.12-1.95 (m, 5H), 1.93-1.78 (m, 2H), 1.68 (m, 1H), 1.50-1.39 (m, 2H), 0.77 (t, 3H). MS m/z 481 (M+1).

### Example 88

### (8S)—N-({3-Chloro-5-[(3S)-3-methyl-1-piperazinyl]imidazo[1,2-a]pyridin-2-yl}methyl)-N-propyl-5,6,7,8-tetrahydro-8-quinolinamine

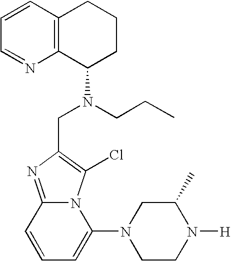

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with (2S)-2-methylpiperazine (0.108 g, 1.08 mmol) to afford, following flash chromatography (silica gel, 0-20% 2M NH₃/MeOH in CH₂Cl₂), 58 mg (60%) of (8S)—N-({3-chloro-5-[(3S)-3-methyl-1-piperazinyl]imidazo[1,2-a]pyridin-2-yl}methyl)-N-propyl-5,6,7,8-tetrahydro-8-quinolinamine as a viscous oil. ¹H-NMR (400 MHz, d₄-MeOH) δ 8.33 (d, 1H), 7.44 (d, 1H), 7.27-7.21 (m, 2H), 7.12 (dd, 1H), 6.53 (m, 1H), 4.17 (t, 1H), 3.97-3.79 (m, 2H), 3.31-3.12 (m, 4H), 3.03 (m, 1H), 2.87 (m, 1H), 2.78-1.57 (m, 4H), 2.38 (t, 1H), 2.12-1.94 (m, 3H), 1.69 (m, 1H), 1.51-1.38 (m, 2H), 1.12 (d, 3H), 0.77 (t, 3H). MS m/z 453 (M+1).

### Example 89

### N-[3-Chloro-2-({propyl[(8S)-5,6,7,8-tetrahydro-8-Quinolinyl]amino}methyl)imidazo[1,2-a]pyridin-5-yl]-N,N′,N′-trimethyl-1,2-ethanediamine

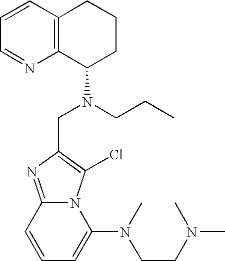

According to the procedure described herein for the preparation of (8S)—N-{[3-chloro-5-(4-methyl-1-piperazinyl)imidazo[1,2-a]pyridin-2-yl]methyl}-N-methyl-5,6,7,8-tetrahydro-8-quinolinamine, (8S)—N-[(3-chloro-5-fluoroimidazo[1,2-a]pyridin-2-yl)methyl]-N-propyl-5,6,7,8-tetrahydro-8-quinolinamine (80 mg, 0.215 mmol) was reacted with N,N,N′-trimethyl-1,2-ethanediamine (0.110 g, 1.08 mmol) to afford, following flash chromatography (silica gel, 0-20% 2M NH₃/MeOH in CH₂Cl₂), 72 mg (73%) of N-[3-chloro-2-({propyl[(8S)-5,6,7,8-tetrahydro-8-quinolinyl]amino}methyl)imidazo[1,2-a]pyridin-5-yl]-N,N′,N′-trimethyl-1,2-ethanediamine as a viscous oil. ¹H-NMR (400 MHz, d₄-MeOH) δ 8.34 (d, 1H), 7.46 (d, 1H), 7.28-7.22 (m, 2H), 7.12 (dd, 1H), 6.62 (m, 1H), 4.18 (m, 1H), 3.95 (d, 1H), 3.84 (m, 1H), 3.35 (m, 1H), 3.13 (m, 1H), 2.87 (m, 1H), 2.82-2.77 (m, 3H), 2.76-2.53 (m, 5H), 2.25 (s, 6H), 2.12-1.93 (m, 3H), 1.69 (m, 1H), 1.50-1.37 (m, 2H), 0.77 (t, 3H). MS m/z 455 (M+1).

### Example 90

### (8S)—N-{[8-Fluoro-5-(4-methyl-1-piperazinyl)imidazo[1,2-a]pyridin-2-yl]methyl}-N-propyl-5,6,7,8-tetrahydro-8-quinolinamine

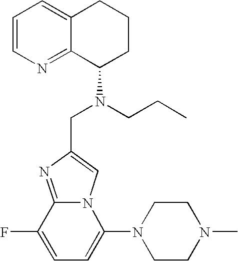

### A) 3,6-Difluoro-2-pyridinamine

A mixture of 2,3,6-trifluoropyridine (8.18 g, 61.5 mmol) and concentrated NH₄OH (40 mL) was heated at 100° in a steel bomb for 3 h. The vessel was chilled in an ice bath and the resulting precipitate collected by filtration. After washing with cold water, the solid was dried to afford 3,6-difluoro-2-pyridinamine (5.95 g, 74%) as fine white needles. ¹H NMR (CDCl₃): 8.7.25 (m, 1H), 6.18 (d, 1H). MS m/z 131 (M+1).

### B) 2-(Dichloromethyl)-5,8-difluoroimidazo[1,2-a]pyridine

A solution of 3,6-difluoro-2-pyridinamine (4.19 g, 32.2 mmol) in 1,4-dioxane (60 mL) was treated with 1,1,3-trichloroacetone (25 g, 155 mmol) and heated in a 100° bath for 4 days. After cooling to 50°, another 25 g portion of 1,1,3-trichloroacetone was added and the reaction was reheated to 100° for an additional 1 day. The reaction was cooled to it and concentrated. The residue was filtered through a pad of silica gel (gradient elution of 0 to 50% ethyl acetate in hexanes). A final purification by flash chromatography (silica gel, 0 to 40% ethyl acetate in hexanes) afforded 3.98 g (52%) of 2-(dichloromethyl)-5,8-difluoroimidazo[1,2-a]pyridine as a gold crystalline solid. ¹H NMR (DMSO-d₆): δ 8.24 (d, 1H), 7.60 (s, 1H), 7.35 (m, 1H), 6.83 (m, 1H). MS m/z 237 (M+1).

### C) 5,8-Difluoroimidazo[1,2-a]pyridine-2-carbaldehyde

2-(dichloromethyl)-5,8-difluoroimidazo[1,2-a]pyridine (3.95 g, 16.7 mmol) and sodium acetate (3.42 g, 41.7 mmol) were combined with ethanol (50 mL) and water (100 mL) and heated at 70° for 2 h. The reaction was concentrated to remove the ethanol, then extracted with dichloromethane (3×). The combined organic layers were washed with brine, dried (Na₂SO₄) and concentrated to afford 2.75 g (91%) of 5,8-difluoroimidazo[1,2-a]pyridine-2-carbaldehyde as a light brown solid. ¹H NMR (CDCl₃): δ 10.21 (s, 1H), 8.23 (s, 1H), 7.03 (m, 1H), 6.52 (m, 1H). MS m/z 183 (M+1).

### D) (8S)—N-{(1S)-1-[4-(Methyloxy)phenyl]ethyl}-5,6,7,8-tetrahydro-8-quinolinamine

A solution of (S)-(−)-1-(4-methoxyphenyl)ethylamine (25.0 g, 166 mmol) and 6,7-dihydro-8(5H)-quinolinone (24.0 g, 166 mmol, J. Org. Chem., 2002, 67, 2197-2205) in dichloromethane was treated with glacial acetic acid (14.0 mL, 249 mmol) and sodium triacetoxyborohydride (53.0 g, 249 mmol). The reaction mixture was stirred at room temperature for 15 hours and then treated with sodium carbonate (106 g, 996 mmol) dissolved in water. The resulting mixture was stirred for 30 minutes and then diluted with dichloromethane. The phases were separated and the aqueous solution extracted with an additional portion of dichloromethane. The combined organic solutions were dried over MgSO₄ and concentrated to dryness at reduced pressure. The crude product was purified by flash chromatography (silica gel, gradient elution of dichloromethane to 97:3 dichloromethane/2M ammonia in MeOH) followed by recrystallization from hexane to afford 33 g (70%) of (8S)—N-{(1S)-1-[4-(methyloxy)phenyl]ethyl}-5,6,7,8-tetrahydro-8-quinolinamine as a white crystalline solid. ¹H NMR (CDCl₃): δ 8.40 (m, 1H), 7.33 (m, 3H), 7.04 (m, 1H), 6.84 (d, 2H), 4.02 (m, 1H), 3.83-3.78 (m, 4H), 2.73-2.62 (m, 2H), 1.82 (m, 1H), 1.72 (m, 1H), 1.57 (m, 2H), 1.43 (d, 3H).

### E) 8S-N-{(1S)-1-[4-(Methyloxy)phenyl]ethyl}-N-propyl-5,6,7,8-tetrahydro-8-quinolinamine

To a stirred mixture of (8S)—N-{(1S)-1-[4-(methyloxy)phenyl]ethyl}-5,6,7,8-tetrahydro-8-quinolinamine (10.46 g, 37.0 mmol), propionaldehyde (4.0 mL, 55.6 mmol), and glacial acetic acid (3.2 mL, 55.6 mmol) in 100 mL of 1,2-dichloroethane was added NaBH(OAc)₃ (11.78 g, 55.6 mmol). After stirring at RT for 4 hours the mixture was treated with 10% aqueous Na₂CO₃ and stirred overnight. The mixture was extracted with dichloromethane (3×). The combined organic solutions were washed with saturated aqueous brine, dried over Na₂SO₄, and concentrated to dryness at reduced pressure to afford (8S)—N-{(1S)-1-[4-(methyloxy)phenyl]ethyl}-N-propyl-5,6,7,8-tetrahydro-8-quinolinamine in quantitative yield as a yellow oil. ¹H NMR (CDCl₃): 8.41 (d, 1H), 7.41 (d, 2H), 7.23 (m, 1H), 6.99 (m, 1H), 6.81 (d, 2H), 4.58 (q, 1H), 3.95 (t, 1H), 3.78 (s, 3H), 2.79 (m, 1H), 2.61 (m, 1H), 2.45-2.23 (m, 2H), 2.01-1.83 (m, 3H), 1.57 (m, 1H), 1.37 (d, 3H), 1.20-1.01 (m, 2H), 1.58 (t, 3H). MS m/z 325 (M+H).

### F) (8S)—N-Propyl-5,6,7,8-tetrahydro-8-quinolinamine

A solution of (8S)—N-{(1S)-1-[4-(methyloxy)phenyl]ethyl}-N-propyl-5,6,7,8-tetrahydro-8-quinolinamine (12.02 g, 37.0 mmol) in 45 mL of 1:2 trifluoroacetic acid/dichloromethane was stirred at RT for 2 h and then concentrated to dryness by rotary evaporation. The residue was evaporated with dichloromethane (3×), then taken up in dichloromethane, washed with 10% aqueous Na₂CO₃, then brine. After drying over Na₂SO₄ and concentrating, the residue was purified by flash chromatography (silica gel, 0 to 10% methanol in dichloromethane) to afford 4.40 g (62%) of (8S)—N-propyll -5,6,7,8-tetrahydro-8-quinolinamine as a gold oil. ¹H NMR (CDCl₃): δ 8.40 (d, 1H), 7.38 (d, 1H), 7.05 (m, 1H), 3.80 (t, 1H), 2.82-2.60 (m, 4H), 2.18 (m, 1H), 2.02 (m, 1H), 1.75 (m, 2H), 1.61 (m, 2H), 0.99 (t, 3H).

### G) (8S)—N-[(5,8-Difluoroimidazo[1,2-a]pyridin-2-yl)methyl]-N-propyl-5,6,7,8-tetrahydro-8-quinolinamine

A solution of 5,8-difluoroimidazo[1,2-a]pyridine-2-carbaldehyde (1.55 g, 8.51 mmol) and (8S)—N-propyll -5,6,7,8-tetrahydro-8-quinolinamine (1.62 g, 8.51 mmol) in 1,2-dichloroethane (100 mL) was treated with glacial acetic acid (0.73 mL, 12.8 mmol). To this was added NaBH(OAc)₃ (2.71 g, 12.8 mmol) in portions over 20 min. After stirring overnight, the reaction was treated with 10% aqueous Na₂CO₃ and stirred 1 h. The mixture was extracted with dichloromethane (2×). The combined organic layers were washed with brine, dried over Na₂SO₄ and concentrated. Flash chromatography (silica gel, 0 to 5% NH₄OH in acetonitrile) afforded 1.94 g (96%) of (8S)—N-[(5,8-difluoroimidazo[1,2-a]pyridin-2-yl)methyl]-N-propyl-5,6,7,8-tetrahydro-8-quinolinamine as a brown oil. ¹H NMR (DMSO-d₆): δ 8.39 (d, 1H), 7.84 (m, 1H), 7.41 (d, 1H), 7.21-7.05 (m, 2H), 6.70 (m, 1H), 4.13-3.98 (m, 3H), 2.80-2.60 (m, 3H), 2.45 (m, 1H), 2.00-1.77 (m, 3H), 1.61 (m, 1H), 1.36 (m, 2H), 0.78 (t, 3H). MS m/z 357 (M+1).

### H) (8S)—N-{[8-Fluoro-5-(4-methyl-1-piperazinyl)imidazo[1,2-a]pyridin-2-yl]methyl}-N-propyl-5,6,7,8-tetrahydro-8-quinolinamine

A solution of (8S)—N-[(5,8-difluoroimidazo[1,2-a]pyridin-2-yl)methyl]-N-propyl-5,6,7,8-tetrahydro-8-quinolinamine (73 mg), 0.22 mmol) in DMSO (0.5 mL) was treated with N-methylpiperazine (0.11 mL, 1.01 mmol) and heated to 70° for 20 h. After cooling to rt, the mixture was poured into a solution of 10% aqueous Na₂CO₃ and brine and extracted with ethyl acetate (2×). The combined organic layers were dried over Na₂SO₄ and concentrated. Purification by flash chromatography (silica gel, 0 to 10% NH₄OH in acetonitrile) afforded 62 mg (70%) of (8S)—N-{[8-fluoro-5-(4-methyl-1-piperazinyl)imidazo[1,2-a]pyridin-2-yl]methyl}-N-propyl-5,6,7,8-tetrahydro-8-quinolinamine as a white crystalline solid. ¹H NMR (DMSO-d₆): δ 8.39 (d, 1H), 7.74 (d, 1H), 7.43 (m, 1H), 7.13-7.02 (m, 2H), 6.28 (m, 1H), 4.05-3.76 (m, 3H), 3.01 (broad s, 4H), 2.77-2.50 (m, 8H), 2.26 (s, 3H), 1.96-1.79 (m, 3H), 1.59 (m, 1H), 1.34 (m, 2H), 0.78 (t, 3H). MS m/z 437 (M+1).

### Example 91

### (8S)—N-{[3-Chloro-8-fluoro-5-(4-methyl-1-piperazinyl)imidazo[1,2-a]pyridin-2-yl]methyl}-N-propyl-5,6,7,8-tetrahydro-8-quinolinamine

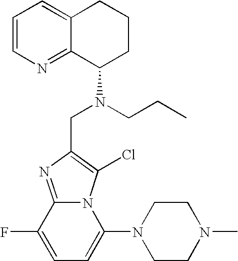

A solution of (8S)—N-{[8-fluoro-5-(4-methyl-1-piperazinyl)imidazo[1,2-a]pyridin-2-yl]methyl}-N-propyl-5,6,7,8-tetrahydro-8-quinolinamine (124 mg, 0.28 mmol, prepared as in Example 90) in isopropanol (10 mL) was treated with N-chlorosuccinimide (57 mg, 0.43 mmol) and heated to reflux for 1.5 h. After cooling to it the reaction was concentrated. The residue was purified by flash chromatography (silica gel, 0 to 5% NH₄OH in acetonitrile) to afford 90 mg (67%) of (8S)—N-{[3-chloro-8-fluoro-5-(4-methyl-1-piperazinyl)imidazo[1,2-a]pyridin-2-yl]methyl}-N-propyl-5,6,7,8-tetrahydro-8-quinolinamine as a yellow oil. ¹H NMR (DMSO-d₅): δ 8.36 (d, 1H), 7.42 (d, 1H), 7.08 (m, 2H), 6.46 (m, 1H), 4.07-3.75 (m, 3H), 3.11 (m, 2H), 2.80-2.61 (m, 8H), 2.31-2.05 (m, 5H), 1.88 (m, 3H), 1.59 (m, 1H), 1.29 (m, 2H), 0.67 (t, 3H). MS m/z 471 (M+1).

### Biological Section

### HOS HIV-1 Infectivity Assay

### HIV Virus Preparation

Compounds were profiled against two HIV-1 viruses, the M-tropic (CCR5 utilizing) Ba-L strain and the T-tropic (CXCR4 utilizing) IIIB strain. Ba-L was propagated in either peripheral blood lymphocytes or SupT1/CCR5+/CXCR4+ cells. IIIB was propagated in peripheral blood lymphocytes. Compounds were tested for their ability to block infection of the HOS cell line (expressing hCXCR4/hCCR5/hCD4/pHIV-LTR-luciferase) by either HIV-1 Ba-L or HIV-1 IIIB. Compound cytotoxicity was also examined in the absence of virus addition.

### HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS to a concentration of 120,000 cells/ml. The cells were plated into 96-well plates (50 μl per well) and the plates were placed in a tissue culture incubator (370° C.; 5% CO₂195% air) for a period of 24 h.

Subsequently, 50 μl of the desired drug solution (2 times the final concentration) was added to each well and the plates were returned to the tissue culture incubator (370° C.; 5% CO₂/95% air) for 1 h. Following this incubation, 60 μl of diluted virus (4 times the final concentration) was added to 60 μl of 2× the final desired concentration of the compound and 100 μl of this compound/virus mix was added to each well (approximately 2 million RLU per well of virus). The plates were returned to the tissue culture incubator (370° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

TABLE 1

| Example | Structure | Activity Level* |
|---|---|---|
| 14 | 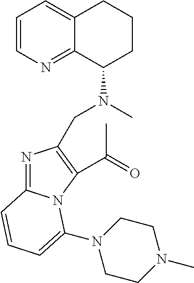 | A |
| 16 | 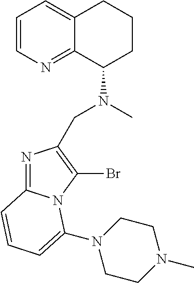 | A |
| 17 | 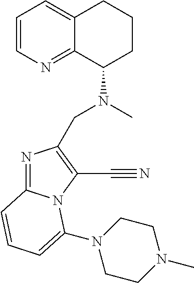 | A |
| 18 | 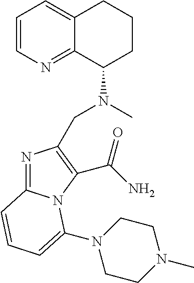 | A |
| 21 | 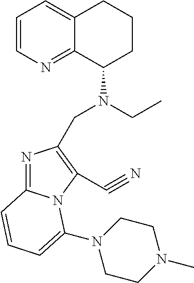 | A |
| 22 | 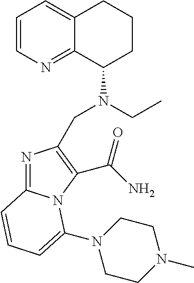 | A |
| 25 | 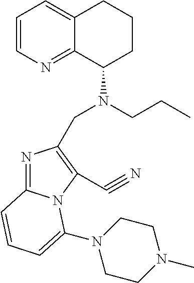 | A |
| 26 | 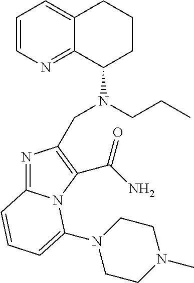 | A |
| 29 | 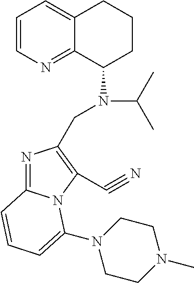 | A |
| 30 | 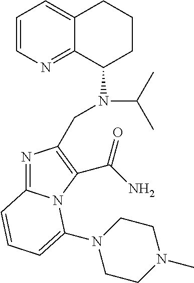 | A |
| 33 | 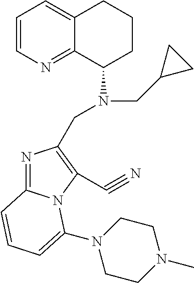 | A |
| 38 | 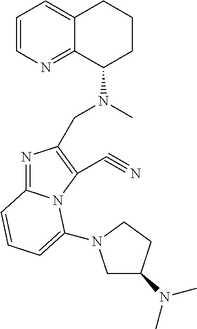 | A |
| 39 | 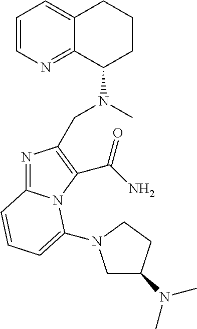 | A |
| 42 | 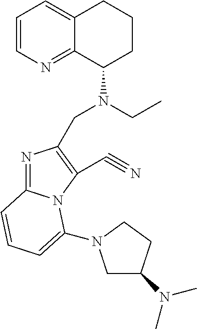 | A |
| 43 | 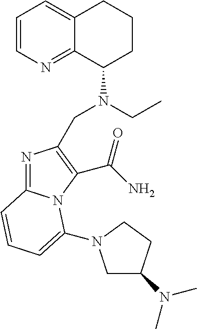 | A |
| 46 | 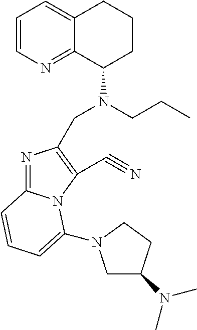 | A |
| 47 | 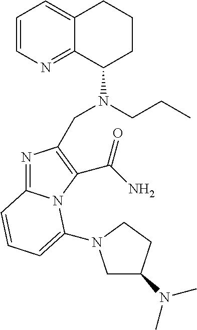 | A |
| 50 | 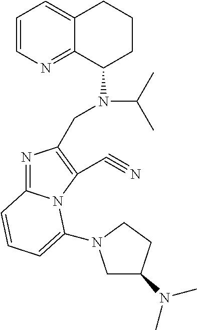 | A |
| 51 | 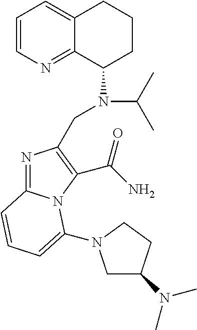 | A |
| 53 | 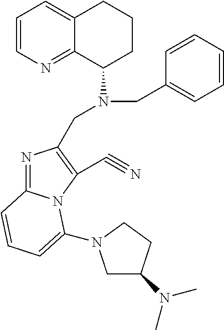 | A |
| 58 | 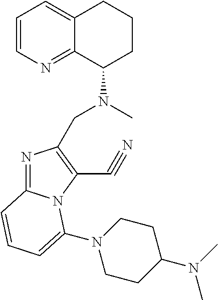 | A |
| 59 | 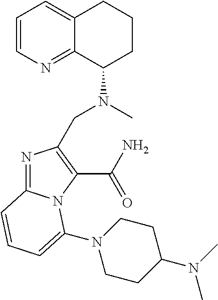 | A |
| 62 | 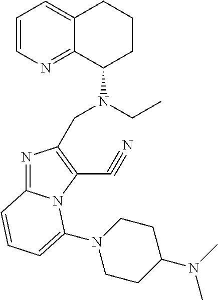 | A |
| 63 | 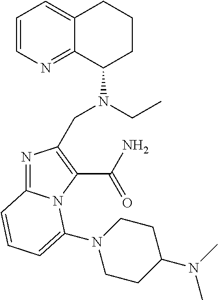 | A |
| 64 | 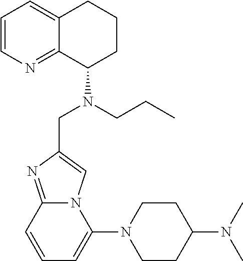 | A |
| 66 | 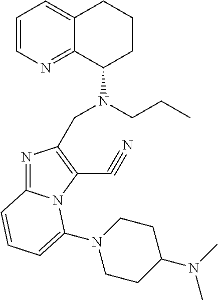 | A |
| 67 | 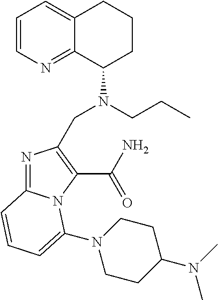 | A |
| 70 | 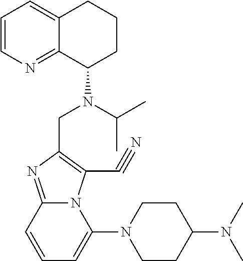 | A |
| 71 | 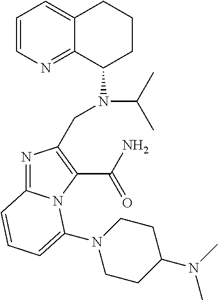 | A |
| 72 | 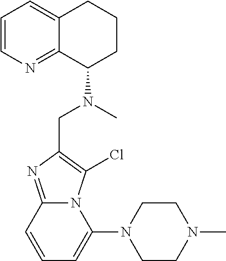 | A |
| 73 | 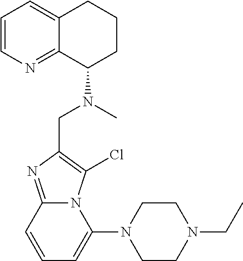 | A |
| 74 | 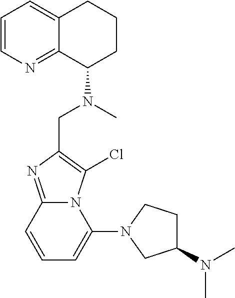 | A |
| 75 | 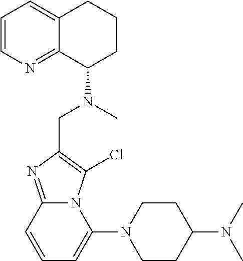 | A |
| 76 | 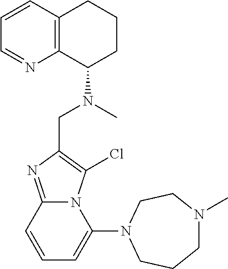 | A |
| 77 | 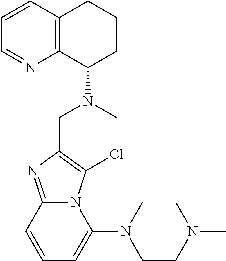 | A |
| 78 | 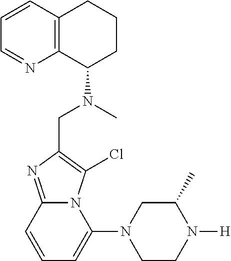 | A |
| 79 | 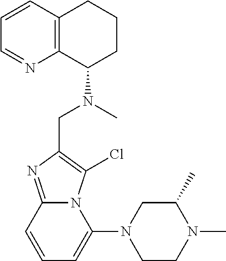 | A |
| 80 | 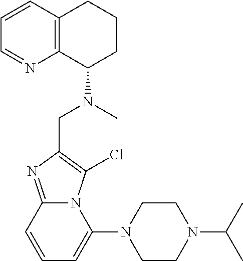 | A |
| 81 | 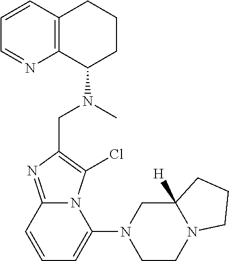 | A |
| 82 | 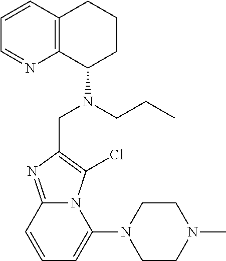 | A |
| 83 | 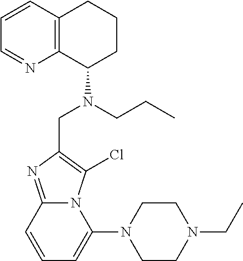 | A |
| 84 | 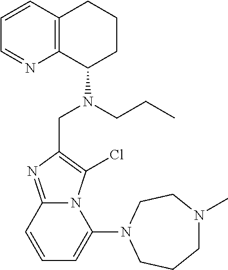 | A |
| 85 | 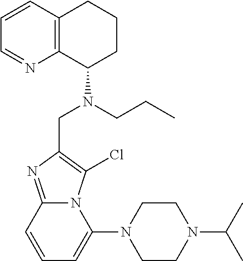 | A |
| 86 | 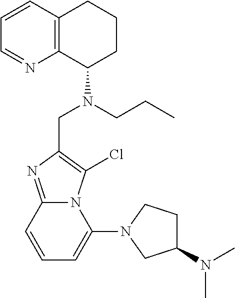 | A |
| 87 | 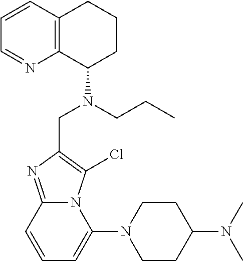 | A |
| 88 | 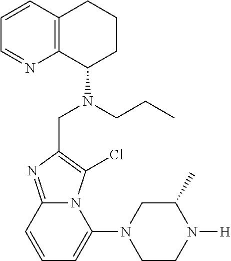 | A |
| 89 | 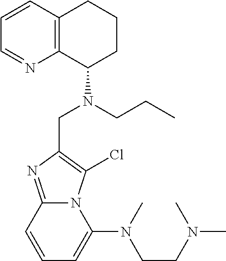 | A |
| 90 | 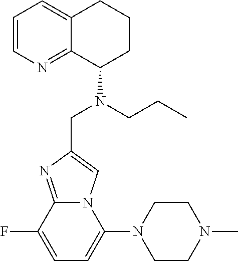 | A |

*“A” indicates an activity level of less than 100 nM in the HIV infectivity assay.
“B” indicates an activity level of between 100 nM to 500 nM in the HIV infectivity assay.
“C” indicates an activity level of between 500 nM and 10 μM in the HIV infectivity assay.

Compounds of the present invention demonstrate anti-HIV activity in the range of IC₅₀ of about 1 nM to about 50 μM. In one aspect of the invention, compounds of the present invention have anti-HIV activity in the range of up to about 100 nM. In another aspect of the invention, compounds of the present invention have anti-HIV activity in the range of from about 100 nM to about 500 nM. In another aspect of the invention, compounds of the present invention have anti-HIV activity in the range of from about 500 nM to 10 μM. In another aspect of the invention, compounds have anti-HIV activity in the range of from about 10 μM to about 50 μM. Moreover, compounds of the present invention are believed to provide a desired pharamcokinetic profile. Also, compounds of the present invention are believed to provide a desired selectivity, such as specificity between toxicity and activity.

Test compounds were employed in free or salt form.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I): 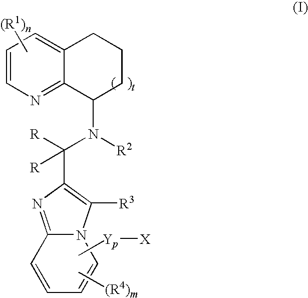
wherein:
t is 0, 1, or 2;
each R independently is H, C₁-C₈ alkyl, C₂-C₆ alkenyl, C₂-C₆ alkynyl, C₁-C₈ haloalkyl, C₃-C₈ cycloalkyl, —R^{a}Ay, —R^{a}OR¹⁰, —R^{a}N(R¹⁰)₂ or —R^{a}S(O)_{q}R¹⁰;
each R¹ independently is halogen, C₁₋₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —N(H)Ay, -Het, —N(H)Het, —OR¹⁰, —OAy, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, —S(O)_{q}Ay, cyano, nitro, or azido;
n is 0, 1, or 2;
m is 0, 1, or 2;
R² is H, C₁-C₈ alkyl, C₂-C₆ alkenyl, C₂-C₆ alkynyl, C₁-C₈ haloalkyl, C₃-C₈ cycloalkyl, —R^{a}cycloalkyl, —R^{a}Ay, —R^{a}OR⁵, or —R^{a}S(O)_{q}R⁵, wherein R² is not amine or alkylamine, or substituted with amine or alkylamine;
R³ is halogen, —N(R¹¹)R¹², OR¹¹, —C(O)R¹¹, —C(O)N(R¹¹)R¹², —N(R¹¹)C(O)R¹², —N(R¹¹)C(O)N(R¹¹)R¹², —N(R¹¹)C(O)OR¹², —S(O)₂N(R¹¹)R¹², —N(R¹¹)S(O)₂R¹², cyano, nitro, or azido;
each R⁴ independently is halogen, C₁-C₈ haloalkyl, C₁-C₈ alkyl, C₂-C₆ alkenyl, C₂-C₆ alkynyl, C₃-C₈ cycloalkyl, C₃-C₈cycloalkenyl, -Ay, —N(H)Ay, -Het, —N(H)Het, —OR¹⁰, —OAy, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁶, —R^{a}CO₂R¹⁰, —C(O)N(R⁶)R⁷, —C(O)Ay, —C(O)Het, —S(O)₂N(R⁶)R⁷, —S(O)_{q}R¹⁰, —S(O)_{q}Ay, —S(O)_{q}Het, cyano, nitro, or azido;
each R⁵ independently is H, C₁-C₈ alkyl, C₂-C₆ alkenyl, C₂-C₆ alkynyl, C₃-C₈ cycloalkyl, or -Ay;
p is 0 or 1;
Y is —NR¹⁰—, —O—, —C(O)NR¹⁰—, —N(R¹⁰)C(O)—, —C(O)—, —C(O)O—, —N(R¹⁰)C(O)N(R¹⁰)—, —S(O)_{q}—, —S(O)_{q}N(R¹⁰)—, or —N(R¹⁰)S(O)_{q}—;
X is —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₈ cycloalkylene, C₂-C₆ alkenylene, C₃-C₈ cycloalkenylene, or C₂-C₆ alkynylene, and is optionally substituted with one or more C₁-C₈ alkyl, hydroxyl or oxo;
each R¹⁰ independently is H, C₁-C₈ alkyl, C₃-C₈ cycloalkyl, C₂-C₆ alkenyl, C₂-C₆ alkynyl, C₃-C₈ cycloalkenyl, —R^{a}cycloalkyl, —R^{a}OR⁵, R^{a}N(R⁶)R⁷ or R^{a}Het.
each R¹¹ and R¹² independently is H, C₁-C₈ alkyl, C₃-C₈ cycloalkyl, C₂-C₆ alkenyl, C₂-C₆ alkynyl, C₃-C₈ cycloalkenyl, —R^{a}cycloalkyl, —R^{a}OR⁵,
each of R⁶ and R⁷ independently are selected from H, C₁-C₈ alkyl, C₁-C₈ alkenyl, C₂-C₆ alkynyl, C₃-C₈ cycloalkyl, C₃-C₈ cycloalkenyl, —R^{a}cycloalkyl, —R^{a}OR⁵, —R^{a}N(R⁸)R⁹, -Ay, -Het, —R^{a}Ay, —R^{a}Het, —C(O)R⁵ or —S(O)_{q}R⁵;
each of R⁸ and R⁹ independently are selected from H or C₁-C₈ alkyl;
each q independently is 0, 1, or 2;
each Ay independently represents a C₃-C₁₀ aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; and
each Het independently represents a C₃-C₇ heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; or pharmaceutically acceptable derivatives thereof.

2. The compound of claim 1 wherein t is 1.

3. The compound of claim 1 wherein m is 0.

4. The compound of claim 1 wherein m is 1 or 2.

5. The compound of claim 4 wherein m is 1.

6. The compound of claim 5 wherein R⁴ is halogen, C₁-C₈haloalkyl, C₁-C₈alkyl, OR¹⁰NR⁶R⁷, CO₂R¹⁰, CONR⁶R⁷, or cyano.

7. The compound of claim 1 wherein R³ is halogen, —C(O)R¹¹, —C(O)N(R¹¹)R¹², —S(O)₂N(R¹¹)R¹², —N(R¹¹)C(O)N(R¹¹)R¹² or cyano.

8. The compound of claim 1 wherein R³ is —C(O)N(R¹¹)R¹² or —S(O)₂N(R¹¹)R¹².

9. The compound of claim 1 wherein R³ is halogen.

10. The compound of claim 1 wherein t is 1 or 2; R is H or C₁-C₈ alkyl; R² is H, C₁-C₈ alkyl, C₃-C₈ cycloalkyl, or —R^{a}cycloalkyl; R³ is halogen, —C(O)R¹¹, —C(O)N(R¹¹)R¹², —S(O)₂N(R¹¹)R¹² or cyano; n is 0; m is 0; p is 0 and X is -Het or -HetN(R¹⁰)₂, R¹⁰ is H or C₁-C₈ alkyl and -Het is unsubstituted or substituted with C₁-C₈ alkyl or C₃-C₈ cycloalkyl.

11. The compound of claim 1 wherein t is 1 or 2; R is H or C₁-C₈ alkyl; R² is H, C₁-C₈ alkyl, C₃-C₈ cycloalkyl, or —R^{a}cycloalkyl; R³ is halogen, —C(O)R¹¹, —C(O)N(R¹¹)R¹², —S(O)₂N(R¹¹)R¹² or cyano; n is 0; m is 0; p is 1; Y is —N(R¹⁰)—, —O—, —C(O)N(R¹⁰)—, or —N(R¹⁰)C(O)—; X is -Het or -HetN(R¹⁰)₂, and R¹⁰ is H or C₁-C₈ alkyl and Het is unsubstituted or substituted with C₁-C₈ alkyl or C₃-C₈ cycloalkyl.

12. The compound of claim 1 wherein t is 1 or 2; R is H or C₁-C₈ alkyl; R² is H, C₁-C₈ alkyl, C₃-C₈ cycloalkyl, or —R^{a}cycloalkyl; R³ is halogen, —C(O)R¹¹, —C(O)N(R¹¹)R¹², —S(O)₂N(R¹¹)R¹² or cyano; n is 0; m is 0; p is 1; Y is —N(R¹⁰)— or —O— and X is -Het, unsubstituted or substituted with C₁-C₈ alkyl or C₃-C₈ cycloalkyl.

13. The compound of claim 1 wherein the substituent —Yₚ—X is located on the depicted benzimidazole ring as in formula (I-A): 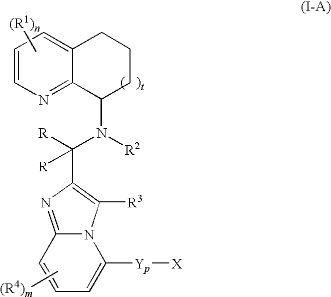
or pharmaceutically acceptable derivatives thereof.

14. The compound of claim 1 wherein Het is piperidine, piperazine, azetidine, pyrrolidine, imidazole, or pyridine.

15. The compound of claim 1 wherein p is 0 and X is -Het or -HetN(R¹⁰)₂ where R¹⁰ is H or C₁-C₈ alkyl.

16. The compound of claim 15 wherein Het is unsubstituted or substituted with one or more C₁-C₈ alkyl or C₃-C₈ cycloalkyl.

17. A compound selected from the group consisting of
5-(4-Methyl-1-piperazinyl)-2-({methyl[(8S)-5,6,7,8-tetrahydro-8-quinolinyl]amino}methyl)imidazo[1,2-a]pyridine-3-carbonitrile;
5-(4-Methyl-1-piperazinyl)-2-({methyl[(8S)-5,6,7,8-tetrahydro-8-quinolinyl]amino}methyl)imidazo[1,2-a]pyridine-3-carboxamide;
2-({Ethyl[(8S)-5,6,7,8-tetrahydro-8-quinolinyl]amino}methyl)-5-(4-methyl-1-piperazinyl)imidazo[1,2-a]pyridine-3-carbonitrile;
2-({Ethyl[(8S)-5,6,7,8-tetrahydro-8-quinolinyl]amino}methyl)-5-(4-methyl-1-piperazinyl)imidazo[1,2-a]pyridine-3-carboxamide;
5-(4-Methyl-1-piperazinyl)-2-({propyl[(8S)-5,6,7,8-tetrahydro-8-quinolinyl]amino}methyl)imidazo[1,2-a]pyridine-3-carbonitrile;
5-(4-Methyl-1-piperazinyl)-2-({propyl[(8S)-5,6,7,8-tetrahydro-8-quinolinyl]amino}methyl)imidazo[1,2-a]pyridine-3-carboxamide;
2-({(1-Methylethyl)[(8S)-5,6,7,8-tetrahydro-8-quinolinyl]amino}methyl)-5-(4-methyl-1-piperazinyl)imidazo[1,2-a]pyridine-3-carbonitrile;
2-({(1-Methylethyl)[(8S)-5,6,7,8-tetrahydro-8-quinolinyl]amino}methyl)-5-(4-methyl-1-piperazinyl)imidazo[1,2-a]pyridine-3-carboxamide;
2-({(Cyclopropylmethyl)[(8S)-5,6,7,8-tetrahydro-8-quinolinyl]amino}methyl)-5-(4-methyl-1-piperazinyl)imidazo[1,2-a]pyridine-3-carbonitrile;
5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carbonitrile;
5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridine-3-carboxamide;
5-[(3R)-3-(Dimethylamino)-1-pyrrolidinyl]-2-({(phenylmethyl)[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({methyl[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({methyl[(8S)-5,6,7,8-tetrahydro-8-quinolinyl]amino}methyl)imidazo[1,2-a]pyridine-3-carboxamide;
5-[4-(dimethylamino)-1-piperidinyl]-2-({ethyl[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({ethyl[(8S)-5,6,7,8-tetrahydro-8-quinolinyl]amino}methyl)imidazo[1,2-a]pyridine-3-carboxamide;
5-[4-(dimethylamino)-1-piperidinyl]-2-({propyl[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({propyl[(8S)-5,6,7,8-tetrahydro-8-quinolinyl]amino}methyl)imidazo[1,2-a]pyridine-3-carboxamide;
5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carbonitrile;
5-[4-(dimethylamino)-1-piperidinyl]-2-({(1-methylethyl)[(8S)-5,6,7,8-tetrahydro-8-quinolinyl]amino}methyl)imidazo[1,2-a]pyridine-3-carboxamide;
(8S)—N-{[3-Chloro-5-(4-methyl-1-piperazinyl)imidazo[1,2-a]pyridin-2-yl]methyl}-N-methyl-5,6,7,8-tetrahydro-8-quinolinamine;
(8S)—N-{[3-Chloro-5-(4-ethyl-1-piperazinyl)imidazo[1,2-a]pyridin-2-yl]methyl}-N-methyl-5,6,7,8-tetrahydro-8-quinolinamine;
(8S)—N-({3-Chloro-5-[(3R)-3-(dimethylamino)-1-pyrrolidinyl]imidazo[1,2-a]pyridin-2-yl}methyl)-N-methyl-5,6,7,8-tetrahydro-8-quinolinamine;
(8S)—N-({3-Chloro-5-[4-(dimethylamino)-1-piperidinyl]imidazo[1,2-a]pyridin-2-yl}methyl)-N-methyl-5,6,7,8-tetrahydro-8-quinolinamine;
(8S)—N-{[3-Chloro-5-(4-methylhexahydro-1H-1,4-diazepin-1-yl)imidazo[1,2-a]pyridin-2-yl]methyl}-N-methyl-5,6,7,8-tetrahydro-8-quinolinamine;
N-[3-Chloro-2-({methyl[(8S)-5,6,7,8-tetrahydro-8-quinolinyl]amino}methyl)imidazo[1,2-a]pyridin-5-yl]-N,N′,N′-trimethyl-1,2-ethanediamine;
(8S)—N-({3-Chloro-5-[(3S)-3-methyl-1-piperazinyl]imidazo[1,2-a]pyridin-2-yl}methyl)-N-methyl-5,6,7,8-tetrahydro-8-quinolinamine;
(8S)—N-({3-Chloro-5-[(3S)-3,4-dimethyl-1-piperazinyl]imidazo[1,2-a]pyridin-2-yl}methyl)-N-methyl-5,6,7,8-tetrahydro-8-quinolinamine;
(8S)—N-({3-Chloro-5-[4-(1-methylethyl)-1-piperazinyl]imidazo[1,2-a]pyridin-2-yl}methyl)-N-methyl-5,6,7,8-tetrahydro-8-quinolinamine;
(8S)—N-({3-Chloro-5-[(8aS)-hexahydropyrrolo[1,2-a]pyrazin-2(1H)-yl]imidazo[1,2-a]pyridin-2-yl}methy 1)-N-methyl-5,6, 7,8-tetrahydro-8-quinolinamine;
1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanone;
(8S)—N-{[3-Bromo-5-(4-methyl-1-piperazinyl)imidazo[1,2-a]pyridin-2-yl]methyl}-N-methyl-5,6,7,8-tetrahydro-8-quinolinamine;
(8S)—N-{[8-Fluoro-5-(4-methyl-1-piperazinyl)imidazo[1,2-a]pyridin-2-yl]methyl}-N-propyl-5,6,7,8-tetrahydro-8-quinolinamine;
(8S)—N-({5-[4-(dimethylamino)-1-piperidinyl]imidazo[1,2-a]pyridin-2-yl}methyl)-N-propyl-5,6,7,8-tetrahydro-8-quinolinamine; and pharmaceutically acceptable derivatives thereof.

18. (canceled)

19. A pharmaceutical composition comprising a compound according to claim 1 and a pharmaceutically acceptable carrier.

20. A pharmaceutical composition according to claim 19 in the form of a tablet or capsule.

21. A pharmaceutical composition according to claim 19 in the form of a liquid or suspension.

22-39. (canceled)
